(19) United States
(12) Patent Application Publication
(10) Pub. No.: US 2020/0224220 A1
(43) Pub. Date: Jul. 16, 2020

(54) ENCAPSULATED POLYNUCLEOTIDES AND METHODS OF USE

(71) Applicant: ONCORUS, INC., Cambridge, MA (US)

(72) Inventors: Mitchell H. FINER, Cambridge, MA (US); Edward M. KENNEDY, Cambridge, MA (US); Lorena LERNER, Cambridge, MA (US)

(21) Appl. No.: 16/630,990

(22) PCT Filed: Jul. 13, 2018
(86) PCT No.: PCT/US2018/042136

§ 371 Date: Jan. 14, 2020

Related U.S. Application Data
(60) Provisional application No. 62/532,886, filed on Jul. 14, 2017.
(60) Provisional application No. 62/648,651, filed on Mar. 27, 2018.

(51) Int. Cl. C12N 15/88; C12N 15/11; C12N 15/63; C12N 7/00

(52) U.S. Cl. CPC C12N 15/88; C12N 15/111; C12N 15/63; C12N 2770/32043; C12N 2770/32021; C12N 2770/32023; C12N 2770/32032; C12N 7/00

(57) ABSTRACT

The present disclosure relates to polynucleotides comprising a nucleic acid sequence encoding a replication competent viral genome, wherein the polynucleotide is capable of producing a replication competent virus when introduced into a cell by a non-viral delivery vehicle. The present disclosure further relates to the encapsulation of the polynucleotides and the use of the polynucleotides and/or particles for the treatment and prevention of cancer.

### CROSS-REFERENCE TO RELATED APPLICATIONS

This application claims priority to U.S. Provisional Application No. 62/532,886, filed Jul. 14, 2017 and 62/648,651, filed Mar. 27, 2018, the disclosures of which are each incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ONCR_005_03WO_ST25.txt. The text file is 23 KB, created on Jul. 13, 2018, and is being submitted electronically via EFS-Web.

### FIELD

The present disclosure generally relates to the fields of immunology, inflammation, and cancer therapeutics. More specifically, the present disclosure relates to particle-encapsulated, polynucleotides encoding replication-competent viral genomes. The disclosure further relates to the treatment and prevention of proliferative disorders such as cancer.

### BACKGROUND

Oncolytic viruses are replication-competent viruses with lytic life-cycle able to infect and lyse tumor cells. Direct tumor cell lysis results not only in cell death, but also the generation of an adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

However, clinical use of replication-competent viruses poses several challenges. In general, viral exposure activates innate immune pathways, resulting in a broad, non-specific inflammatory response. If the patient has not been previously exposed to the virus, this initial innate immune response can lead to the development of an adaptive anti-viral response and the production of neutralizing antibodies. If a patient has been previously exposed to the virus, existing neutralizing anti-viral antibodies can prevent the desired lytic effects. In both instances, the presence of neutralizing antibodies not only prevents viral lysis of target cells, but also renders re-administration of the viral therapeutic ineffective. These factors limit the use of viral therapeutics in the treatment of metastatic cancers, as the efficacy of repeated systemic administration required for treatment of such cancers is hampered by naturally-occurring anti-viral responses. Even in the absence of such obstacles, subsequent viral replication in non-diseased cells can result in substantial off-disease collateral damage to surrounding cells and tissues.

There remains a long-felt and unmet need in the art for compositions and methods related to therapeutic use of replication-competent virus. The present disclosure provides such compositions and methods, and more.

### SUMMARY

The present disclosure provides DNA polynucleotides encoding a self-replicating polynucleotides and related compositions and methods. In some embodiments, the polynucleotide comprises a nucleic acid sequence encoding a replication-competent viral genome, wherein the polynucleotide is capable of producing a replication-competent virus when introduced into a cell by a non-viral delivery vehicle.

In one aspect, the disclosure provides a lipid nanoparticle (LNP) comprising a recombinant DNA molecule comprising a polynucleotide sequence encoding a replication-competent viral genome, wherein the polynucleotide sequence is operably linked to a promoter sequence capable of binding a mammalian RNA polymerase II (Pol II) and is flanked by a 3′ ribozyme-encoding sequence and a 5′ ribozyme-encoding sequence, wherein the polynucleotide encoding the replication-competent viral genome is non-viral in origin.

In an embodiment, the replication-competent viral genome is a single-stranded RNA (ssRNA) virus.

In an embodiment, the replication-competent viral genome is a single-stranded RNA (ssRNA) virus is a positive sense ((+)-sense) or a negative-sense ((−)-sense) ssRNA virus.

In an embodiment, wherein the replication-competent viral genome is a (+)-sense ssRNA virus and the (+)-sense ssRNA virus is a Picornavirus.

In an embodiment, the Picornavirus is a Seneca Valley Virus (SVV) or a Coxsackievirus.

In an embodiment, contacting the LNP with a cell results in production of viral particles by the cell, and wherein the viral particles are infectious and lytic.

In an embodiment, the recombinant DNA molecule further comprises a polynucleotide sequence encoding an exogenous payload protein.

In an embodiment, the exogenous payload protein is a fluorescent protein, an enzymatic protein, a cytokine, a chemokine, or an antigen-binding molecule capable of binding to a cell surface receptor.

In an embodiment, the cytokine is selected from Flt3 ligand and IL-18.

In an embodiment, the chemokine is selected from CXCL10 and CCL4.

In an embodiment, the antigen-binding molecule is capable of binding to and inhibiting an immune checkpoint receptor.

In an embodiment, the immune checkpoint receptor is PD1.

In an embodiment, a micro RNA (miRNA) target sequence (miR-TS) cassette is inserted into the nucleic acid sequence encoding the replication-competent viral genome, wherein the miR-TS cassette comprises one or more miRNA target sequences, and wherein expression of one or more of the corresponding miRNAs in a cell inhibits replication of the replication-competent viral genome in the cell.

In an embodiment, the one or more miRNAs are selected from miR-124, miR-1, miR-143, miR-128, miR-219, miR-219a, miR-122, miR-204, miR-217, miR-137, and miR-126.

In an embodiment, the miR-TS cassette comprises one or more copies of a miR-124 target sequence, one or more copies of a miR-1 target sequence, and one or more copies of a miR-143 target sequence.

In an embodiment, the miR-TS cassette comprises one or more copies of a miR-128 target sequence, one or more copies of a miR-219a target sequence, and one or more copies of a miR-122 target sequence.

In an embodiment, the miR-TS cassette comprises one or more copies of a miR-128 target sequence, one or more copies of a miR-204 target sequence, and one or more copies of a miR-219 target sequence.

In an embodiment, the miR-TS cassette comprises one or more copies of a miR-217 target sequence, one or more copies of a miR-137 target sequence, and one or more copies of a miR-126 target sequence.

In an embodiment, the recombinant DNA molecule is a plasmid comprising the polynucleotide sequence encoding a replication-competent viral genome.

In an embodiment, the LNP comprises a cationic lipid, a cholesterol, and a neutral lipid.

In an embodiment, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

In an embodiment, the LNP comprises a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

In an embodiment, the hyaluronan is conjugated to the surface of the LNP.

In an aspect, the disclosure provides a therapeutic composition comprising a plurality of lipid nanoparticles, wherein the plurality of LNPs have an average size of about 150 nm to about 500 nm.

In an embodiment, the plurality of LNPs have an average size of about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

In an embodiment, the plurality of LNPs have an average zeta-potential of less than about −20 mV, less than about −30 mV, less than about 35 mV, or less than about −40 mV.

In an embodiment, the plurality of LNPs have an average zeta-potential of between about −50 mV to about −20 mV, about −40 mV to about −20 mV, or about −30 mV to about −20 mV.

In an embodiment, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In an embodiment, administering the therapeutic composition to a subject delivers the recombinant DNA polynucleotide to a target cell of the subject, and the recombinant DNA polynucleotide produces an infectious virus capable of lysing the target cell of the subject.

In an embodiment, the composition is delivered intravenously or intratumorally.

In an embodiment, the target cell is a cancerous cell.

In an aspect, the disclosure provides a method of inhibiting the growth of a cancerous tumor in a subject in need thereof comprising administering a therapeutic composition to the subject in need thereof, wherein administration of the composition inhibits the growth of the tumor.

In an embodiment, the administration is intratumoral or intravenous.

In an embodiment, the cancer is a lung cancer or a liver cancer.

In an aspect, the disclosure provides a recombinant DNA molecule comprising a polynucleotide sequence encoding a replication-competent viral genome, wherein the polynucleotide sequence is operably linked to promoter sequence capable of binding a mammalian RNA polymerase II (Pol II) and is flanked by a 3′ ribozyme-encoding sequence and a 5′ ribozyme-encoding sequence, wherein the polynucleotide encoding the replication-competent viral genome is non-viral in origin.

In an embodiment, the encoded virus is a single-stranded RNA (ssRNA) virus

In an embodiment, the ssRNA virus is a positive sense ((+)-sense) or a negative-sense ((−)-sense) ssRNA virus.

In an embodiment, the (+)-sense ssRNA virus is a Picornavirus.

In an embodiment, the Picornavirus is a Seneca Valley Virus (SVV) or a Coxsackievirus.

In an embodiment, the recombinant DNA molecule is capable of producing an infectious, lytic virus when introduced into a cell by a non-viral delivery vehicle.

In an embodiment, the recombinant DNA molecule further comprises a polynucleotide sequence encoding an exogenous payload protein.

In an embodiment, the exogenous payload protein is a fluorescent protein, an enzymatic protein, a cytokine, a chemokine, a ligand for a cell-surface receptor, or an antigen-binding molecule capable of binding to a cell surface receptor.

In an embodiment, the cytokine is IL-18.

In an embodiment, the ligand for a cell-surface receptor is Flt3 ligand

In an embodiment, the chemokine is selected from CXCL10 and CCL4.

In an embodiment, the antigen-binding molecule is capable of binding to and inhibiting an immune checkpoint receptor.

In an embodiment, the immune checkpoint receptor is PD1.

In an embodiment, a micro RNA (miRNA) target sequence (miR-TS) cassette is inserted into the nucleic acid sequence encoding the replication-competent viral genome, wherein the miR-TS cassette comprises one or more miRNA target sequences, and wherein expression of one or more of the corresponding miRNAs in a cell inhibits replication of the encoded virus in the cell.

In an embodiment, the one or more miRNAs are selected from miR-124, miR-1, miR-143, miR-128, miR-219, miR-219a, miR-122, miR-204, miR-217, miR-137, and miR-126.

In an embodiment, the miR-TS cassette comprises one or more copies of a miR-124 target sequence, one or more copies of a miR-1 target sequence, and one or more copies of a miR-143 target sequence.

In an embodiment, the miR-TS cassette comprises one or more copies of a miR-128 target sequence, one or more copies of a miR-219a target sequence, and one or more copies of a miR-122 target sequence.

In an embodiment, the miR-TS cassette comprises one or more copies of a miR-128 target sequence, one or more copies of a miR-204 target sequence, and one or more copies of a miR-219 target sequence.

In an embodiment, the miR-TS cassette comprises one or more copies of a miR-217 target sequence, one or more copies of a miR-137 target sequence, and one or more copies of a miR-126 target sequence.

In an embodiment, the recombinant DNA molecule is a plasmid comprising the polynucleotide sequence encoding a replication-competent viral genome.

In an aspect, the disclosure provides a recombinant DNA molecule comprising a polynucleotide sequence encoding a replication-competent viral genome, wherein the polynucleotide sequence encoding the replication-competent virus is non-viral in origin, and wherein the recombinant DNA molecule is capable of producing a replication-competent virus when introduced into a cell by a non-viral delivery vehicle.

In an embodiment, the replication-competent viral genome is a genome of a DNA virus or a genome of an RNA virus.

In an embodiment, the DNA genome or RNA genome is a double-stranded or a single-stranded virus.

In an embodiment, the single stranded genome is a positive sense ((+)-sense) or negative sense ((−)-sense) genome.

In an embodiment, the cell is a mammalian cell.

In an embodiment, the cell is a mammalian cell present in a mammalian subject.

In an embodiment, the replication-competent virus is selected from the group consisting of an adenovirus, a coxsackievirus, an equine herpes virus, a herpes simplex virus, an influenza virus, a lassa virus, a maraba virus, a measles virus, a murine leukemia virus, a myxoma virus, a newcastle disease virus, a orthomyxovirus, a parvovirus, a polio virus (including a chimeric polio virus such as PVS-RIPO), a reovirus, a seneca valley virus (e.g., Seneca A), a sindbis virus, a vaccinia virus, and a vesicular stomatitis virus.

In an embodiment, the recombinant DNA polynucleotide further comprises one or more micro RNA (miRNA) target sequence (miR-TS) cassettes inserted into the polynucleotide encoding the replication-competent viral genome, wherein the miR-TS cassette comprises one or more miRNA target sequences, and wherein expression of one or more of the corresponding miRNAs in a cell inhibits replication of the encoded virus in the cell.

In an embodiment, the one or more miR-TS cassettes is incorporated into the 5′ untranslated region (UTR) or 3′ UTR of one or more essential viral genes.

In an embodiment, the one or more essential viral genes is selected from the group consisting of UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL50, UL52, UL53, UL54, US1, US3, US4, US5, US6, US7, US8, US12, ICP0, ICP4, ICP22, ICP27, ICP47, PB, F, B5R, SERO-1, Cap, Rev, VP1-4, nucleoprotein (N), phosphoprotein (P), matrix protein (M), glycoprotein (G), polymerase (L), E1, E2, E3, E3, VP1, VP2, VP3, VP4, 2A, 2B, 2C, 3A, 3B, 3C, and 3D.

In an embodiment, the one or more miR-TS cassettes is incorporated into the 5′ untranslated region (UTR) or 3′ UTR of one or more non-essential genes.

In an embodiment, the polynucleotide is inserted into a nucleic acid vector selected from a replicon, a plasmid, a cosmid, a phagemid, a transposon, a bacterial artificial chromosome, a yeast artificial chromosome, or an end-closed linear duplexed oncolytic virus (Ov) DNA molecule.

In an embodiment, the polynucleotide is a DNA polynucleotide and further comprises a first AAV-derived inverted terminal repeat (ITR) on the 5′ end of the nucleic acid sequence encoding the replication-competent viral genome and a second AAV-derived ITR on the 3′ end of the nucleic acid sequence encoding the replication-competent viral genome.

In an embodiment, the polynucleotide is a DNA polynucleotide and further comprises a first ribozyme encoding sequence immediately 3′ to the nucleic acid sequence encoding the replication-competent viral genome and a second ribozyme encoding sequence immediately 5′ to the nucleic acid sequence encoding the replication-competent viral genome.

In an embodiment, the first and second ribozyme encoding sequences encode a Hammerhead ribozyme or a hepatitis delta virus ribozyme.

In an embodiment, the promoter sequence is capable of binding a eukaryotic RNA polymerase.

In an embodiment, the promoter sequence is capable of binding a mammalian RNA polymerase.

In an embodiment, the polynucleotide is a DNA polynucleotide and the mammalian polymerase drives the transcription of an infectious, replication-competent RNA virus.

In an embodiment, the polynucleotide is a DNA polynucleotide and the mammalian polymerase drives the transcription of an infectious, replication-competent DNA virus.

In an embodiment, the promoter sequence selectively drives transcription of the polynucleotide in a cancer cell.

In an embodiment, the promoter sequence is derived a gene selected from the group consisting of hTERT, HE4, CEA, OC, ARF, CgA, GRP78, CXCR4, HMGB2, INSM1, Mesothelin, OPN, RAD51, TETP, H19, uPAR, ERBB2, MUC1, Frz1, or IGF2-P4.

In an embodiment, the recombinant DNA polynucleotide further comprises a nucleic acid sequence encoding a payload molecule selected from the group consisting of a cytotoxic polypeptide, a cytokine, a chemokine, an antigen binding molecule, a ligand for a cell surface receptor, a soluble receptor, an enzyme, a scorpion polypeptide, a snake polypeptide, a spider polypeptide, a bee polypeptide, a frog polypeptide, and a therapeutic nucleic acid.

In an embodiment, one or more miR-TS cassettes is incorporated into the 5′ untranslated region (UTR) or the 3′ UTR sequence of the nucleic acid sequence encoding the payload molecule.

In an embodiment, the cytotoxic polypeptide is selected from p53, diphtheria toxin (DT), Pseudomonas Exotoxin A (PEA), Type I ribosome inactivating proteins (RIPs), Type II RIPs, or Shiga-like toxin 1 (Slt1).

In an embodiment, the enzyme is selected from a metalloproteinase, a collagenase, an elastase, a hyaluronidase, a caspase, a gelatinase, or an enzyme that is part of a gene directed enzyme prodrug therapy (GDEPT) system selected from herpes simplex virus thymidine kinase, cytosine deaminase, nitroreductase, carboxypeptidase G2, purine nucleoside phosphorylase, or cytochrome P450.

In an embodiment, the gelatinase is fibroblast activation protein (FAP).

In an embodiment, the metalloproteinase is a matrix metalloproteinase (e.g., MMP9) or ADAM17.

In an embodiment, the cytokine is selected from the group consisting of osteopontin, IL-13, TGFβ, IL-35, IL-18, IL-15, IL-2, IL-12, IFNα, IFNβ, IFNγ.

In an embodiment, the chemokine is selected from CXCL10, CCL4, CCL5, CXCL9, and CCL21.

In an embodiment, the ligand for a cell-surface receptor is an NKG2D ligand, a neuropilin ligand, Flt3 ligand, a CD47 ligand.

In an embodiment, the antigen-binding molecule binds to a cell-surface antigen selected from the group consisting of PD-1, PDL-1, CTLA4, CCR4, OX40, CD200R, CD47, CSF1R, EphA2, CD19, EpCAM, CEA, PSMA, CD33, EGFR, CCR4, CD200, CD40, CD47, HER2, DLL3, 4-1BB, 17-1A, GD2 and any one or more of the tumor antigens listed in Table 7.

In an embodiment, the scorpion polypeptide is selected from the group consisting of chlorotoxin, BmKn-2, neopladine 1, neopladine 2, and mauriporin.

In an embodiment, the snake polypeptide is selected from the group consisting of contortrostatin, apoxin-I, bothropstoxin-I, BJcuL, OHAP-1, rhodostomin, drCT-I, CTX-III, B1L, and ACTX-6.

In an embodiment, the spider polypeptide is selected from the group consisting of latarcin and hyaluronidase.

In an embodiment, the bee polypeptide is selected from the group consisting of melittin and apamin.

In an embodiment, the frog polypeptide is selected from the group consisting of PsT-1, PdT-1, and PdT-2.

In an embodiment, the payload protein acts on an immune cell.

In an embodiment, the immune cell is selected from a group consisting of a T cell, a B cell, a natural killer (NK) cell, an NKT cell, a macrophage, and/or a dendritic cell.

In an embodiment, the payload polypeptide is a bipartite polypeptide comprising a first domain capable of binding a human cell surface antigen and a second domain capable of binding a human tumor cell antigen.

In an embodiment, one or both domains of the bipartite polypeptide are antigen-binding molecules selected from the group consisting of an antibody, a single chain variable fragment (scFv), an F(ab), an immunoglobulin heavy chain variable domain, a diabody, a flexibody, a DOCK-AND-LOCK™ antibody, and a monoclonal anti-idiotypic antibody (mAb2).

In an embodiment, the bipartite polypeptide is a dual-variable domain antibody (DVD-Ig™), a bi-specific T cell engager (BiTE™), a DuoBody®, a dual affinity retargeting (DART) polypeptide, or a Tandab®.

In an embodiment, the antibody is an IgG antibody with an engineered Fc domain.

In an embodiment, the therapeutic nucleic acid is an antagomir, a short-hair pin RNA (snRNA), a ribozyme, or an aptamer.

In an embodiment, the polynucleotide does not replicate in or minimally replicates in a cell expressing a miRNA that binds to the miRNA target sequences comprised in the miR-TS cassette.

In an embodiment, the miRNA is selected from Table 3.

In an embodiment, the one or more miRNAs are selected from miR-124, miR-1, miR-143, miR-128, miR-219, miR-219a, miR-122, miR-204, miR-217, miR-137, and miR-126.

In an embodiment, the miR-TS cassette comprises one or more copies of a miR-124 target sequence, one or more copies of a miR-1 target sequence, and one or more copies of a miR-143 target sequence.

In an embodiment,the miR-TS cassette comprises one or more copies of a miR-128 target sequence, one or more copies of a miR-219a target sequence, and one or more copies of a miR-122 target sequence.

In an embodiment, the miR-TS cassette comprises one or more copies of a miR-128 target sequence, one or more copies of a miR-204 target sequence, and one or more copies of a miR-219 target sequence.

In an embodiment, the miR-TS cassette comprises one or more copies of a miR-217 target sequence, one or more copies of a miR-137 target sequence, and one or more copies of a miR-126 target sequence.

In an embodiment, the recombinant DNA molecule is a plasmid comprising the self-replicating polynucleotide.

In an aspect, the disclosure provides a recombinant DNA molecule comprising a first single-stranded DNA (ssDNA) molecule comprising a sense sequence of a viral genome; and a second ssDNA molecule comprising an anti-sense sequence of the viral genome, wherein each of the first and second ssDNA molecules comprise a 3′ inverted terminal repeat and a 5′ inverted terminal repeat and wherein the 3′ end of the sense ssDNA molecule is covalently linked to the 5′ end of the anti-sense ssDNA molecule, and the 5′ end of the sense ssDNA molecule is covalently linked to the 3′ end of the anti-sense ssDNA molecule to form an end-closed linear duplexed oncolytic virus (Ov) DNA molecule.

In an embodiment, the encoded virus is a negative-sense or a positive-sense single stranded (ss) RNA virus.

In an embodiment, the positive-sense ssRNA virus is a polio virus (PV).

In an embodiment, the negative-sense ssRNA virus is a vesicular stomatitis virus (VSV) genome.

In an embodiment, each of the first and second ssDNA molecules further comprises a ribozyme-encoding sequence immediately 5′ to the viral genome sequence and a ribozyme-encoding sequence immediately 3′ to the viral genome sequence.

In an embodiment, the viral genome comprises one or more micro-RNA (miRNA) target sequences inserted into one or more essential viral genes.

In an embodiment, the one or more miRNA target sequences are inserted into the 3′ untranslated region (UTR) and/or the 5′ UTR of the one or more essential viral genes.

In an embodiment, the one or more miRNA target sequences are inserted into at least 2, at least 3, at least 4, or more essential viral genes.

In an embodiment, at least 2, at least 3, or at least 4 miRNA target sequences are inserted into one or more essential viral genes.

In an embodiment, the at least 2, at least 3, or at least 4 miRNA target sequences comprise target sequences for one miRNA.

In an embodiment, the at least 2, at least 3, or at least 4 miRNA target sequences comprise target sequences for at least 2, at least 3, or at least 4 different miRNAs.

In an embodiment, the viral genome is a VSV genome, and wherein the one or more miRNA target sequences are inserted into one or more of the genes encoding nucleoprotein (N), phosphoprotein (P), matrix protein (M), glycoprotein (G), and/or polymerase (L) proteins.

In an embodiment, the viral genome is a PV genome, and wherein the one or more miRNA target sequences are inserted in one or more of the genes encoding the VP1, VP2, VP3, VP4, 2A, 2B, 2C, 3A, 3B (VPg), 3C, or 3D proteins.

In an embodiment, the 3′ and 5′ ITRs are derived from AAV.

In an embodiment, the AAV is AAV2.

In an aspect, the disclosure provides a composition comprising an effective amount of the recombinant DNA molecule and a carrier suitable for administration to a mammalian subject.

In an aspect, the disclosure provides a particle comprising any recombinant DNA molecule of the disclosure.

In an embodiment, the particle is biodegradable.

In an embodiment, the particle is selected from the group consisting of a nanoparticle, an exosome, a liposome, and a lipoplex.

In an embodiment, the exosome is a modified exosome derived from an intact exosome or an empty exosome.

In an embodiment, the nanoparticle is a lipid nanoparticle (LNP) comprising a cationic lipid, a cholesterol, and a neutral lipid.

In an embodiment, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

In an embodiment, the LNP further comprises a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

In an embodiment, hyaluronan is conjugated to the surface of the LNP.

In an aspect, the disclosure provides a therapeutic composition comprising a plurality of lipid nanoparticles, wherein the plurality of LNPs have an average size of about 150 nm to about 500 nm.

In an embodiment, the plurality of LNPs have an average size of about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

In an embodiment, the plurality of LNPs have an average zeta-potential of less than about −20 mV, less than about −30 mV, less than about 35 mV, or less than about −40 mV.

In an embodiment, the plurality of LNPs have an average zeta-potential of between about −50 mV to about −20 mV, about −40 mV to about −20 mV, or about −30 mV to about −20 mV.

In an embodiment, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In an embodiment, delivery of the composition to a subject delivers the encapsulated DNA expression cassette to a target cell, and wherein the encapsulated DNA expression cassette produces an infectious virus capable of lysing the target cell.

In an embodiment, the composition is delivered intravenously or intratumorally.

In an embodiment, the target cell is a cancerous cell.

In an aspect, the disclosure provides an inorganic particle comprising any polynucleotide of the disclosure.

In an embodiment, the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

In an embodiment, the average diameter of the particles is less than about 500 nm, is between about 250 nm and about 500 nm, or is about 350 nm.

In an aspect, the disclosure provides a method of killing a cancerous cell comprising exposing the cancerous cell to the particle or composition of any one of claims 122-140, or a composition thereof, under conditions sufficient for the intracellular delivery of the particle to said cancerous cell, wherein the replication-competent virus produced by the encapsulated polynucleotide results in killing of the cancerous cell.

In an embodiment, the replication-competent virus is not produced in non-cancerous cells.

In an embodiment, the method is performed in vivo, in vitro, or ex vivo.

In an aspect, the disclosure provides a method of treating a cancer in a subject comprising administering to a subject suffering from the cancer an effective amount of the particle or composition of any one of claims 122-140, or a composition thereof.

In an embodiment, the particle or composition thereof is administered intravenously, intranasally, as an inhalant, or is injected directly into a tumor.

In an embodiment, the particle or composition thereof is administered to the subject repeatedly.

In an embodiment, the subject is a mouse, a rat, a rabbit, a cat, a dog, a horse, a non-human primate, or a human.

In an embodiment, the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), and marginal zone lymphoma (MZL).

In an embodiment, the lung cancer is small cell lung cancer or non-small cell lung cancer.

In an embodiment, the liver cancer is hepatocellular carcinoma (HCC).

In an aspect, the disclosure provides a method of producing a recombinant DNA molecule of any of the preceding claims comprising inserting the recombinant DNA molecule into a first viral expression vector, wherein the recombinant DNA molecule comprises a 5′ adeno-associated virus (AAV)-derived inverted terminal repeat (ITR) and a 3′ AAV-derived ITR end of the polynucleotide; inserting polynucleotides encoding AAV proteins required for ITR-mediated replication into a second viral expression vector; and intracellularly delivering the first and the second viral expression vectors to a cell, wherein the recombinant DNA molecule is stably integrated into the genome, wherein the cell produces the ITR-flanked polynucleotides in amounts greater than would be produced in the absence of ITRs.

In an embodiment, the viral expression vector is a herpes virus or a baculovirus.

### BRIEF DESCRIPTION OF THE DRAWINGS

FIG. 1 shows examples of the diverse variety of DNA or RNA viruses from which polynucleotide genomes may be derived.

FIG. 2 shows an example of a lipid based nanoparticle coated with the glycosaminoglycan (CAG) hyaluronan (HA) into which self-replicating polynucleotides are encapsulated (http://www.quietx.com).

FIG. 3 shows an example of treatment of cancer with a self-replicating polynucleotide encapsulated in a tumor targeted nanoparticle.

FIG. 4A-FIG. 4B show examples of replicating HSV vectors for propagation of self-replicating viral genomes comprising 5′ and 3′ ITRs with Rep 52 and Rep 78 expressed in trans (FIG. 4A) and self-replicating viral genomes comprising 5′ and 3′ ITRs with an internal Rep cassette (FIG. 4B). gB:NT=virus entry-enhancing double mutation in gB gene; BAC=loxP-flanked choramphenicol-resistance and lacZ sequences; ΔJoint=deletion of the complete internal repeat region including one copy of the ICP4 gene; ITR=inverted terminal repeats derived from AAV; Pol IIp=Constitutive Pol II promoter; Rep cassette=cassette encoding AAV Rep 52 and Rep 78 for replication of ITR-flanked viral genome DNA; optional miRNA attenuation indicated by diagonally hashed boxes.

FIG. 5A-FIG. 5B show examples of example of non-replicating HSV vectors for propagation of self-replicating polynucleotides comprising 5′ and 3′ ITRs with Rep 52 and Rep 78 expressed in trans (FIG. 5A) and self-replicating viral genomes comprising 5′ and 3′ ITRs with an internal Rep cassette (FIG. 5B). gB:NT=virus entry-enhancing double mutation in gB gene; BAC=loxP-flanked choramphenicol-resistance and lacZ sequences; ΔJoint=deletion of the complete internal repeat region including one copy of the ICP4 gene; ITR=inverted terminal repeats derived from AAV; Pol IIp=Constitutive Pol II promoter; Rep cassette=cassette encoding AAV Rep 52 and Rep 78 for replication of ITR-flanked viral genome DNA; optional miRNA attenuation indicated by diagonally hashed boxes.

FIG. 6A-FIG. 6B show illustrations of a polynucleotide encoding a positive stranded RNA polio virus type I genome. The polynucleotide may be optionally flanked on the 5′ and 3′ ends by AAV-derived ITRs (FIG. 6A and FIG. 6B). The polynucleotide may optionally comprise one or more miRNA target sequence cassettes (miR TS cassette) for miRNA attenuation (FIG. 6B).

FIG. 7A-FIG. 7B show examples of replicating HSV vectors for the production of self-replicating polynucleotides encoding polio virus type I genomes. The polio virus genomes may optionally comprise miRNA target sites for miRNA-attenuation (indicated by diagonally hashed boxes). FIG. 7B illustrates a replicating HSV vector for the production of self-replicating polynucleotides encoding polio virus type I genomes flanked on the 5′ and 3′ ends by AAV-derived ITRs. gB:NT=virus entry-enhancing double mutation in gB gene; BAC=loxP-flanked choramphenicol-resistance and lacZ sequences; ΔUL19=deletion of the UL19 gene encoding the major capsid protein, VP5; ΔJoint=deletion of the complete internal repeat region including one copy of the ICP4 gene; Pol IIp=Constitutive RNA Pol II promoter; Rep cassette=cassette encoding AAV Rep 52 and Rep 78 for replication of ITR-flanked viral genome DNA; Polio viral genome cassette=inserted into intergenic locus of HSV genome, plus strand genome produced by transcription; optional miRNA attenuation indicated by diagonally hashed boxes.

FIG. 8A-FIG. 8C show examples of polio virus type I polynucleotide genomes for the treatment of particular cancers such as non-small cell lung cancer (FIG. 8A), hepatocellular carcinoma (FIG. 8B), and prostate cancer (FIG. 8C).

FIG. 9A-FIG. 9B show examples of self-replicating polynucleotides encoding vesicular stomatitis virus (VSV) genomes. The polynucleotide may be optionally flanked on the 5′ and 3′ ends by AAV-derived ITRs (FIG. 9B). The polynucleotide may optionally comprise one or more miRNA target sequences for miRNA attenuation, indicated by diagonally hashed boxes (FIG. 9B).

FIG. 10A-FIG. 10B show examples of replicating HSV vectors for the production of VSV genome polynucleotide genomes. The VSV genomes may optionally comprise miRNA target sites for miRNA-attenuation (FIG. 10A and FIG. 10B). FIG. 10B illustrates a replicating HSV vector for the production of VSV genomes flanked on the 5′ and 3′ ends by AAV-derived ITRs. gB:NT=virus entry-enhancing double mutation in gB gene; BAC=loxP-flanked choramphenicol-resistance and lacZ sequences; ΔJoint=deletion of the complete internal repeat region including one copy of the ICP4 gene; ΔUL19=deletion of the UL19 gene encoding the major capsid protein, VPS; VSV genome cassette=antigenomic (negative strand) VSV genome and mammalian expression cassette encoding essential VSV genes, N, P, and L with bi-directional Pol II promoter (BD Pol IIp) for transcription of negative strand VSV genome and essential VSV genes inserted into intergenic locus of HSV genome; optional miRNA attenuation indicated by diagonally hashed boxes; Rep cassette=cassette encoding AAV Rep 52 and Rep 78 for replication of ITR-flanked viral genome DNA; Pol IIp=Constitutive Pol II promoter.

FIG. 11A-FIG. 11C show examples of VSV polynucleotide genomes for the treatment of particular cancers such as hepatocellular carcinoma (FIG. 11A), prostate cancer (FIG. 11B), and non-small cell lung cancer (FIG. 11C).

FIG. 12A-FIG. 12B show examples of adenovirus polynucleotide genomes. The AAV genome may optionally comprise miRNA target sites for miRNA-attenuation, indicated by diagonally hashed boxes (FIG. 12B).

FIG. 13A-FIG. 13C show examples of AAV polynucleotide genomes for the treatment of particular cancers such as hepatocellular carcinoma (FIG. 13A), prostate cancer (FIG. 13B), and non-small cell lung cancer (FIG. 13C)

FIG. 14 shows a schematic of the CVB3 viral genome. CVB3 is a +sense, ssRNA Picornavirus with a genome size of ˜7.4 kb.

FIG. 15 shows a schematic of a Coxsackievirus A21 construct.

FIG. 16 shows a schematic of a Seneca Valley virus (SVV) construct.

FIG. 17 shows a recombinant HSV-1, bacterial artificial chromosome (BAC) vector comprising an ITR-flanked oncolytic virus (OV) DNA cassette and a Rep cassette

FIG. 18 show control of Rep expression by Rep cassette and the A/C heterodimerizer, AP21967.

FIG. 19A-FIG. 19D show monomers and dimers of the NanoV constructs produced by the system shown in FIG. 17. FIG. 19A shows structure and sizes of NanoV monomers and dimers. FIG. 19B shows gel analysis of predicted monomers and dimers after restriction enzyme digestion. FIG. 19C shows a schematic of the NanoV construct with locations of internal PCR primers. FIG. 19D shows PCR amplification of NanoV using internal primers.

FIG. 20A-FIG. 20C show production of NanoV concatamers in predicted orientations. FIG. 20A shows the location of the AflII cleavage site in the NanoV monomer. FIG. 20B shows the possible concatamer orientations and predicted sizes of AflII cleavage products. FIG. 20C shows gel analysis of AflII-digested NanoV DNA.

FIG. 21 shows expression of mCherry from NanoV DNA construct.

FIG. 22 shows a schematic of a Picornavirus construct comprising 3′ and 5′ ribozyme sequences.

FIG. 23A-FIG. 23B depict schematics of the design and culture protocol of a polynucleotide encoding a replication-competent Seneca valley virus (SVV). FIG. 23A shows a capped polyadenylated transcript comprising mammalian 5′ and 3′ UTR sequences, a hammerhead ribozyme, and a hepatitis delta ribozyme. FIG. 23B shows a schematic of the culture protocol for production of the infectious SVV.

FIG. 24 shows crystal violet staining demonstrating lysis of the monolayer from virus produced from 293T cells transfected dsDNA encoding SVV-ribozymes (WT) and SVV-mCherry-ribozymes.

FIG. 25A-FIG. 25C illustrates expression of three different exogenous payloads from the SVV transcript shown in FIG. 23. FIG. 20A shows bright field and fluorescent microscopy for mCherry. FIG. 20B shows the results of a nanoluciferase assay. FIG. 25C shows CXCL10 expression.

FIG. 26 shows miRNA attenuation of SVV-encoding plasmid constructs.

FIG. 27A-FIG. 27B show in vivo production of infectious virus and inhibition of tumor growth by SVV-encoding DNA plasmids delivered intratumorally. FIG. 27A shows inhibition of tumor growth after intratumoral administration of SVV-encoding plasmids. FIG. 27B shows isolation of live virus from pulverized tumors harvested from the experiment shown in FIG. 27A.

FIG. 28A-FIG. 28B show in vivo expression exogenous payloads by SVV-encoding DNA plasmids delivered intratumorally. FIG. 22A shows average radiance detected in tumor lysates after intratumoral injection of plasmid DNA. FIG. 22B shows CXCL10 levels detected in tumor lysates after intratumoral injection of plasmid DNA.

FIG. 29 shows delivery of SVV-encoding plasmids to tumor sites after intravenous delivery.

FIG. 30 shows inhibition of tumor growth after intravenous delivery of LNP-encapsulated SVV-encoding plasmid DNA.

FIG. 31A shows a map of an SVV-encoding plasmid. FIG. 31B shows a map of an CVA21-encoding plasmid.

FIG. 32A-FIG. 32B illustrate systems for producing +sense ssRNA viral genomes with discrete 3′ and 5′ native ends.

### DETAILED DESCRIPTION

There is a need in the art for self-replicating viral therapies that are effective in the presence of neutralizing antibodies, able to be repeatedly systemically administered, and whose replication is limited to diseased cells, thus maximizing therapeutic efficacy while minimizing collateral damage to normal, non-cancerous cells. The present disclosure overcomes these obstacles and provides for polynucleotides encoding replication-competent viral genomes that can be encapsulated in a non-immunogenic particle, such as a lipid nanoparticle, polymeric nanoparticle, or an exosome. In some embodiments, the present disclosure provides for recombinant DNA molecules encoding replication-competent viruses and methods of use for the treatment and prevention of proliferative diseases and disorders (e.g., cancer). In certain embodiments, the recombinant DNA molecule further comprises a polynucleotide sequence encoding a therapeutic molecule. The present disclosure enables the systemic delivery of a safe, efficacious recombinant polynucleotide vector suitable to treat a broad array of proliferative disorders (e.g., cancers).

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### I. Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99 or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

“Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a DNA polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject.

“Treating” as used herein refers to delivering an agent or composition to a subject to affect a physiologic outcome. In some embodiments, treatment comprises delivering a population of cells (e.g., a population of modified immune effector cells) to a subject. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and (c) curing the disease.

The term “effective amount” refers to the minimum amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Effector function” refers to functions of an immune cell related to the generation, maintenance, and/or enhancement of an immune response against a target cell or target antigen.

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a self-replicating polynucleotide or a particle-encapsulated self-replicating polynucleotide described herein that is capable of being administered or delivered to a subject or cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The term “self-replicating polynucleotides” refers to exogenous polynucleotides that are capable of replicating within a host cell in the absence of additional exogenous polynucleotides or exogenous vectors.

The term “replication-competent viral genome” refers to a viral genome encoded by the self-replicating polynucleotides described herein, which encodes all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable transferring or transporting another nucleic acid molecule.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### II. Self-Replicating Polynucleotides

In some embodiments, the present disclosure provides a recombinant nucleic acid molecule comprising a polynucleotide encoding a replication-competent viral genome that is capable producing an infectious, lytic virus when introduced into a cell by a non-viral delivery vehicle. The self-replicating polynucleotides described herein do not require additional exogenous genes or proteins to be present in the cell in order to replicate and produce infectious virus. Rather, the endogenous transcription mechanisms in the host cell mediate the initial first round of transcription or translation of the self-replicating polynucleotides to produce a replication-competent viral genome. The viral genomes encoded by the self-replicating polynucleotides are able to express the viral proteins necessary for continued replication of the viral genome and assembly into an infectious viral particle (which may comprise a capsid protein, an envelope protein, and/or a membrane protein) comprising the replication-competent viral genome. As such, the replication-competent viral genomes encoded by the self-replicating polynucleotides described herein are capable of producing a virus that is capable of infecting a host cell.

In some embodiments, the recombinant nucleic acid molecule is a recombinant DNA molecule comprising a DNA polynucleotide encoding a replication-competent viral genome. In some embodiments, the recombinant DNA molecule is a replicon, a plasmid, a cosmid, a phagemid, a transposon, a bacterial artificial chromosome, or a yeast artificial chromosome. In some embodiments, the recombinant DNA molecule is a plasmid comprising a self-replicating polynucleotide.

In some embodiments, the recombinant nucleic acid molecules described herein comprise a self-replicating polynucleotide (e.g., a polynucleotide encoding a replication-competent viral genome) that is operably linked to a transcriptional control element, such as a promoter that drives or modulates transcription of the self-replicating polynucleotide. In some embodiments, the transcriptional control element is a mammalian promoter sequence. In some embodiments, the mammalian promoter sequence is capable of binding a mammalian RNA polymerase. For example, in some embodiments, the mammalian promoter sequence is an RNA polymerase II (Pol II) promoter. In some embodiments, the mammalian promoter is a constitutive promoter, such as a CAG, a UbC, a EF1a, or a PGK promoter. In some embodiments, the transcriptional control element is a phage-derived promoter sequence, such as a T7 promoter. In such embodiments, polynucleotides under the control of a T7 promoter are transcribed in the cytosol of a cell.

In some embodiments, the promoter is an inducible promoter, such as a tetracycline-inducible promoter (e.g., TRE-Tight), a doxycline-inducible promoter, a temperature-inducible promoter (e.g., Hsp70 or Hsp90-derived promoters), a lactose-inducible promoter (e.g., a pLac promoter). In some embodiments, the promoter sequence comprises one or more transcriptional enhancer elements that modulate transcription. For example, in some embodiments, the promoter comprises one or more hypoxia responsive elements or one or more radiation responsive elements. In some embodiments, the promoter drives transcription of the self-replicating polynucleotide predominantly in cancer cells. For example, in some embodiments, the transcriptional control element is a promoter derived from a gene whose expression is increased in cancer cells, such as hTERT, HE4, CEA, OC, ARF, CgA, GRP78, CXCR4, HMGB2, INSM1, Mesothelin, OPN, RAD51, TETP, H19, uPAR, ERBB2, MUC1, Frz1, IGF2-P4, Myc, or E2F.

In some embodiments, the recombinant nucleic acid molecules described herein comprise a polynucleotide encoding a replication-competent viral genome, wherein the polynucleotide is flanked on the 5′ and 3′ ends by inverted terminal repeat (ITR) sequences. Herein, the term “inverted terminal repeat” or “ITR” refers to a polynucleotide sequence located at the 3′ and/or 5′ terminal ends of a heterologous polynucleotide sequence (e.g., a nucleic acid sequence encoding a replication-competent viral genome) and comprising palindromic sequences separated by one or more stretches of non-palindromic sequences. A “palindromic” sequence refers to a nucleic acid sequence that is identical to its complementary strand when both are read in the 5′ to 3′ direction. The polynucleotide sequences of the ITRs will form a stem-loop structure (e.g., a hair-pin loop) by way of complementary base pairing between the palindromic sequences. The ITR polynucleotide sequences can be any length, so long as the sequence is able to form a stem-loop structure. In some embodiments, the polynucleotides comprise the following structures:

5′-ITR-sense viral genome-ITR-3′; or

3′-ITR-anti-sense viral genome-ITR-5′.

In some embodiments, the ITR sequences described herein minimally comprise a palindromic sequence capable of forming a stem-loop structure, a Rep-binding site, and a terminal resolution site. In some embodiments, the ITRs described herein are derived from an adeno-associated virus (AAV). In such embodiments, the ITRs may be derived from any known serotype of AAV (e.g., AAV1, 2, 3, 4, 5, 6, 7, 8, 9, 10, or 11) (See e.g., U.S. Pat. No. 9,598,703). In some embodiments, the ITRs described herein may be derived from a parvovirus (See e.g., U.S. Pat. No. 5,585,254). Additional inverted terminal repeat sequences suitable for use in the present disclosure are described in International PCT Publication Nos. WO 2017/152149 and WO 2016/172008, and US Patent Application Publication No. US 2017-0362608.

In some embodiments, the recombinant nucleic acid molecule described herein comprise two ITR-flanked polynucleotide molecules, wherein the 5′ ITR of the first molecule is covalently linked to the 3′ ITR of the second molecule and the 3′ ITR of the first molecule is covalently linked to the 5′ ITR of the second molecule. In such embodiments, the covalently linked ITR-flanked polynucleotides form an end-closed, linear duplexed oncolytic virus nucleic acid molecule. In some embodiments, the recombinant nucleic acid molecule described herein comprise (i) a first single-stranded DNA (ssDNA) molecule comprising a polynucleotide encoding a sense sequence of a viral genome; and (ii) a second ssDNA molecule comprising a polynucleotide encoding an anti-sense sequence of the viral genome, wherein each of the first and second ssDNA molecules comprise a 3′ ITR and a 5′ ITR, wherein the 3′ end of the first ssDNA molecule is covalently linked to the 5′ end of the second ssDNA molecule, and the 5′ end of the first ssDNA molecule is covalently linked to the 3′ end of the second ssDNA molecule to form an end-closed linear duplexed oncolytic virus (Ov) DNA molecule, referred to herein as a “NanoV molecule.”.

In some embodiments, the self-replicating polynucleotide encodes a replication-competent DNA or RNA viral genome. In some embodiments, the replication-competent viral genome is a single stranded genome (e.g., an ssRNA genome or ssDNA genome). In such embodiments, the single-stranded genome may be a positive sense or negative sense genome. In some embodiments, the replication-competent viral genome is a double-stranded genome (e.g., an dsRNA genome or dsDNA genome). In some embodiments, the self-replicating polynucleotide encodes a replication-competent oncolytic virus. As used herein, the term “oncolytic virus” refers to a virus that has been modified to, or naturally, preferentially infect cancer cells. Examples of oncolytic viruses are known in the art including, but not limited to, herpes simplex virus, an adenovirus, a polio virus, a vaccinia virus, a measles virus, a vesicular stomatitis virus, an orthomyxovirus, a parvovirus, a maraba virus, or a coxsackievirus.

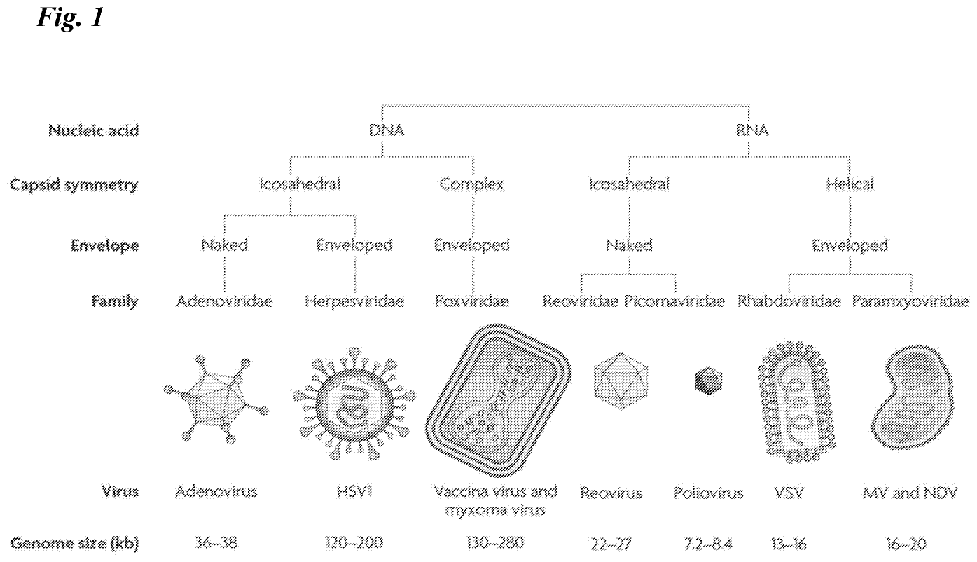

In some embodiments, the replication-competent virus produced by the polynucleotide is an any virus in the Adenoviridae family such as an Adenovirus, any virus in the family Picornaviridae family such as coxsackie virus, a polio virus, or a Seneca valley virus, any virus in the Herpesviridae family such as an equine herpes virus or herpes simplex virus type 1 (HSV-1), any virus in the Arenaviridae family such a lassa virus, any virus in the Retroviridae family such as a murine leukemia virus, any virus in the family Orthomyxoviridae such as influenza A virus, any virus in the family Paramyxoviridae such as Newcastle disease virus or measles virus, any virus in the Parvoviridaefamily, any virus in the Reoviridae family such as mammalian orthoreovirus, any virus in the Togaviridae family such as sindbis virus, any virus in the Poxviridae family such as a vaccinia virus or a myxoma virus, or any virus in the Rhabdoviridae family such as vesicular stomatitis virus (VSV) or a maraba virus, examples of which are shown in FIG. 1. In some embodiments, the replication-competent virus produced by the polynucleotide is a chimeric virus, such as a modified polio virus (e.g., PVS-RIPO).

In some embodiments, the recombinant nucleic acid molecules disclosed herein when the recombinant nucleic acid molecule is introduced into a cell are transcribed by the endogenous polymerase(s) of the cell to produce viral genomes capable of assembling into infectious viruses. The amount of infectious virus produced can be measured by methods known in the art, including but not limited to, quantifying the amount of viral RNA or viral DNA present in the target cell or population of target cells, in the supernatant of cell grown in culture, or in the tissue of a subject. In such embodiments, the total DNA or RNA can be isolated from the target cells and qPCR can be performed using primers specific for an RNA or DNA sequence present in the viral genome. In some embodiments, the number of viral particles produced from a population of cells in recombinant nucleic acids are introduced to a population of target cells (e.g., in vitro sample or a sample isolated from an in vivo tumor) can be quantified by methods known in the art. In some embodiments, formulation of the present disclosure comprise 50% Tissue culture Infective Dose (TCID₅₀) of at least about 10³-10⁹ TCID₅₀/mL, for example, at least about 10³ TCID₅₀/mL, about 10⁴ TCID₅₀/mL, about 10⁵ TCID₅₀/mL, about 10⁶ TCID₅₀/mL, about 10⁷ TCID₅₀/mL, about 10⁸ TCID₅₀/mL, or about 10⁹ TCID₅₀/mL. In some embodiments, formulation of the present disclosure significantly inhibit tumor growth in vivo.

In some embodiment, the recombinant nucleic acid molecules disclosed herein comprise a polynucleotide sequence at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to SEQ ID NOs: 1-2.

A. Single-Stranded RNA Viruses

In some embodiments, the self-replicating polynucleotides described herein encode a single-stranded RNA (ssRNA) viral genome. In some embodiments, the ssRNA virus is a positive-sense, ssRNA (+sense ssRNA) virus or a negative-sense, ssRNA (−sense ssRNA) virus.

1. Positive-Sense, Single-Stranded RNA Viruses

In some embodiments, the self-replicating polynucleotides described herein encode a positive-sense, single-stranded RNA (+sense ssRNA) viral genome. Exemplary +sense ssRNA viruses include members of the Picornaviridae family (e.g. coxsackievirus, poliovirus, and Seneca Valley virus (SVV), including SVV-A), the Coronaviridae family (e.g., Alphacoronaviruses such as HCoV-229E and HCoV-NL63, Betacoronoaviruses such as HCoV-HKU1, HCoV-OC3, and MERS-CoV), the Retroviridae family (e.g., Murine leukemia virus), and the Togaviridae family (e.g., Sindbis virus). Additional exemplary genera of and species of positive-sense, ssRNA viruses are shown below in Table 4.

TABLE 4
Positive-sense ssRNA Viruses

| Family/Subfamily | Genus | Natural Host | Species |
|---|---|---|---|
| Picornaviridae | Cardiovirus | Human | |
| | Cosavirs | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley |
| | | | Virus A |
| Caliciviridae | Sapovirus | Human | |
| | Norovirus | Human | |
| | Nebovirus | Bovine | |
| | Vesivirus | Felines/Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/ | Alphacoronavirus | | HCoV-229E |
| Coronavirinae | | | HCoV-NL63 |
| | Betacoronavirus | | HCoV-HKU1 |
| | | | HCoV-OC3 |
| | | | MERS-CoV |
| | Deltacoronavirus | | |
| | Gammacoronavirus | | |
| Coronaviridae/ | Bafinivirus | | |
| Torovirinae | Torovirus | | |
| Retroviridae | Gammaretrovirus | | Murine leukemia |
| | | | virus |
| Togaviridae | Alphavirus | | Sindbis virus |

The genome of a +sense ssRNA virus comprises an ssRNA molecule in the 5′-3′ orientation and can be directly translated into the viral proteins by the host cell. Therefore, self-replicating polynucleotides encoding +sense ssRNA viruses do not require the presence of any additional viral replication proteins in order to produce an infectious virus.

In some embodiments, the +sense ssRNA replication-competent viral genomes encoded by the polynucleotides described herein require discrete 5′ and 3′ ends that are native to the virus. mRNA transcripts produced by mammalian RNA Pol II contain mammalian 5′ and 3′ UTRs and therefore do not contain the discrete, native ends required for production of an infectious ssRNA virus. Therefore, in some embodiments, production of infectious +sense ssRNA viruses (e.g., a virus shown in Table 5) requires additional 5′ and 3′ sequences that enable cleavage of the Pol II-encoded viral genome transcript at the junction of the viral ssRNA and the mammalian mRNA sequence such that the non-viral RNA is removed from the transcript in order to maintain the endogenous 5′ and 3′ discrete ends of the virus. Such sequences are referred to herein as junctional cleavage sequences. For example, in some embodiments, the self-polynucleotides comprise the following structure:

(a) 5′-Pol II-junctional cleavage-sense viral genome-junctional cleavage-3′;

(b) 3′-Pol II-junctional cleavage-anti-sense viral genome-junctional cleavage-5′.

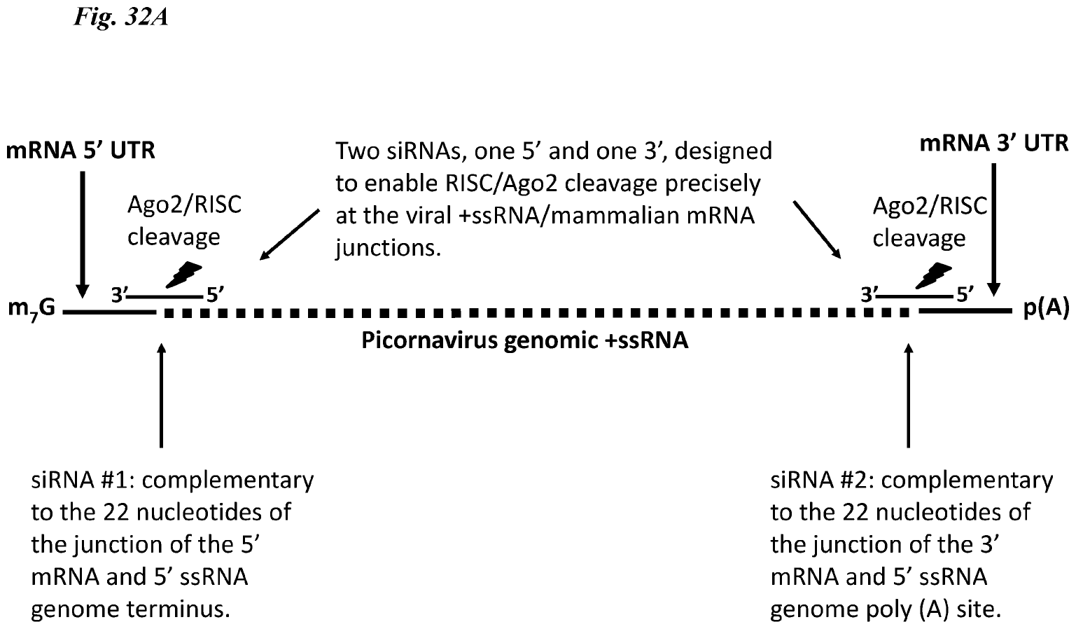

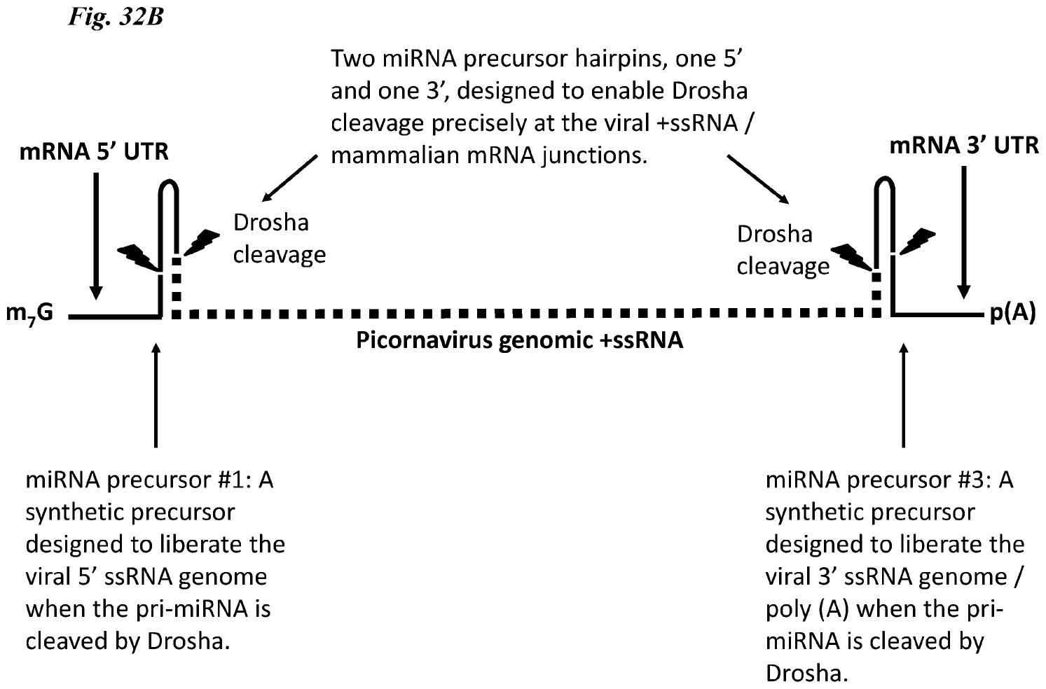

The junctional cleavage sequences and the removal of the non-viral RNA from the viral genome transcript can be accomplished by a variety of methods. For example, in some embodiments, the junctional cleavage sequences are siRNA target sequences and are incorporated into the 5′ and 3′ ends of the self-replicating polynucleotide. In such embodiments, siRNAs can be generated to mediate cleavage of the viral genome transcript by the RNA-induced silencing complex (RISC) or Argonaute proteins. Exemplary construct designs are depicted in FIG. 32A and FIG. 32B. In some embodiments, the junctional cleavage sequences are sequences encoding precursor miRNAs (pri-miRNAs) and are incorporated into the 5′ and 3′ ends of the self-replicating polynucleotide. In such embodiments, the pri-miRNA sequences form hairpin loops that enable cleavage of the viral genome transcript by Drosha. In some embodiments, the junctional cleavage sequences are guide RNA target sequences and are incorporated into the 5′ and 3′ ends of the self-replicating polynucleotide. In such embodiments, gRNAs can be designed and introduced with a Cas endonuclease with RNase activity to mediate cleavage of the viral genome transcript at the precise junctional site. In some embodiments, the junctional cleavage sequences are ribozyme-encoding sequences and are incorporated into the self-replicating polynucleotides described herein immediately 5′ and 3′ of the polynucleotide sequence encoding the viral genome. The encoding ribozymes then mediate cleavage of the viral genome transcript to produce the native discrete ends of the virus. Further, any system for cleaving an RNA transcript at a specific site currently known the art or to be defined the future can be used to generate the discrete ends native to the virus encoded by the self-replicating polynucleotides described herein.

In some embodiments, the self-replicating polynucleotides comprise a 5′ and 3′ junctional cleavage sequence for producing the native discrete ends of the viral transcript, and are flanked by a 5′ and a 3′ ITR. For example, in some embodiments, the self-polynucleotides comprise the following structure:

(a) 5′-ITR-Pol II-junctional cleavage-sense viral genome-junctional cleavage-ITR-3′; or

(b) 3′-ITR-Pol II-junctional cleavage-anti-sense viral genome-junctional cleavage-ITR-5′.

In some embodiments, the polynucleotides comprise the following structure:

(a) 5′-Pol II-ribozyme-sense viral genome-ribozyme-3′;

(b) 3′-Pol II-ribozyme-anti-sense viral genome-ribozyme-5′;

(c) 5′-ITR-Pol II-ribozyme-sense viral genome-ribozyme-ITR-3′; or

(d) 3′-ITR-Pol II-ribozyme-anti-sense viral genome-ribozyme-ITR-5′.

In some embodiments, the 3′ ribozyme-encoding sequence and the 5′ ribozyme-encoding sequence encode the same ribozyme. In some embodiments, the ribozyme-encoding sequences encode a Hepatitis Delta virus ribozyme or a Hammerhead ribozyme. In some embodiments, the 3′ ribozyme-encoding sequence and the 5′ ribozyme-encoding sequence encode different ribozymes. In some embodiments, the 3′ ribozyme-encoding sequence encodes a Hepatitis Delta virus ribozyme and the 5′ ribozyme-encoding sequence encodes a Hammerhead ribozyme.

2. Negative-Sense ssRNA Viruses

In some embodiments, the polynucleotide encodes a negative-sense, single-stranded RNA (−sense ssRNA) viral genome. The genome of a −sense ssRNA virus comprises an ssRNA molecule in the 3′-5′ orientation and cannot be directly translated into protein. Rather, the genome of a −sense ssRNA virus must first be transcribed into a +sense mRNA molecule by an RNA polymerase. Exemplary −sense ssRNA viruses include members of the Paramyxoviridae family (e.g., measles virus and Newcastle Disease virus), the Rhabdoviridae family (e.g., vesicular stomatitis virus (VSV) and marba virus), the Arenaviridae family (e.g., Lassa virus), and the Orthomyxoviridae family (e.g., influenza viruses such as influenza A, influenza B, influenza C, and influenza D).

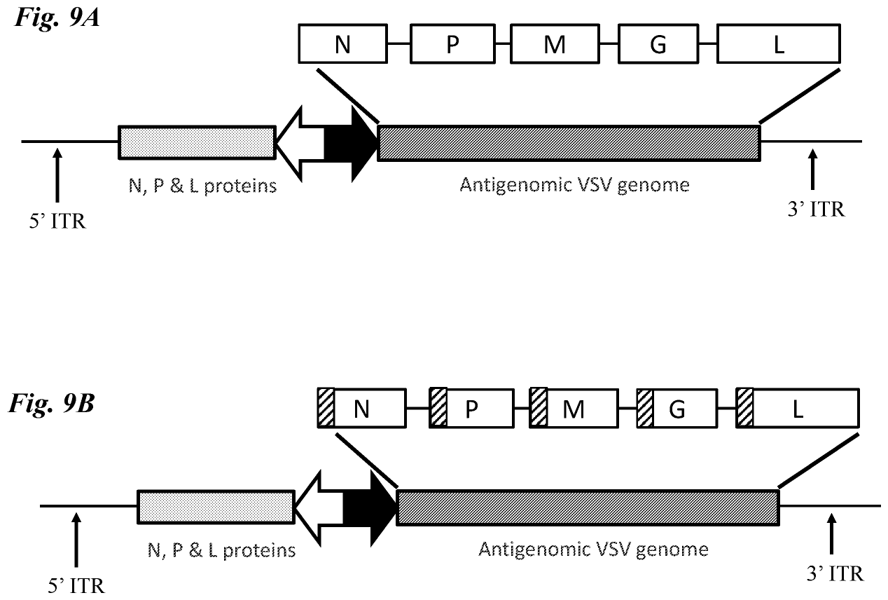

In some embodiments, a self-replicating polynucleotide encoding a −sense ssRNA viral genome comprises a first polynucleotide sequence encoding an mRNA transcript that can be directly translated into the viral proteins required for replication of the −sense ssRNA genome and a second polynucleotide sequence comprising the anti-genomic sequence of the viral genome. In some embodiments, the first and second polynucleotide sequences are operably linked to a promoter capable of expression in eukaryotic cells, e.g. a mammalian promoter. In some embodiments, the first and second polynucleotide sequences are operably linked to a bidirectional promoter, such as a bi-directional Pol II promoter (See e.g., FIGS. 9, 10, and 11).

In some embodiments, the viral genes required for replication of the −sense ssRNA genome are expressed from the same expression cassette. In some embodiments, the viral genes required for replication of the −sense ssRNA genome are expressed from different expression cassettes, e.g., two or three expression cassettes, e.g. an expression cassette for each gene, or one expression cassette with two of the three genes and another with the third gene. The viral genes required for replication of the −sense ssRNA genome may be translated from the same open reading frame or from two or three different open reading frames. In an embodiment, the viral genes required for replication of the −sense ssRNA genome are expressed co-translationally from a single open reading frame and post-translationally processed into mature polypeptides. In an embodiment the viral genes required for replication of the −sense ssRNA genome are linked by 2A peptide sequences, resulting in self-cleavage of the polypeptide translated from the open reading frame into individual polypeptides. The viral genes required for replication of the −sense ssRNA genome genes may be arranged in any order. In some embodiments, the expression cassette comprises functional variants one or more of the viral genes required for replication of the −sense ssRNA genome. Those of skill in the art will recognized how to engineer appropriate variants of the foregoing systems according to the genetic elements needed for a particular −sense ssRNA virus. This engineering may take the form of adding additional genes essential for replication.

In some embodiments, the first polynucleotide sequence encoding an mRNA transcript that can be directly translated into the viral proteins required for replication is operably linked to a promoter capable of expression in a eukaryotic cells, e.g. a mammalian Pol II promoter, and further encodes for a T7 polymerase. In such embodiments, the second polynucleotide sequence is operably linked to a T7 promoter. For example, in some embodiments the self-replicating polynucleotides comprise the following structure:

(a) 5′-[viral genes required for replication]-bi-directional promoter-[anti-genomic viral genome]-3′;

(b) 5′-Pol II-[viral genes required for replication+T7 pol]-T7 promoter-[anti-genomic viral genome]-3′.

In some embodiments, the self-replicating polynucleotide encoding a −sense ssRNA viral genome are flanked on the 5′ and 3′ ends by AAV-derived ITRs, for example:

(a) 5′-ITR-[viral genes required for replication]-bi-directional promoter-[anti-genomic viral genome]-ITR-3′;

(b) 5′-ITR-Pol II-[viral genes required for replication+T7 pol]-T7 promoter-[anti-genomic viral genome]-ITR-3′.

B. Double Stranded RNA Viruses

In some embodiments, the self-replicating polynucleotides described herein encode a double-stranded RNA (dsRNA) viral genome. Exemplary dsRNA viruses include members of the Amalgaviridae family, the Birnaviridae family, the Chrysoviridae family, the Cystoviridae family, the Endornaviridae family, the Hypoviridae family, the Megabirnaviridae family, the Partitiviridae family, the Picobirnaviridae family, the Quadriviridae family, the Reoviridae family, the Totiviridae family.

In some embodiments, the self-replicating polynucleotides described herein encode dsRNA viral genomes. In some embodiments, the dsRNA viral genome is encoded as a positive sense strand 5′ to a negative sense (complementary) strand. Thus, in some embodiments, the dsRNA viral genome is transcribed as two RNA molecules that are complementary to another from the same strand of the DNA polynucleotide. In some embodiments, the two RNA molecules of the dsRNA viral genome are transcribed as a single RNA, which is cleaved into positive and negative sense molecules, e.g. by a ribozyme, endonuclease, CRISPR-based system, or the like.

In an embodiment, the dsRNA viral genome is transcribed from a shared dsDNA template operatively linked to promoters flanking the shared dsDNA template. One promoter causes transcription from the Watson strand of the DNA polynucleotide, thereby generating the positive strand of the dsRNA genome. The other promoter causes transcription from the Crick strand of the DNA polynucleotide, thereby generating the negative strand of the dsRNA genome. Some dsRNA virus, e.g. reovirus, are segmented viruses, meaning that their genomes are comprised of multiple RNA molecules, in some cases a mixture of dsRNA and ssRNA. The disclosure provides embodiments in which the DNA polynucleotide comprises transcriptional units for each of the segments. In some embodiments, the segments are transcribed from several promoters on the Watson and/or Crick strands of the DNA polynucleotide. In some embodiments, the RNA segments are generated by post-transcriptional cleavage of one or more RNA segments, e.g. by a ribozyme, endonuclease, CRISPR-based system, or the like. In some embodiments, one or more of the promoters of the system is a T7 promoter and the system comprises a polynucleotide encoding a T7 RNA polymerase. In some embodiments, use of a T7 system generates a native 5′ termini for one or more segments of the dsRNA viral genome. In some embodiments, one or more of the promoters of the system is a eukaryotically active promoter, e.g. a mammalian promoter.

C. Single-Stranded DNA Viruses

In some embodiments, the self-replicating polynucleotides described herein encode a single-stranded DNA (ssDNA) viral genome. Exemplary ssDNA viruses include members of the Parvoviridae family (e.g., adeno-associated viruses), the Anelloviridae family, the Bidnaviridae family, the Circoviridae family, the Geminiviridae family, the Genomoviridae family, the Inoviridae family, the Microviridae family, the Nanoviridae family, the Smacoviridae family, and the Spiraviridae family. In an embodiment, the self-replicating polynucleotides encodes a parvovirus. In an embodiment, the self-replicating polynucleotides encodes an adeno-associated virus (AAV).

D. Double-Stranded DNA Viruses

In some embodiments, the self-replicating polynucleotides described herein encode a double-stranded DNA (dsDNA) viral genome. Exemplary dsDNA viruses include members of the Myoviridae family, the Podoviridae family, the Siphoviridae family, the Alloherpesviridae family, the Herpesviridae family (e.g., HSV-1, HSV-1, Equine Herpes Virus), the Poxviridae family (e.g., vaccina virus and myxoma virus). In an embodiment, the self-replicating polynucleotides encodes an adenovirus.

E. miRNA-Attenuation

In some embodiments, the self-replicating polynucleotides described herein encode a replication-competent viral genome comprising one or more micro RNA (miRNA) target sequences inserted into one or more essential viral genes. miRs regulate many transcripts encoding numerous proteins, including those involved in the control of cellular proliferation and apoptosis. Exemplary proteins that are regulated by miRs include conventional proto-oncoproteins and tumor suppressors such as Ras, Myc, Bcl2, PTEN and p53.

miRNAs are intimately associated with normal cellular processes and their dysregulation contributes to a wide array of diseases including cancer. Importantly, miRNAs are differentially expressed in cancer tissues compared to normal tissues, enabling them to serve as a targeting mechanism in a broad variety of cancers. miRNAs that are associated (either positively or negatively) with carcinogenesis, malignant transformation, or metastasis are known as “oncomiRs”. Table 2 provides a list of oncomiRs and their relative expression in particular cancers.

In some aspects, the expression of a particular miRNA is positively associated with the development or maintenance of a particular cancer and/or metastasis. Such miRs are referred to herein as “oncogenic miRNAs” or “oncomiRs.” In some embodiments, the expression of an oncogenic miRNA is increased in cancerous cells or tissues compared to the expression level observed in non-cancerous control cells (i.e., normal or healthy controls), or is increased compared to the expression level observed in cancerous cells derived from a different cancer type. For example, the expression of an oncogenic miRNA in a cancerous cell may be increased by at least 5%, 10%, 15%, 20%, 25%, 30%, 35%, 40%, 45%, 50%, 60%, 70%, 80%, 90%, 100%, 150%, 200%, 300%, 400%, 500%, 1000% or more compared to the expression of the oncogenic miRNA in a non-cancerous control cell or a cancerous cell derived from a different cancer type. In some aspects, a cancerous cell may express an oncogenic miRNA that is not expressed in non-cancerous control cells.

In some embodiments, the expression of a particular oncomiR is negatively associated with the development or maintenance of a particular cancer and/or metastasis. Such oncomiRs are referred to herein as “tumor-suppressor miRNAs” or “tumor-suppressive miRNAs,” as their expression prevents or suppresses the development of cancer. In some embodiments, the expression of a tumor-suppressor miRNA is decreased in cancerous cells or tissues compared to the expression level observed in non-cancerous control cells (i.e., normal or healthy controls), or is decreased compared to the expression level of the tumor-suppressor miRNA observed in cancerous cells derived from a different cancer type. For example, the expression of a tumor-suppressor miRNA in a cancerous cell may be decreased by at least 5%, 10%, 15%, 20%, 25%, 30%, 35%, 40%, 45%, 50%, 60%, 70%, 80%, 90%, or 100% compared to the expression of the tumor-suppressor miRNA in a non-cancerous control cell or a cancerous cell derived from a different cancer type. In some aspects, a non-cancerous control cell may express a tumor-suppressor miRNA that is not expressed in cancerous cells.

Typically, the designation of a particular miRNA as an oncogenic vs. a tumor suppressive miRNA will vary according to the type of cancer. For example, the expression of one miRNA may be increased in a particular cancer and associated with the development of that cancer, while the expression of the same miRNA may be decreased in a different cancer and associated with prevention of the development of that cancer. However, some miRNAs may function as oncogenic miRNAs independent of the type of cancer. For example, some miRNAs target mRNA transcripts of tumor suppressor genes for degradation, thereby reducing expression of the tumor suppressor protein. Table 2 provides a list of several cancers and the corresponding “up-regulated” miRNAs and “down-regulated” miRNAs observed in each cancer type. In Table 2, the up-regulated miRNAs are miRNAs that are likely oncogenic in that particular cancer, while the down-regulated miRNAs are likely tumor-suppressive in that particular cancer. A list of additional tumor-suppressive miRNAs is shown in Table 3. Table 1 shows the relationship between 12 select oncomiRs (9 tumor suppressors and 3 oncogenic miRNAs) and numerous cancers.

In some aspects, the replication of a virus produced by the polynucleotides described herein is restricted to tumor cells by incorporation of one or more miRNA target sequences at one or more locations in the viral genome. In some embodiments, the one or more miRNA target sequences are incorporated into the 5′ UTR and/or the 3′ UTR of the replication competent viral genome. In some embodiments, the one or more miRNA target sequences are incorporated into one or more loci of essential viral genes. As used herein, “essential viral genes” refers to viral genes that are required for viral replication, assembly of viral gene products into an infectious particle, or are required to maintain the structural integrity of the assembled infectious particle. In some embodiments, essential viral genes may include UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL50, UL52, UL53, UL54, US1, US3, US4, US5, US6, US7, US8, US12, ICP0, ICP4, ICP22, ICP27, ICP47, PB, F, B5R, SERO-1, Cap, Rev, VP1-4, nucleoprotein (N), phosphoprotein (P), matrix protein (M), glycoprotein (G), polymerase (L), E1, E2, E3, E4, VP1, VP2, VP3, VP4, 2A, 2B, 2C, 3A, 3B, 3C, and 3D.

In some embodiments, the miRNA target sequences inserted into one or more loci of essential viral genes correspond to miRNAs that are expressed by normal, non-cancerous cells and that are not expressed or demonstrate reduced expression in cancerous cells. A miRNA expressed in normal (non-cancerous) cells will bind to the corresponding target sequence in the polynucleotide and suppress expression of the viral gene containing the miRNA target sequence, thereby preventing viral replication and/or structural assembly into an infectious particle. Thus, the insertion of the miRNA target sequences protects normal cells from lytic effects of the encoded virus. In some embodiments, the miRNA target sequences are target sequences for tumor-suppressive miRNAs (e.g., a miRNA listed in Table 3). In some embodiments, a polynucleotide may comprise a miRNA target sequence inserted into a locus of at least one, at least two, at least three, at least four, at least five, at least six, at least seven, at least eight, at least nine, or at least ten essential viral genes. In some embodiments, the one or more miRNA target sequences is incorporated into the 5′ untranslated region (UTR) and/or 3′ UTR of one or more essential viral genes. In some embodiments, the one or more miRNA target sequences is incorporated into the 3′ or 5′ UTR of a non-essential gene in a viral genome (e.g., gamma 34.5).

In some embodiments, the polynucleotides described herein comprise a miRNA target sequence incorporated into a loci of an essential viral gene. In some aspects, the self-replicating polynucleotides described herein comprise a plurality of miRNA target sequences incorporated into one or more essential viral genes. In some embodiments, the polynucleotides comprise a miRNA target sequence incorporated into a plurality (e.g., 2 or more) of essential viral genes. For example, the polynucleotides described herein may comprise a miRNA target sequence inserted into 2, 3, 4, 5, 6, 7, 8, 9, 10 or more essential viral genes. In such embodiments, each essential viral gene would comprise one miRNA target sequence, while the polynucleotide as a whole would comprise a plurality of miRNA target sequences. In such embodiments, the plurality of miRNA target sequences may correspond to the same miRNA. For example, the polynucleotides described herein may comprise the same miRNA target sequence inserted into 2, 3, 4, 5, 6, 7, 8, 9, 10 or more essential viral genes. In such embodiments, the plurality of miRNA target sequences may correspond to two or more different miRNAs. For example, the polynucleotides described herein may comprise a miRNA target sequence corresponding to a first miRNA inserted into a first essential viral gene, a miRNA target sequence corresponding to a second miRNA inserted into a second essential viral gene, a miRNA target sequence corresponding to a third miRNA inserted into a third essential viral gene, and so on.

In some embodiments, a plurality of copies of a miRNA target sequence are incorporated into a locus of an essential viral gene. For example, in some embodiments, 2, 3, 4, 5, 6, 7, 8, 9, 10, or more copies of a miRNA target sequence can be inserted into a locus of an essential viral gene. In some embodiments, each of the plurality miRNA target sequences inserted into the loci of the essential viral gene corresponds to the same miRNA. In some embodiments, each of the plurality of miRNA target sequences inserted into a loci of an essential viral gene corresponds to a different miRNA. For example, miRNA target sequences corresponding to 2, 3, 4, 5, 6, 7, 8, 9, 10 or more different miRNAs can be inserted into a loci of an essential viral gene.

In some embodiments, a plurality of copies of a miRNA target sequence are incorporated into a locus of a plurality of essential viral genes. For example, in some embodiments, 2, 3, 4, 5, 6, 7, 8, 9, 10, or more copies of a miRNA target sequence can be inserted into a locus of 2, 3, 4, 5, 6, 7, 8, 9, 10, or more essential viral genes. In some embodiments, the plurality of miRNA target sequences inserted into a particular essential viral gene may all correspond to the same miRNA. For example, in some embodiments, a first essential viral gene may comprise a plurality of miRNA target sequences each corresponding to a first miRNA and a second essential viral gene may comprise a plurality of miRNA target sequences each corresponding to a second miRNA. In some embodiments, the self-replicating polynucleotides may further comprise a third, fourth, fifth, sixth, seventh, eighth, ninth, or tenth essential viral gene comprising a plurality of miRNA target sequences each corresponding to a third, fourth, fifth, sixth, seventh, eighth, ninth, or tenth miRNA, respectively.

In some embodiments, a plurality of miRNA target sequences corresponding to different miRNAs are inserted into a plurality of essential viral gene loci. For example, in some embodiments, a first essential viral gene may comprise a plurality of miRNA target sequences corresponding to two or more different miRNAs and a second essential viral gene may comprise a plurality of miRNA target sequences corresponding to two or more different miRNAs. In such embodiments, the miRNA target sequences in the first essential viral gene may be the same or different than the miRNA target sequences in the second essential viral gene. In some embodiments, the self-replicating polynucleotides may further comprise a third, fourth, fifth, sixth, seventh, eighth, ninth, or tenth essential viral gene, each comprising a plurality of miRNA target sequences corresponding to different miRNAs. In some embodiments, the miRNA target sequences in any one of the first, second, third, fourth, fifth, sixth, seventh, eighth, ninth, or tenth essential viral genes may be the same as the miRNA target sequences in any of the other essential viral genes. In some embodiments, the miRNA target sequences in any one of the first, second, third, fourth, fifth, sixth, seventh, eighth, ninth, or tenth essential viral genes may be different than the miRNA target sequences in any of the other essential viral genes.

In some embodiments, a plurality of miRNA target sequences are inserted in tandem into a locus of one or more essential viral genes and are separated from each other by a linker sequence or a spacer sequence. In some embodiments, the linker or spacer space sequence comprises 4 or more nucleotides. In some embodiments, the linker or spacer space sequence comprises 5, 6, 7, 8, 9, 10, or more nucleotides. In one embodiment, the linker sequence or the spacer sequence comprises at least 4 to at least 6 nucleotides.

In some embodiments, at least 1, 2, 3, 4, 5, 6, 7, 8, 9, 10 or more of either of the following subunits inserted in tandem into a locus of one or more essential viral genes: (a) target sequence for a first miRNA-linker or spacer sequence-target sequence for the first miRNA; or (b) target sequence for a first miRNA-linker or spacer sequence-target sequence for a second miRNA. In some embodiments, the miRNA target sequences are target sequences for any one or more of the miRNAs listed in Table 3.

F. Payload Molecules

In some embodiments, the polynucleotides described herein comprise a nucleic acid sequence encoding a payload molecule. As used herein, a “payload molecule” (also referred to as a “therapeutic molecule”) refers to any molecule capable of further enhancing the therapeutic efficacy of a virus encoded by a self-replicating polynucleotide described herein or infectious particles thereof. Payload molecules suitable for use in the present disclosure include proteins or peptides such as cytotoxic peptides, immune modulatory peptides (e.g., antigen-binding molecules such as antibodies or antigen binding fragments thereof, cytokines, chemokines, soluble receptors, cell-surface receptor ligands, bipartite peptides, and enzymes. Such payload molecules may also comprise nucleic acids (e.g., shRNAs, siRNAs, antisense RNAs, antagomirs, ribozymes, and apatamers). The nature of the payload molecule will vary with the disease type and desired therapeutic outcome.

In some embodiments, one or more miRNA target sequences is incorporated in to the 3′ or 5′ UTR of a polynucleotide sequence encoding a payload molecule. In such embodiments, translation and subsequent expression of the payload does not occur, or is substantially reduced, in cells where the corresponding miRNA is expressed. In some embodiments, one or more miRNA target sequences are inserted into the 3′ and/or 5′ UTR of the polynucleotide sequence encoding the therapeutic polypeptide.

In some embodiments, expression of the therapeutic molecules may be further regulated by transcriptional control elements that drive increased expression of the therapeutic molecule in cancer cells compared to non-cancerous cells (e.g., promosters derived from hTERT, HE4, CEA, OC, ARF, CgA, GRP78, CXCR4, HMGB2, INSM1, Mesothelin, OPN, RAD51, TETP, H19, uPAR, ERBB2, MUC1, Frz1, IGF2-P4, or hypoxia (HREs) and radiation responsive elements). In some embodiments, the expression of the payload molecule is under the control of the same transcriptional control element as the self-replicating polynucleotide.

In some embodiments, recombinant nucleic acid molecules described herein comprise a self-replicating polynucleotide and further comprise a polynucleotide encoding a cytotoxic peptide. As used herein, a “cytotoxic peptide” refers to a protein capable of inducing cell death in when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribosome inactivating proteins (RIPs) (e.g., saporin and geionin), Type II RIPs (e.g., ricin), Shiga-like toxin 1 (Slt1), photosensitive reactive oxygen species (e.g. killer-red). In certain embodiments, the cytotoxic peptide is encoded by a suicide gene resulting in cell death through apoptosis, such as a caspase gene.

In some embodiments, the payload is an immune modulatory peptide. As used herein, an “immune modulatory peptide” is a peptide capable of modulating (e.g., activating or inhibiting) a particular immune receptor and/or pathway. In some embodiments, the immune modulatory peptides can act on any mammalian cell including immune cells, tissue cells, and stromal cells. In a preferred embodiment, the immune modulatory peptide acts on an immune cell such as a T cell, an NK cell, an NKT T cell, a B cell, a dendritic cell, a macrophage, a basophil, a mast cell, or an eosinophil. Exemplary immune-modulatory peptides include antigen-binding molecules such as antibodies or antigen binding fragments thereof, cytokines, chemokines, soluble receptors, cell-surface receptor ligands, bipartite peptides, and enzymes.

In some embodiments, recombinant nucleic acid molecules described herein comprise a self-replicating polynucleotide and further comprise a polynucleotide encoding a cytokine such as IL-1, IL-12, IL-15, IL-18, TNFα, IFNα, IFNβ, or IFNγ. In some embodiments, recombinant nucleic acid molecules described herein comprise a self-replicating polynucleotide and further comprise a polynucleotide encoding a chemokine such as CXCL10, CXCL9, CCL21, CCL4, or CCL5. In some embodiments, recombinant nucleic acid molecules described herein comprise a self-replicating polynucleotide and further comprise a polynucleotide encoding a ligand for a cell-surface receptor such as an NKG2D ligand, a neuropilin ligand, Flt3 ligand, a CD47 ligand (e.g., SIRP1α). In some embodiments, recombinant nucleic acid molecules described herein comprise a self-replicating polynucleotide and further comprise a polynucleotide encoding a soluble receptor, such as a soluble cytokine receptor (e.g., IL-13R, TGFβR1, TGFβR2, IL-35R, IL-15R, IL-2R, IL-12R, and interferon receptors) or a soluble innate immune receptor (e.g., toll-like receptors, complement receptors, etc.). In some embodiments, recombinant nucleic acid molecules described herein comprise a self-replicating polynucleotide and further comprise a polynucleotide encoding a dominant agonist mutant of a protein involved in intracellular RNA and/or DNA sensing (e.g. a dominant agonist mutant of STING, RIG-1, or MDA-5).

In some embodiments, recombinant nucleic acid molecules described herein comprise a self-replicating polynucleotide and further comprise a polynucleotide encoding an antigen-binding molecule such as an antibody or antigen binding fragments thereof (e.g., a single chain variable fragment (scFv), an F(ab), etc.). In some embodiments, the antigen-binding molecule specifically binds to a cell surface receptor, such as an immune checkpoint receptor (e.g., PD1, PDL1, and CTLA4) or additional cell surface receptors involved in cell growth and activation (e.g., OX40, CD200R, CD47, CSF1R, 41BB, CD40, and NKG2D).

In some embodiments, the payload molecule is a scorpion polypeptide such as chlorotoxin, BmKn-2, neopladine 1, neopladine 2, and mauriporin. In some embodiments, the therapeutic molecule is a snake polypeptide such as contortrostatin, apoxin-I, bothropstoxin-I, BJcuL, OHAP-1, rhodostomin, drCT-I, CTX-III, B1L, and ACTX-6. In some embodiments, the payload molecule is a spider polypeptide such as a latarcin and hyaluronidase. In some embodiments, the payload molecule is a bee polypeptide such as melittin and apamin. In some embodiments, the payload molecule is a frog polypeptide such as PsT-1, PdT-1, and PdT-2.

In some embodiments, recombinant nucleic acid molecules described herein comprise a self-replicating polynucleotide and further comprise a polynucleotide encoding an enzyme. In some embodiments, the enzyme is capable of modulating the tumor microenvironment by way of altering the extracellular matrix. In such embodiments, the enzyme may include, but is not limited to, a matrix metalloprotease (e.g., MMP9), a collagenase, a hyaluronidase, a gelatinase, or an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450. In some embodiments, the enzyme is capable of inducing or activating cell death pathways in the target cell (e.g., a caspase).

In some embodiments, the payload molecule is a bipartite peptide. As used herein, a “bipartite peptide” refers to a multimeric protein comprised of a first domain capable of binding a cell surface antigen expressed on a non-cancerous effector cell and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g, a single chain variable fragment (scFv) or an F(ab)) a scorpion polypeptide, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-Tg™), a Tandab®, a bi-specific T cell engager (BiTE™), a DuoBody®, or a dual affinity retargeting (DART) polypeptide. In some embodiments, the bipartite polypeptide is a BiTE and comprises a domain that specifically binds to an antigen shown in Table 6 and/or 7. Exemplary BiTEs are shown below in Table 5.

TABLE 5
Validated BiTEs used in preclinical and clinical studies

| Target | Name | Target Disease | Clinical Status | References |
|---|---|---|---|---|
| CD19 | Blinatumomab/MT- | NHL, ALL | Phase I/II/III | 1, 2, 3, 4, 5, 6 |
| | 103/MEDI-538 | | | |
| EpCAM | MT110 | Solid tumors | Phase I | 7, 8, 9, 10 |
| CEA | MT111/MEDI-565 | GI adenocarcinoma | Phase I | 11, 12 |
| PSMA | BAY2010112/AMG112 | Prostate | Phase I | 13 |
| CD33 | AMG330 | AML | Preclinical | 14, 15 |
| EGFR | C-BiTE and P-BiTE | Colorectal cancer | Preclinical | 16 |
| | antibodies | | | |
| Her2 | FynomAb, | Breast and gastric | Preclinical | 17, 18 |
| | COVA420, HER2- | carcinoma | | |
| | BsAb | | | |
| EphA2 | bscEphA2xCD3 | Multiple solid | Preclinical | 19 |
| | | tumors | | |
| MCSP | MCSP-BiTE | Melanoma | Preclinical | 20 |
| ADAM17 | A300E | Prostate cancer | Preclinical | 21 |
| PSCA | CD3-PSCA(MB1) | Prostate cancer | Preclinical | 22 |
| 17-A1 | CD3/17-1A-bispecific | Colorectal cancer | Preclinical | 23 |
| NKG2D | scFv-NKG2D, | Multiple solid and | Preclinical | 24, 25 |
| ligands | huNKG2D-OKT3 | liquid tumors | | |
| DLL3 | AMG757 | Small Cell Lung | Clinical | 26 |
| | | Cancer | | |

In some embodiments, the cell-surface antigen expressed on an effector cell is selected from Table 6 below. In some embodiments, the cell-surface antigen expressed on a tumor cell or effector cell is selected from Table 7 below. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, or neuropilin. In some embodiments, the tumor antigen is selected from those listed in Table 7.

TABLE 6
Exemplary effector cell target antigens

| T cell | | NKT cell | NK Cell | Other |
|---|---|---|---|---|
| CD3 | CD30 | CD3 | CD16 | CD48 |
| CD3γ | CD38 | CD3γ | CD94/NKG2 | LIGHT |
| | | | (e.g., NKG2D) | |
| CD3δ | CD40 | CD3δ | NKp30 | CD44 |
| CD3ε | CD57 | CD3ε | NKp44 | CD45 |
| CD3ξ | CD69 | CD3ξ | NKp46 | IL-1R2 |
| CD2 | CD70 | invariant TCR | KARs | IL-1Rα |
| CD4 | CD73 | | | IL-1Rα2 |
| CD5 | CD81 | | | IL-13Rα2 |
| CD6 | CD82 | | | IL-15Ra |
| CD7 | CD96 | | | CCR5 |
| CD8 | CD134 | | | CCR8 |
| CD16 | CD137 | | | |
| CD25 | CD152 | | | |
| CD27 | CD278 | | | |
| CD28 | | | | |

TABLE 7
Exemplary target cell antigens
Target Cell Antigens

| | | | |
|---|---|---|---|
| 8H9 | CRISP3 | Lewis-Y | SOX2 |
| GnT-V, β1, 6-N | DC-SIGN | LIV-1 | STEAP1 |
| AFP | DHFR | Livin | SLITRK6 |
| ART1 | EGP40 | LAMP1 | NaPi2a |
| ART4 | EZH2 | MAGEA3 | SOX1 |
| ABCG2 | EpCAM | MAGEA4 | SOX11 |
| B7-H3 | EphA2 | MAGEB6 | SPANXA1 |
| B7-H4 | EphA2/Eck | MAGEA1 | SART-1 |
| B7-H6 | EGFRvIII | MART-1 | SSX4 |
| BCMA | E-cadherin | MCSP | SSX5 |
| B-cyclin | EGP2 | MME | Survivin |
| BMI1 | ETA | mesothelin | SSX2 |
| CA-125 | ERBB3 | MAPK1 | TAG72 |
| cadherin | ERBB3/4 | MUC16 | TEM1 |
| CABYR | ERBB4 | MUC1 | TEM8 |
| CTAG2 | EPO | MRP-3 | TSGA10 |
| CA6 | FAR | MyoD-1 | TSSK6 |
| CAIX | FBP | NCAM | thyroglobulin |
| CEA | FTHL17 | nectin 4 | transferrin receptor |
| CEACAM5 | fetal AchR | Nestin | TMEM97 |
| Cav-1 | FAP | NEP | TRP-2 |
| CD10 | FGFR3 | NY-ESO-1 | TULP2 |
| CD117 | FR-a | hHLA-A | TROP2 |
| CD123 | Fra-1/Fosl 1 | H60 | tyrosinase |
| CD133 | GAGE1 | OLIG2 | TRP1 |
| CD138 | GD2 | 5T4 | UPAR |
| CD15 | GD3 | p53 | VEGF |
| CD171 | Glil | P-Cadherin | VEGF receptors |
| CD19 | GP100 | PB | VEGRR2 |
| CD20 | GPA33 | P-glycoprotein | BRAF |
| CD21 | Glypican-3 | PRAME | WT-1 |
| CD22 | HIV gp120 | PROX1 | XAGE2 |
| CD30 | HLA-A | PSA | ZNF165 |
| CD33 | HLA-A2 | PSCA | αᵥβ₆ integrin |
| CD38 | HLA-AI | PSMA | β-catenin |
| CD44v6 | HLA-B | PSC1 | cathepsin B |
| CD44v7/8 | HLA-C | Ras | CSAG2 |
| CD74 | HMW-MAA | ROR1 | CTAG2 |
| Cd79b | Her2/Neu | SART2 | EGFR |
| Ki-67 | u70/80 | SART3 | EGP40 |
| CSPG4 | LICAM | oncofetal variants | EZH2 |
| | | of fibronectin | |
| CALLA | ULBP1 | tenascin | HIV sp120 |
| CSAG2 | ULBP2 | LICAM | kappa light chain |
| COX-2 | ULBP3 | Rae-1α | LDHC |
| Lambda | MICA | Rae-1β | TRP-1 |
| LAYN | MICB | Rae-1δ | Fas-L |
| LeuM-1 | Her3 | Rae-1γ | |
| KDR | EGF | PDGF | |
| CD47 | SIRP 1α | Fas | DLL3 |

### III. Methods of Producing Recombinant Nucleic Acid Molecules Comprising Self-Replicating Polynucleotides

In some embodiments, the recombinant nucleic acid molecules described herein are produced in vitro using one or more vectors. The term “vector” is used herein to refer to a nucleic acid molecule capable transferring or transporting another nucleic acid molecule. The transferred nucleic acid is generally inserted into the vector nucleic acid molecule. A vector may include sequences that direct autonomous replication in a cell and/or may include sequences sufficient to allow integration into host cell DNA.

In some embodiments, the recombinant nucleic acid molecules described herein are produced by insertion of a self-replicating polynucleotide described herein into a plasmid backbone.

In some embodiments, the recombinant nucleic acid molecules described herein are produced using one or more viral vectors. A viral vector may sometimes be referred to as a “recombinant virus” or a “virus.” In some embodiments, a two-vector system is used. For example, in some embodiments, the self-replicating polynucleotides described herein are flanked by AAV-derived ITRs. The ITR-flanked polynucleotide is then inserted into a first expression vector and a polynucleotide encoding AAV proteins that are required for ITR-mediated replication (e.g., Rep78 and Rep52) are inserted into a second expression vector. In such embodiments, the first and second vectors are delivered intracellularly (e.g., by means of transfection, transduction, electroporation, and the like) to a suitable host cell (e.g., an insect cell line), to produce a cell wherein the ITR-flanked polynucleotide is stably integrated into the host cell's genome. In some embodiments, the first and second vectors are herpes virus expression vectors. In some embodiments, the first and second vectors are baculovirus expression vectors. Such expression systems are described, for example, in Li et al., Plos One, 8:8, 2013. In some embodiments, the host cell produces the ITR-flanked self-replicating polynucleotide in amounts greater than amounts produced in the absence of ITRs. In some embodiments, ITR-flanked viral genome DNA from host cells transfected with ITR-flanked transgenes may produce 4 to 60-fold more DNA than similarly transfected transgenes that do not contain ITRs (e.g. via recombinant baculovirus infection) (See, Li et al, PLoS One, 2013).

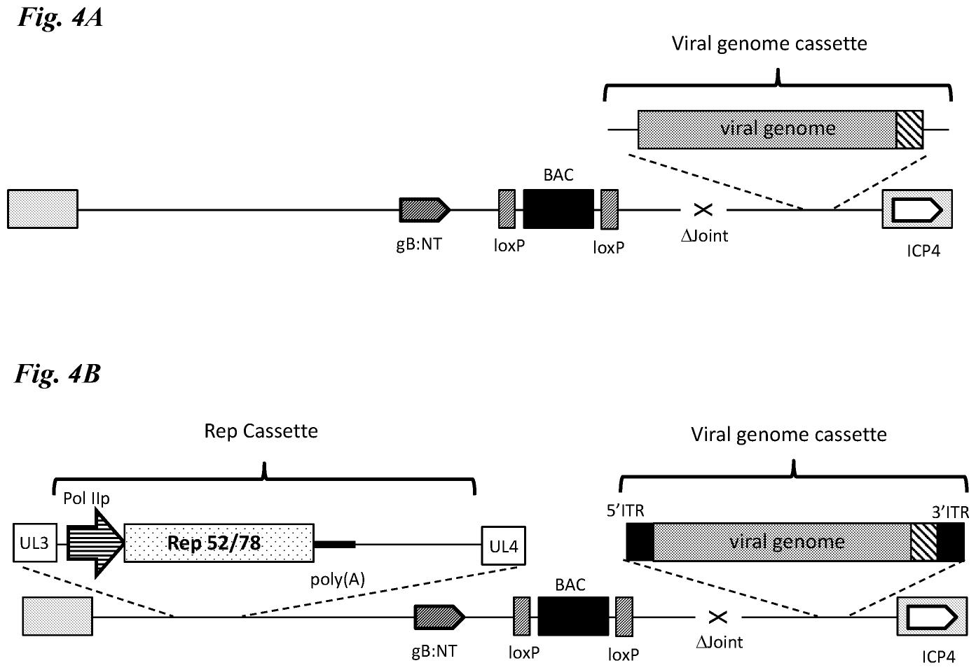

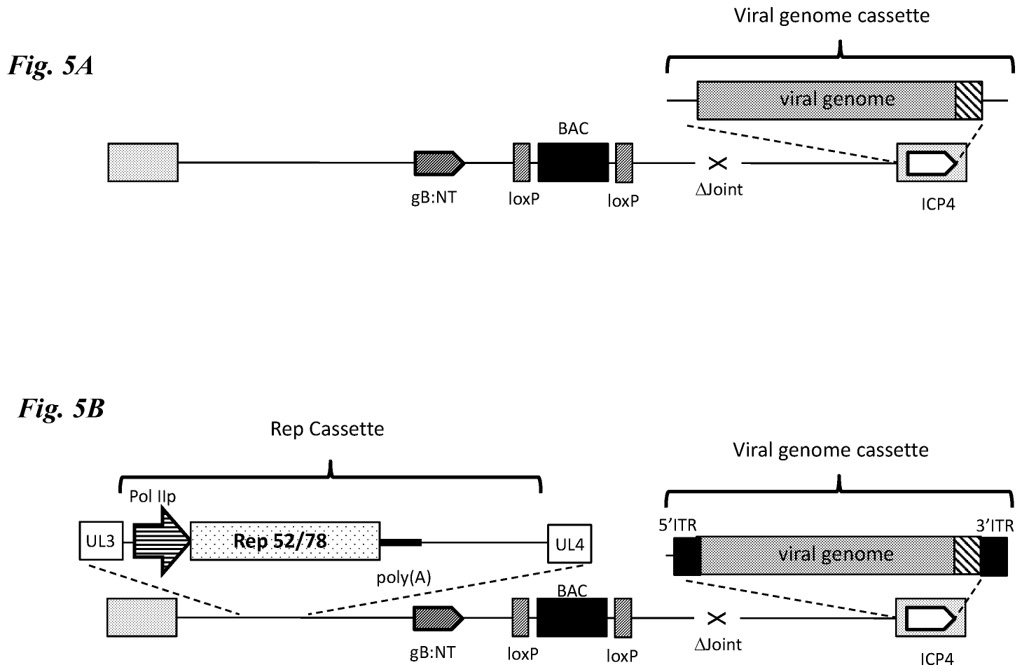

In some embodiments, the polynucleotides described herein are produced in vitro using a single-vector expression system. For example, in some embodiments, an expression cassette comprising the self-replicating polynucleotides described herein flanked by AAV ITRs is inserted between the UL3 and UL4 genes (e.g. into an intergenic locus) or ICP4 locus of a recombinant HSV genome backbone (See e.g., FIG. 4B and FIG. 5B). A second expression cassette comprising Polynucleotides encoding AAV proteins that are required for ITR-mediated replication (e.g., Rep78 and Rep52) is inserted into the ICP0 or ICP4 locus of the recombinant HSV genome backbone. Expression of the Rep proteins enables efficient replication of ITR-flanked polynucleotide from a single vector. In some embodiments, the polynucleotides encoding the Rep proteins are operably linked to a regulatable or inducible promoter.

In some embodiments, the recombinant nucleic acid molecules described herein are produced by intracellularly (e.g., by means of transfection, transduction, electroporation, and the like) to a suitable host cell an HSV vector comprising an expression cassette comprising an ITR-flanked self-replicating polynucleotide and an expression cassette comprising polynucleotides encoding AAV proteins required for ITR-mediated replication. Suitable host cells include insect and mammalian cell lines. Host-cells comprising the HSV vectors are cultured for an appropriate amount of time allow expression of the inserted expression cassettes and production of the recombinant DNA molecules. The recombinant DNA molecules are then isolated from the host cell DNA and formulated for therapeutic use (e.g., encapsulated in a particle).

In some embodiments, the recombinant DNA molecules produced by the AAV-ITR systems described above result in the production of two single stranded DNA molecules covalently linked together at each terminus. For example, the 5′ ITR of the first DNA molecule is covalently linked to the 3′ ITR of the second DNA molecule and the 3′ ITR of the first DNA molecule is covalently linked to the 5′ ITR of the second DNA molecule. In such embodiments, the covalently linked ITR-flanked polynucleotides form an end-closed, linear duplexed oncolytic virus nucleic acid molecule, referred to herein as a NanoV molecule. In some embodiments, each of the single stranded DNA molecules comprises a single ITR-flanked polynucleotide. For example, in some embodiments, a NanoV molecule comprises two ssDNA molecules wherein one ssDNA molecule comprises the following structure: 5′-ITR-[sense sequence of self-replicating polynucleotide]-ITR-3′; and wherein one ssDNA molecule comprises the following structure: 3′-ITR-[antisense sequence of self-replicating polynucleotide]-ITR-3′. In some embodiments, each of the single stranded DNA molecules comprises two or more ITR-flanked polynucleotides (i.e., concantamers of the ITR-flanked polynucleotides). The concantamers of the ITR-flanked polynucleotides can have a variety of orientations. For example, in some embodiments, the concantamers are formed in a head-to-head orientation or in a tail-to-tail orientation.

### IV. Particles Comprising Self-Replicating Polynucleotides

In some embodiments, the polynucleotides described herein are encapsulated in “particles.” As used herein, a particle refers to a non-tissue derived composition of matter such as liposomes, lipoplexes, nanoparticles, nanocapsules, microparticles, microspheres, lipid particles, exosomes, vesicles, and the like. In certain embodiments, the particles are non-proteinaceous and non-immunogenic. In such embodiments, encapsulation of the polynucleotides described herein allows for delivery of a viral payload without the induction of a systemic, anti-viral immune response and mitigates the effects of neutralizing anti-viral antibodies. Further, encapsulation of the polynucleotides described herein shields the polynucleotides from degradation, and facilitates the introduction of the polynucleotide into target host cells.

In some embodiments, the particle is biodegradable in a subject. In such embodiments, multiple doses of the particles can be administered to a subject without an accumulation of particles in the subject. Examples of suitable particles include polystyrene particles, poly(lactic-co-glycolic acid) PLGA particles, polypeptide-based cationic polymer particles, cyclodextrin particles, chitosan particles, lipid based particles, poly(β-amino ester) particles, low-molecular-weight polyethylenimine particles, polyphosphoester particles, disulfide cross-linked polymer particles, polyamidoamine particles, polyethylenimine (PEI) particles, and PLURIONICS stabilized polypropylene sulfide particles.

In some embodiments, the polynucleotides described herein are encapsulated in inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticles (SHNP).

A. Exosomes

In some embodiments, the polynucleotides described herein are encapsulated in exosomes. Exosomes are small membrane vesicles of endocytic origin that are released into the extracellular environment following fusion of multivesicular bodies with the plasma membrane of the parental cell (e.g., the cell from which the exosome is released, also referred to herein as a donor cell). The surface of an exosome comprise a lipid bilayer derived from the parental cell's cell membrane and can further comprise membrane proteins expressed on the parental cell surface. In some embodiments, exosomes may also contain cytosol from the parental cell. Exosomes are produced by many different cell types including epithelial cells, B and T lymphocytes, mast cells (MC), and dendritic cells (DC) and have been identified in blood plasma, urine, bronchoalveolar lavage fluid, intestinal epithelial cells, and tumor tissues. Because the composition of an exosome is dependent on the parental cell type from which they are derived, there are no “exosome-specific” proteins. However, many exosomes comprise proteins associated with the intracellular vesicles from which the exosome originated in the parental cells (e.g., proteins associated with and/or expressed by endosomes and lysosomes). For example, exosomes can be enriched in antigen presentation molecules such as major histocompatibility complex I and II (MHC-I and MHC-II), tetraspanins (e.g., CD63), several heat shock proteins, cytoskeletal components such as actins and tubulins, proteins involved in intracellular membrane fusion, cell-cell interactions (e.g. CD54), signal transduction proteins, and cytosolic enzymes.

Exosomes may mediate transfer of cellular proteins from one cell (e.g., a parental cells) to a target or recipient cell by fusion of the exosomal membrane with the plasma membrane of the target cell. As such, modifying the material that is encapsulated by the exosome provides a mechanism by which exogenous agents, such as the polynucleotides described herein, may be introduced to a target cell. Exosomes that have been modified to contain one or more exogenous agents (e.g., a polynucleotide described herein) are referred to herein as “modified exosomes”. In some embodiments, modified exosomes are produced by introduction of the exogenous agent (e.g., a polynucleotides described herein) are introduced into a parental cell. In such embodiments, an exogenous nucleic acid is introduced into the parental, exosome-producing cells such that the exogenous nucleic acid itself, or a transcript of the exogenous nucleic acid is incorporated into the modified exosomes produced from the parental cell. The exogenous nucleic acids can be introduced to the parental cell by means known in the art, for example transduction, transfection, transformation, and/or microinjection of the exogenous nucleic acids.

In some embodiments, modified exosomes are produced by directly introducing a polynucleotide described herein into an exosome. In some embodiments, a polynucleotide described herein is introduced into an intact exosome. “Intact exosomes” refer to exosomes comprising proteins and/or genetic material derived from the parental cell from which they are produced. Methods for obtaining intact exosomes are known in the art (See e.g., Alvarez-Erviti L. et al., Nat Biotechnol. 2011 April; 29(4):34-5; Ohno S, et al., Mol Ther 2013 January; 21(1):185-91; and EP Patent Publication No. 2010663).

In particular embodiments, exogenous agents (e.g., the polynucleotides described herein) are introduced into empty exosomes. “Empty exosomes” refer to exosomes that lack proteins and/or genetic material (e.g., DNA or RNA) derived from the parental cell. Methods to produce empty exosomes (e.g., lacking parental cell-derived genetic material) are known in the art including UV-exposure, mutation/deletion of endogenous proteins that mediate loading of nucleic acids into exosomes, as well as electroporation and chemical treatments to open pores in the exosomal membranes such that endogenous genetic material passes out of the exosome through the open pores. In some embodiments, empty exosomes are produced by opening the exosomes by treatment with an aqueous solution having a pH from about 9 to about 14 to obtain exosomal membranes, removing intravesicular components (e.g., intravesicular proteins and/or nucleic acids), and reassembling the exosomal membranes to form empty exosomes. In some embodiments, intravesicular components (e.g., intravesicular proteins and/or nucleic acids) are removed by ultracentrifugation or density gradient ultracentrifugation. In some embodiments, the membranes are reassembled by sonication, mechanical vibration, extrusion through porous membranes, electric current, or combinations of one or more of these techniques. In particular embodiments, the membranes are reassembled by sonication.

In some embodiments, loading of intact or empty exosomes with exogenous agents (e.g., the polynucleotides described herein) to produce a modified exosome can be achieved using conventional molecular biology techniques such as in vitro transformation, transfection, and/or microinjection. In some embodiments, the exogenous agents (e.g., the polynucleotides described herein) are introduced directly into intact or empty exosomes by electroporation. In some embodiments, the exogenous agents (e.g., the polynucleotides described herein) are introduced directly into intact or empty exosomes by lipofection (e.g., transfection). Lipofection kits suitable for use in the production of exosome according to the present disclosure are known in the art and are commercially available (e.g., FuGENE® HD Transfection Reagent from Roche, and LIPOFECTAMINE™ 2000 from Invitrogen). In some embodiments, the exogenous agents (e.g., the polynucleotides described herein) are introduced directly into intact or empty exosomes by transformation using heat shock. In such embodiments, exosomes isolated from parental cells are chilled in the presence of divalent cations such as Ca²⁻ (in CaCl₂) in order to permeabilize the exosomal membrane. The exosomes can then be incubated with the exogenous nucleic acids and briefly heat shocked (e.g., incubated at 42° C. for 30-120 seconds). In particular embodiments, transformation of intact or empty exosomes using heat shock methods are used when the exogenous nucleic acid is a circular DNA plasmid. In particular embodiments, loading of empty exosomes with exogenous agents (e.g., the polynucleotides described herein) can be achieved by mixing or co-inbucation of the agents with the exosomal membranes after the removal of intravesicular components. The modified exosomes reassembled from the exosomal membranes will therefore incorporate the exogenous agents into the intravesicular space. Additional methods for producing exosome encapsulated nucleic acids are known in the art (See e.g., U.S. Pat. Nos. 9,889,210; 9,629,929; and 9,085,778; International PCT Publication Nos. WO 2017/161010 and WO 2018/039119).

Exosomes can be obtained from numerous different parental cells, including cell lines, bone-marrow derived cells, and cells derived from primary patient samples. Exosomes released from parental cells can be isolated from supernatants of parental cell cultures by means known in the art. For example, physical properties of exosomes can be employed to separate them from a medium or other source material, including separation on the basis of electrical charge (e.g., electrophoretic separation), size (e.g., filtration, molecular sieving, etc), density (e.g., regular or gradient centrifugation) and Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). Analysis of exosomal surface proteins can be determined by flow cytometry using fluorescently labeled antibodies for exosome-associated proteins such as CD63. Additional markers for characterizing exosomes are described in International PCT Publication No. WO 2017/161010. In yet further contemplated methods, the exosomes can also be fused using chemical and/or physical methods, including PEG-induced fusion and/or ultrasonic fusion.

In some embodiments, size exclusion chromatography can be utilized to isolate the exosomes. In some embodiments, the exosomes can be further isolated after chromatographic separation by centrifugation techniques (of one or more chromatography fractions), as is generally known in the art. In some embodiments, the isolation of exosomes can involve combinations of methods that include, but are not limited to, differential centrifugation as previously described (See Raposo, G. et al., J. Exp. Med. 183, 1161-1172 (1996)), ultracentrifugation, size-based membrane filtration, concentration, and/or rate zonal centrifugation.

In some embodiments, the exosomal membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans. Exemplary exosomal membrane compositions and methods for modifying the relative amount of one or more membrane component are described in International PCT Publication No. WO 2018/039119.

Preferably, the particles described herein are nanoscopic in size, in order to enhance solubility, avoid possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

In some embodiments, the particles are exosomes and have a diameter between about 30 and about 100 nm, between about 30 and about 200 nm, or between about 30 and about 500 nm. In some embodiments, the particles are exosomes and have a diameter between about 10 nm and about 100 nm, between about 20 nm and about 100 nm, between about 30 nm and about 100 nm, between about 40 nm and about 100 nm, between about 50 nm and about 100 nm, between about 60 nm and about 100 nm, between about 70 nm and about 100 nm, between about 80 nm and about 100 nm, between about 90 nm and about 100 nm, between about 100 nm and about 200 nm, between about 100 nm and about 150 nm, between about 150 nm and about 200 nm, between about 100 nm and about 250 nm, between about 250 nm and about 500 nm, or between about 10 nm and about 1000 nm. In some embodiments, the particles are exosomes and have a diameter between about 20 nm and 300 nm, between about 40 nm and 200 nm, between about 20 nm and 250 nm, between about 30 nm and 150 nm, or between about 30 nm and 100 nm.

B. Lipid Nanoparticles

In certain embodiments, the recombinant DNA molecules described herein are encapsulated in a lipid nanoparticle (LNP). In certain embodiments, the LNP comprises one or more lipids such as such as triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). In some embodiments, the LNP comprises a cationic lipid and one or more helper lipids.

Cationic lipids refer to any of a number of lipid species that carry a net positive charge at a selected pH, such as physiological pH. Such lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C₁₈ alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992 incorporated herein by reference.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle.

In some embodiments, the LNPs comprise one or more non-cationic helper lipids. Exemplary helper lipids include (1,2-dilauroyl-sn-glycero-3-phosphoethanolamine) (DLPE), 1,2-diphytanoyl-sn-glycero-3-phosphoethanolamine (D iPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-distearoyl-sn-glycero-3-phosphoethanolamine (DSPE), ceramides, sphingomyelins, and cholesterol.

The use and inclusion of polyethylene glycol (PEG)-modified phospholipids and derivatized lipids such as derivatized ceramides (PEG-CER), including N-octanoyl-sphingosine-1-[succinyl(methoxy polyethylene glycol)-2000] (C8 PEG-2000 ceramide) in the liposomal and pharmaceutical compositions described herein is also contemplated, preferably in combination with one or more of the compounds and lipids disclosed herein.

In some embodiments, the lipid nanoparticles may further comprise one or more of PEG-modified lipids that comprise a poly(ethylene)glycol chain of up to 5 kDa in length covalently attached to a lipid comprising one or more C6-C20 alkyls. In some embodiments, the LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, or about 1.0%, of the total lipid content in the lipid nanoparticle.

In some embodiments, the LNP comprises a cationic lipid and one or more helper lipids, wherein the cationic lipid is DOTAP. In some embodiments, the LNP comprises a cationic lipid and one or more helper lipids, wherein the one or more helper lipids comprises cholesterol. In some embodiments, the LNP comprises a cationic lipid and one or more helper lipids, wherein the one or more helper lipids comprises DLPE. In some embodiments, the LNP comprises a cationic lipid and one or more helper lipids, wherein the one or more helper lipids comprises DOPE. In some embodiments, the LNP comprises a cationic lipid and at least two helper lipids, wherein the cationic lipid is DOTAP, and the at least two helper lipids comprise cholesterol and DLPE. In some embodiments, the at least two helper lipids comprise cholesterol and DOPE. In some embodiments, the LNP comprises a cationic lipid and at least three helper lipids, wherein the cationic lipid is DOTAP, and the at least three helper lipids comprise cholesterol, DLPE, and DSPE. In some embodiments, the at least three helper lipids comprise cholesterol, DOPE, and DSPE. In some embodiments, the LNP comprises DOTAP, cholesterol, and DLPE. In some embodiments, the LNP comprises DOTAP, cholesterol, and DOPE. In some embodiments, the LNP comprises DOTAP, cholesterol, DLPE, and DSPE. In some embodiments, the LNP comprises DOTAP, cholesterol, DLPE, and DSPE-PEG. In some embodiments, the LNP comprises DOTAP, cholesterol, DOPE, and DSPE. In some embodiments, the LNP comprises DOTAP, cholesterol, DOPE, and DSPE-PEG.

In some embodiments, the LNP comprises DOTAP, cholesterol (Chol), and DLPE, wherein the ratio of DOTAP:Chol:DLPE (as a percentage of total lipid content) is about 50:35:15. In some embodiments, the LNP comprises DOTAP, cholesterol (Chol), and DLPE, wherein the ratio of DOTAP:Chol:DOPE (as a percentage of total lipid content) is about 50:35:15. In some embodiments, the LNP comprises DOTAP, cholesterol (Chol), DLPE, DSPE-PEG, wherein the ratio of DOTP:Chol:DLPE (as a percentage of total lipid content) is about 50:35:15 and wherein the particle comprises about 0.2% DSPE-PEG. In some embodiments, the LNP comprises an ionizable lipid, e.g., a 7.SS-cleavable and pH-responsive Lipid Like, Material (such as the COATSOME® SS-Series). Additional examples of cationic or ionizable lipids suitable for the formulations and methods of the disclosure are described in, e.g., WO2018089540A1, WO2017049245A2, US20150174261, US2014308304, US2015376115, WO201/199952, and WO2016/176330.

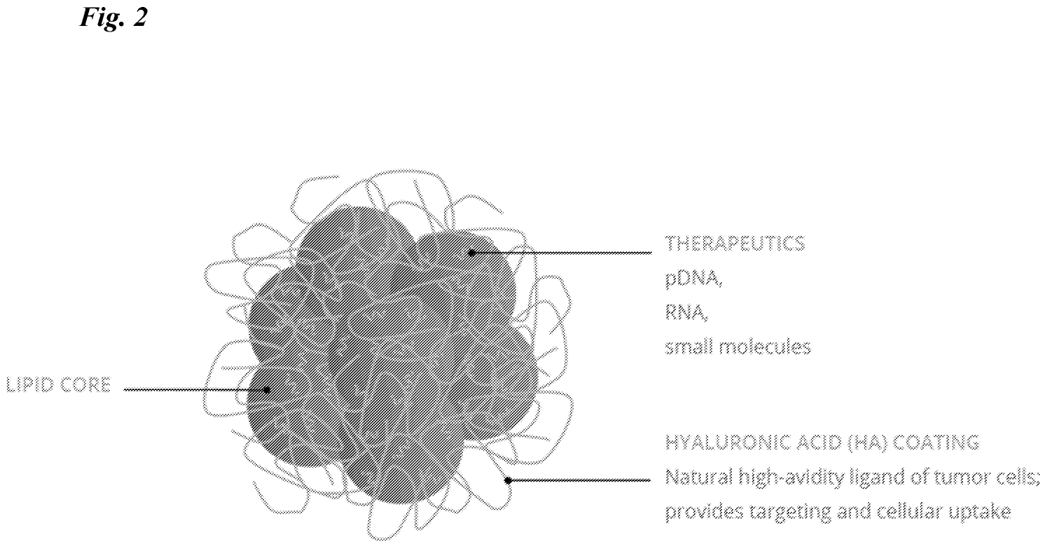

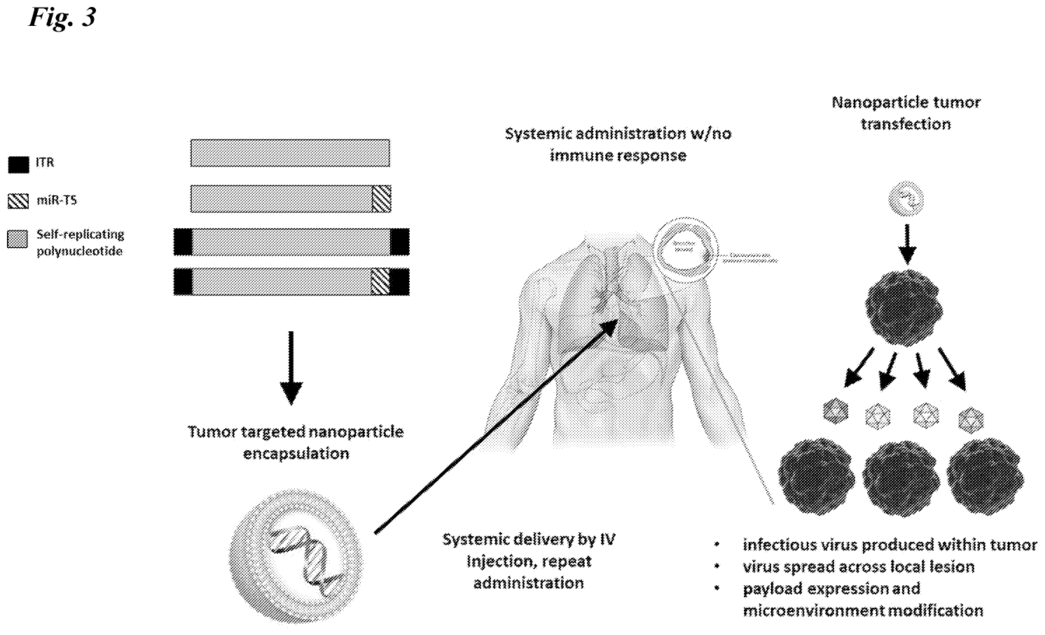

In some embodiments, the nanoparticle is coated with a glycosaminoglycan (GAG) in order to modulate or facilitate uptake of the nanoparticle by target cells (FIG. 2). The GAG may be heparin/heparin sulfate, chondroitin sulfate/dermatan sulfate, keratin sulfate, or hyaluronic acid (HA). In a particular embodiment, the surface of the nanoparticle is coated with HA and targets the particles for uptake by tumor cells. In some embodiments, the lipid nanoparticle is coated with an arginine-glycine-aspartate tri-peptide (RGD peptides) (See Ruoslahti, Advanced Materials, 24, 2012, 3747-3756; and Bellis et al., Biomaterials, 32(18), 2011, 4205-4210).

In some embodiments, the LNPs have an average size of about 150 nm to about 500 nm. For example, in some embodiments, the LNPs have an average size of about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

In some embodiments, the LNPs have an average zeta-potential of less than about −20 mV. For example in some embodiments, the LNPs have an average zeta-potential of less than about less than about −30 mV, less than about 35 mV, or less than about −40 mV. In some embodiments, the LNPs have an average zeta-potential of between about −50 mV to about −20 mV, about −40 mV to about −20 mV, or about −30 mV to about −20 mV. In some embodiments, the LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In some embodiments, the lipid nanoparticles comprise a recombinant nucleic acid molecule described herein and comprise a ratio of lipid (L) to nucleic acid (N) of about 3:1 (L:N). In some embodiments, the lipid nanoparticles comprise a recombinant nucleic acid molecule described herein and comprise an L:N ratio about 4:1, about 5:1, about 6:1, or about 7:1. In some embodiments, the lipid nanoparticles comprise a recombinant nucleic acid molecule described herein and comprise an L:N ratio about 4.5:1, about 4.6:1, about 4.7:1, about 4.8:1, about 4.9:1, about 5:1, about 5.1:1, about 5.2:1, about 5.3:1, about 5.4:1, or about 5.5:1.

### VI. Therapeutic Compositions and Methods of Use

One aspect of the disclosure relates to therapeutic compositions comprising the recombinant nucleic acid molecules described herein, or particles comprising a recombinant nucleic acid molecule described herein, and methods for the treatment of cancer. Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The present disclosure provides methods of killing a cancerous cell or a target cell comprising exposing the cell to a polynucleotide or particle described herein, or composition thereof, under conditions sufficient for the intracellular delivery of the composition to the cancerous cell. As used herein, a “cancerous cell” or a “target cell” refers to a mammalian cell selected for treatment or administration with a polynucleotide or particle described herein, or composition thereof described herein. As used herein “killing a cancerous cell” refer specifically to the death of a cancerous cell by means of apoptosis or necrosis. Killing of a cancerous cell may be determined by methods known in the art including but not limited to, tumor size measurements, cell counts, and flow cytometry for the detection of cell death markers such as Annexin V and incorporation of propidium idodide.

The present disclosure further provides for a method of treating or preventing cancer in a subject in need thereof wherein an effective amount of the therapeutic compositions described herein is administered to the subject. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. The encapsulated polynucleotide compositions described herein are particularly useful in the treatment of metastatic cancers, wherein systemic administration may be necessary to deliver the compositions to multiple organs and/or cell types. Therefore, in a particular embodiment, the compositions described herein are administered systemically.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival or improvements in the quality of life.

In some embodiments, administration of an effective dose may be achieved with administration a single dose of a composition described herein. As used herein, “dose” refers to the amount of a composition delivered at one time. In some embodiments, a dose may be measured by the number of particles in a given volume (e.g., particles/mL). In some embodiments, a dose may be further refined by the genome copy number of the polynucleotides described herein present in each particle (e.g., # of particles/mL, wherein each particle comprises at least one genome copy of the polynucleotide). In some embodiments, delivery of an effective dose may require administration of multiple doses of a composition described herein. As such, administration of an effective dose may require the administration of at least 2, 3, 4, 5, 6, 7, 8, 9, 10, 20, 30, 40, 50, or more doses of a composition described herein.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

As used herein “prevention” or “prophylaxis” can mean complete prevention of the symptoms of a disease, a delay in onset of the symptoms of a disease, or a lessening in the severity of subsequently developed disease symptoms.

The term “subject” or “patient” as used herein, is taken to mean any mammalian subject to which a composition described herein is administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish, and reptiles.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endothel io sarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein.

A. Exemplary Self-Replicating Polynucleotides

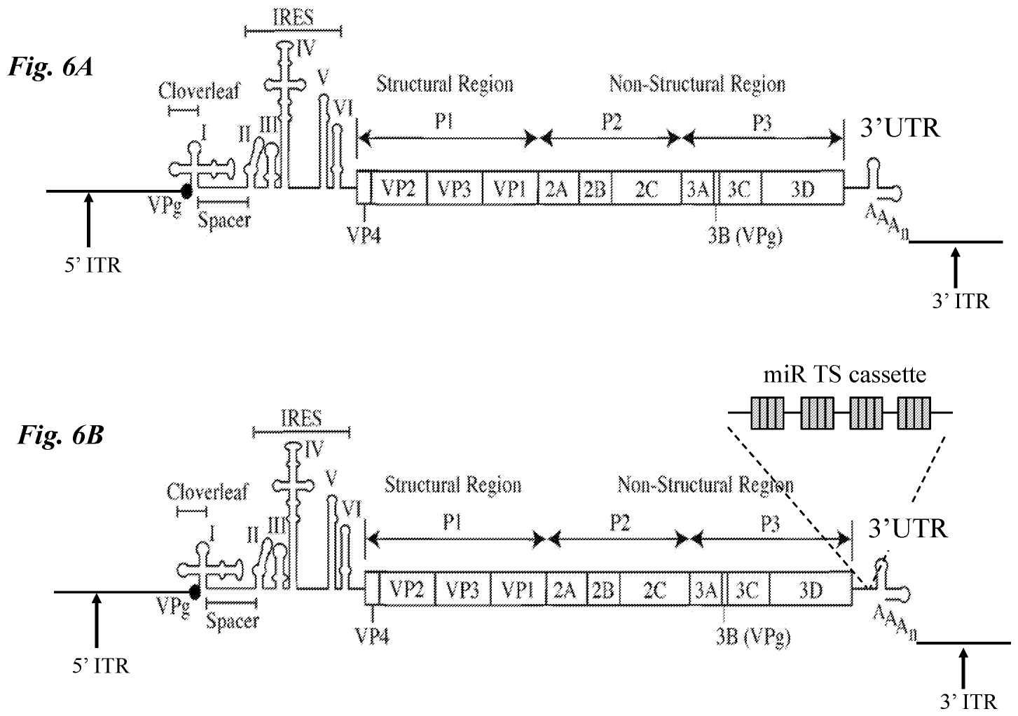

One of skill in the art will understand that the nature of the encoded virus will vary and will depend on the disease indication to be treated. For example, in some embodiments, a polio virus may be used in the treatment of a particular cancer. The polio virus genome comprises a single-stranded, positive-sense polarity RNA molecule which encodes a single polyprotein. The 5′ un-translated region (UTR) harbors two functional domains, the cloverleaf and the internal ribosome entry site (IRES), and is covalently linked to the viral protein, VPg. The 3′UTR is poly-adenylated (See e.g., FIG. 6A). In some embodiments, the polio virus genome is flanked on the 5′ and 3′ ends by AAV-derived ITRs (See e.g., FIG. 6A).

In some embodiments, one or more miRNA target sequences are operatively linked to a viral gene, e.g. an essential viral gene. For example, the polio virus genome comprises several genes suitable for this purpose, including without limitation: 3D^{pol}, an RNA dependent RNA polymerase whose function is to make multiple copies of the viral RNA genome; 2A^{pro} and 3C^{pro}/3CD^{pro}, proteases which cleave the viral polypeptide VPg (3B), a protein that binds viral RNA and is necessary for synthesis of viral positive and negative strand RNA; 2BC, 2B, 2C (an ATPase), 3AB, 3A, 3B proteins which comprise the protein complex needed for virus replication; VP0, which is further cleaved into VP2 and VP4, VP1 and VP3, proteins of the viral capsid. In some embodiments, the miRNA-attenuated polio virus genome is flanked by AAV-derived ITR sequences to aid in polynucleotide replication and nuclear entry (See e.g., FIG. 6B). Other genes may be selected as appropriate. In some embodiments, miRNA target sequences are operatively linked to a viral gene, e.g., an essential viral gene, by insertion of the miRNA target sequence in a location within the gene locus that results in transcription of the miRNA target sequence while maintaining the ability of the gene to code for a functional polypeptide. In some embodiments, the miRNA target sequence is inserted into the 5′ UTR or the 3′ UTR of the viral gene. In some embodiments, the miRNA target sequence is inserted into the open reading frame, such as, for example, between the coding sequences of two polypeptides such that the miRNA target sequence is in-frame permitting translation and post-translational cleavage of the polypeptide into two or more functional proteins. For example, the miRNA target sequence can be inserted between two 2A peptide sequences and additional nucleotides added as necessary to preserve the reading frame of polypeptide sequence downstream (3′) to the insertion site of the miRNA target sequence.

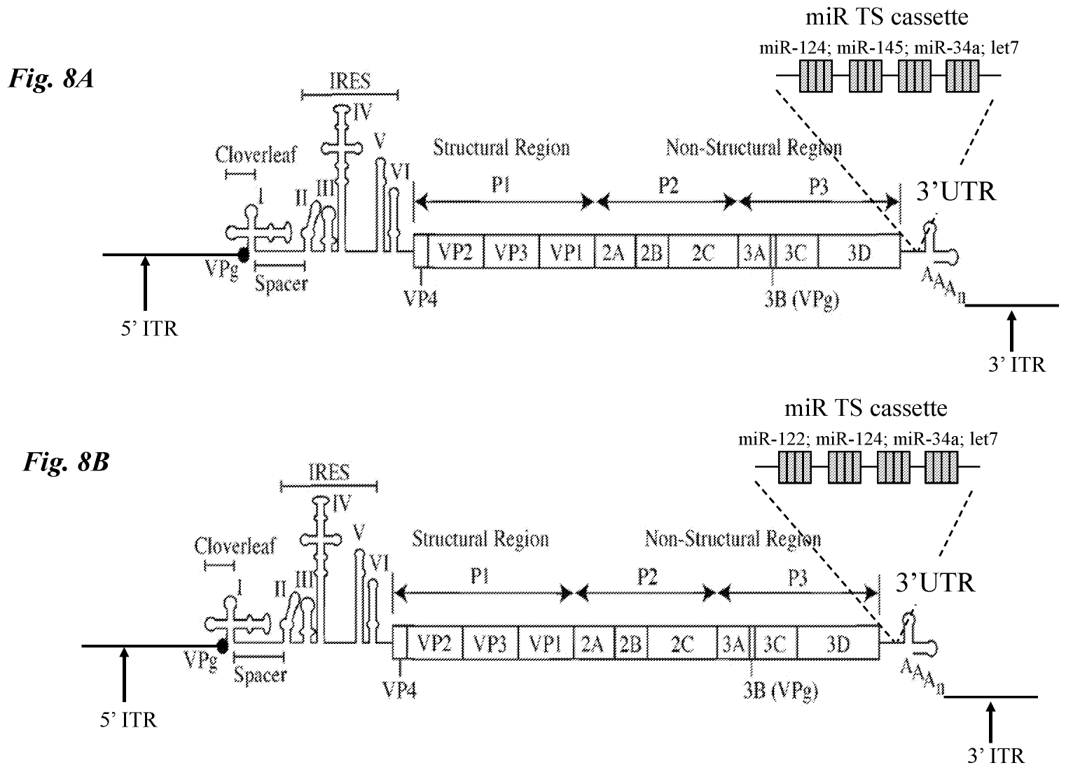

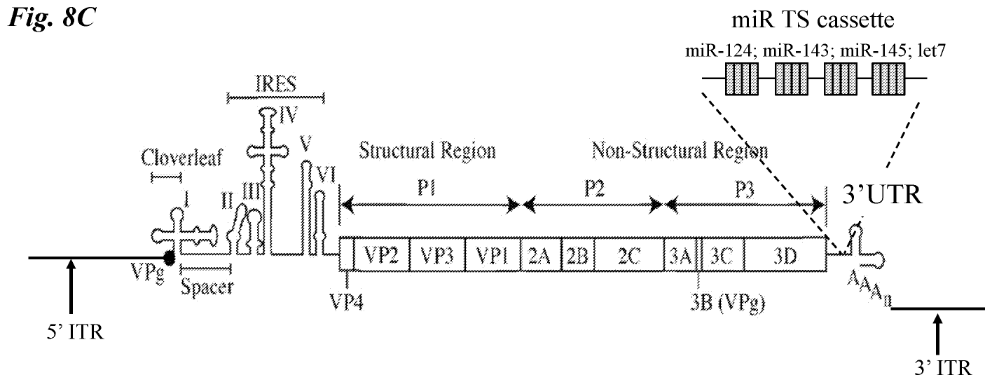

In some embodiments, the wild-type polio virus genome is modified by insertion of a miRNA target sequence cassette containing tetrameric miR-124, miR-145, miR-34a, and let7 target sites into the 3′ UTR for attenuation of one or more essential polio viral genes (FIG. 8A). In some embodiments, this miRNA-attenuated polio virus is suitable for use in the treatment of non-small cell lung cancer (FIG. 8A). In some embodiments, the wild-type PV genome is modified by insertion of a miRNA target sequence cassette containing tetrameric miR-122, miR-124, miR-34a, and let7 target sites into the 3′ UTR of one or more essential polio viral genes (FIG. 8B). In some embodiments, this miRNA-attenuated polio virus is suitable for use in the treatment of hepatocellular carcinoma (FIG. 8B). In some embodiments, the wild-type polio virus genome is modified by insertion of a miRNA target sequence cassette containing tetrameric miR-124, miR-143, miR-145, and let7 target sites into the 3′ UTR for attenuation of one or more essential polio viral genes (FIG. 8C). In some embodiments, this miRNA-attenuated polio virus is suitable for use in the treatment of prostate cancer (FIG. 8C).

In some embodiments, a VSV may be used in the treatment of a particular cancer. The VSV genome comprises a single-stranded, negative-sense polarity RNA molecule that encodes five major proteins: nucleoprotein (N), phosphoprotein (P), matrix protein (M), glycoprotein (G), and polymerase (L). There is one monocistronic mRNA for each of the five virally coded proteins. The mRNAs are capped, methylated, and polyadenylated. Since VSV is a cytoplasmic, negative-sense RNA virus, the enzymes for mRNA synthesis and modification are packaged in the virion (FIG. 9A). In some embodiments, the VSV genome is flanked by AAV-derived ITR sequences to aid in polynucleotide replication and nuclear entry (FIG. 9A).

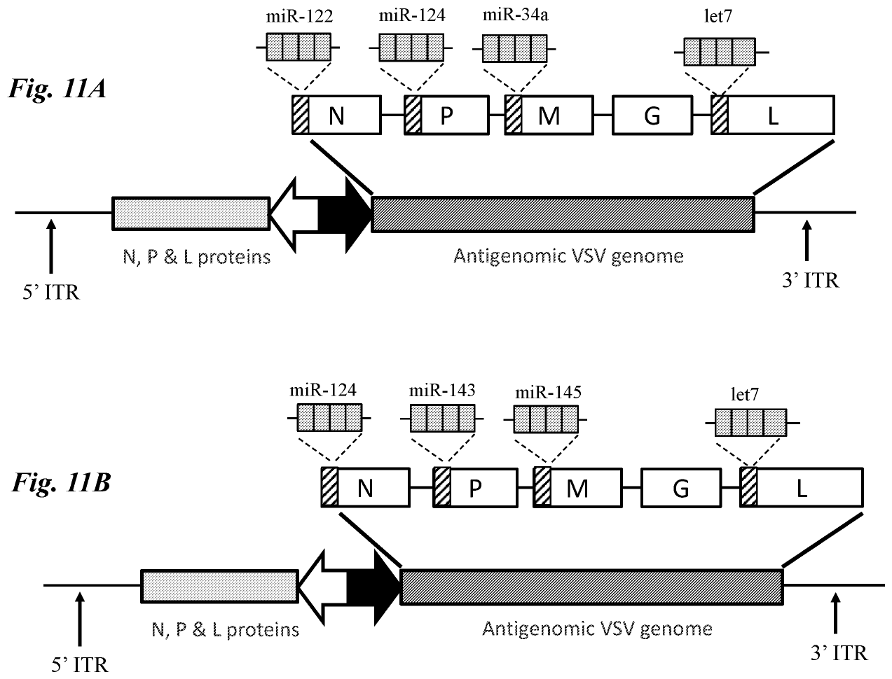

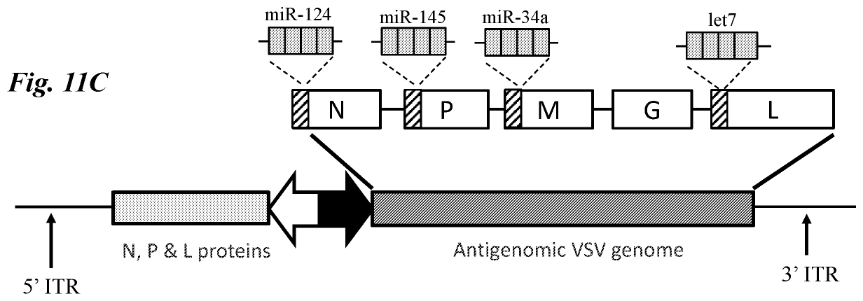

In some embodiments, the wild-type VSV genome is modified by insertion of a miRNA target sequence cassette comprising one or more miRNA target sequences inserted in the gene locus for one or more essential viral genes of the VSV genome (e.g., one or more of N, P, M, G, or L genes) (FIG. 9B). In some embodiments, the miRNA target sequence is inserted into the 5′ UTR or 3′ UTR of the gene. In some embodiments, the wild-type VSV genome is modified by insertion of a miRNA target sequence cassette comprising tetrameric miR-122, miR-124, miR-34a, and let7 target sites into the 3′ UTR of four of the five virally coded transcripts for attenuation (e.g., four of N, P, M, G, or L genes) (FIG. 11A). In some embodiments, this miRNA-attenuated VSV is suitable for use in the treatment of hepatocellular carcinoma (FIG. 11A). In some embodiments, the wild-type VSV genome is modified by insertion of a miRNA target sequence cassette comprising tetrameric miR-124, miR-143, miR-145, and let7 target sites into the 3′ UTR of four of the five virally coded transcripts for attenuation (e.g., four of N, P, M, G, or L genes) (FIG. 11B). In some embodiments, this miRNA-attenuated VSV is suitable for use in the treatment of prostate cancer (FIG. 11B). In some embodiments, the wild-type VSV genome is modified by insertion of a miRNA target sequence cassette comprising tetrameric miR-124, miR-145, miR-34a, and let7 target sites into the 3′ UTR of four of the five virally coded transcripts for attenuation (e.g., four of N, P, M, G, or L genes) (FIG. 11C). In some embodiments, this miRNA-attenuated VSV is suitable for use in the treatment of non-small cell lung cancer (FIG. 11C).

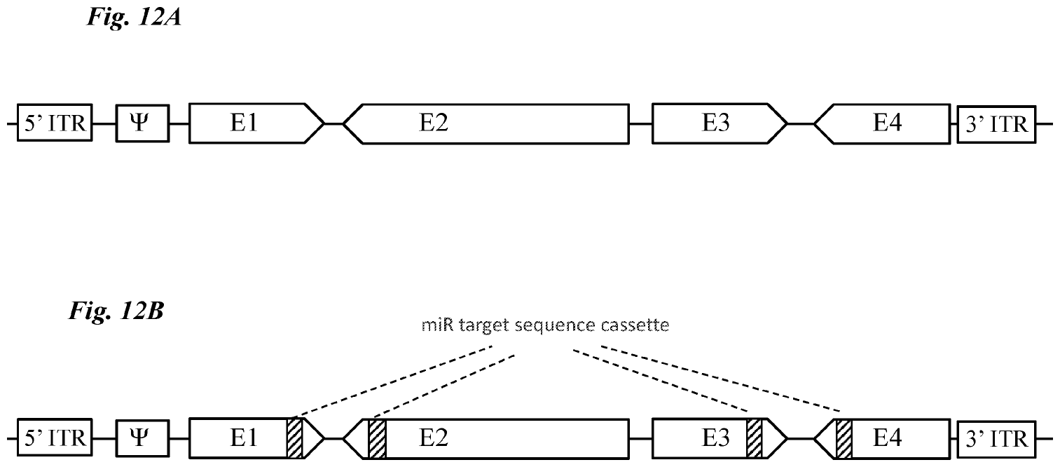

In some embodiments, an adenovirus may be used in the treatment of a particular cancer. The AAV genome comprises a double-stranded DNA molecule that encodes 24-36 protein coding genes. The E1A, E1B, E2A, E2B, E3, and E4 transcription units are transcribed early in the viral reproductive cycle (FIG. 12A). The proteins coded for by genes within these transcription units are primarily involved in regulation of viral transcription, in replication of viral DNA, and in suppression of the host response to infection. In some embodiments, the adenovirus genome is flanked by AAV-derived ITR sequences to aid in polynucleotide replication and nuclear entry (FIG. 12A).

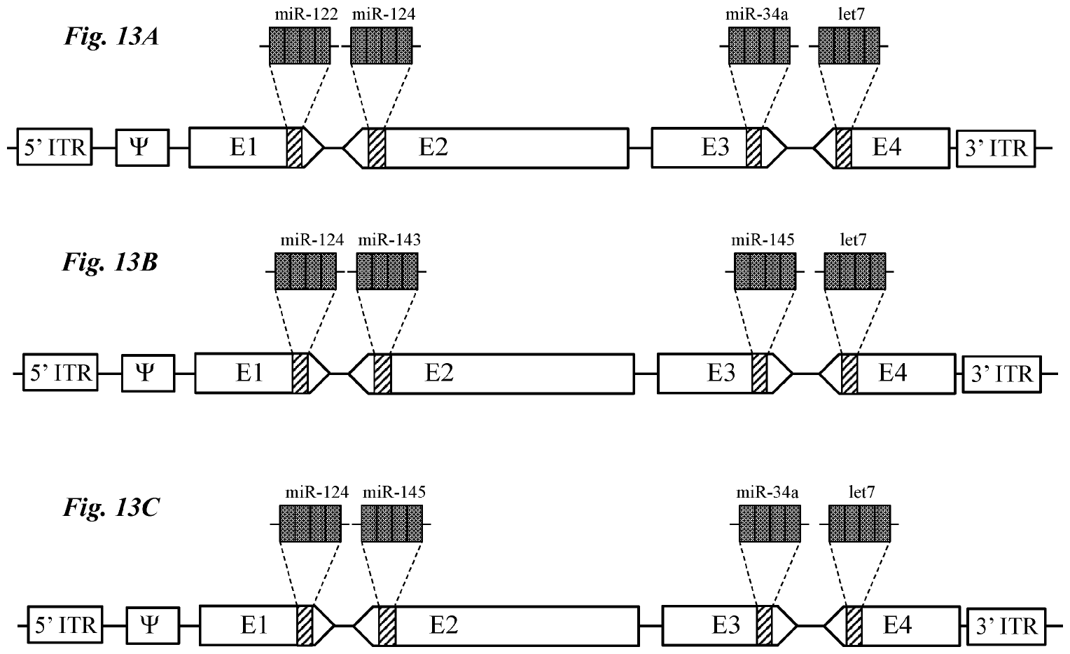

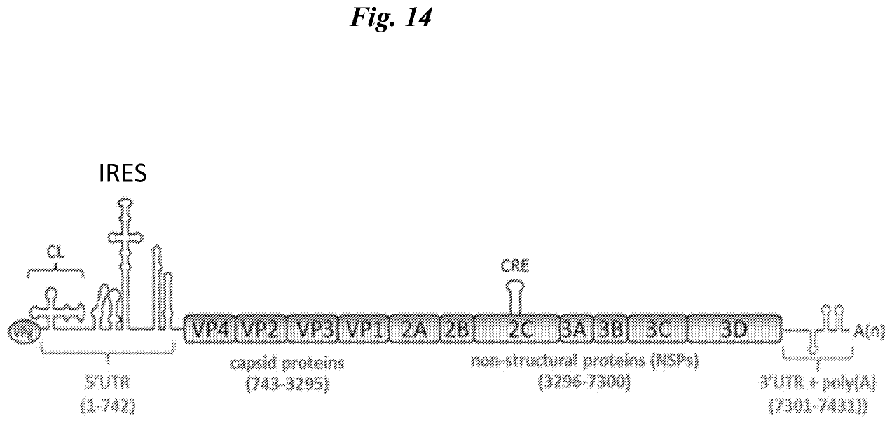

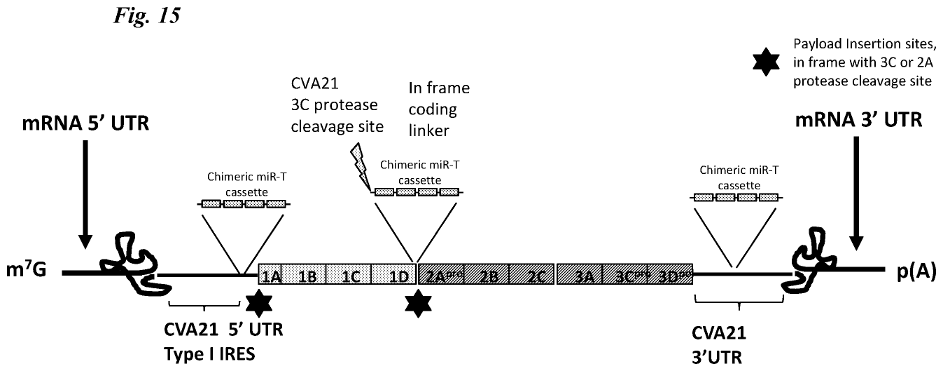

In some embodiments, the wild-type AAV genome is modified by insertion of a miRNA target sequence cassette comprising one or more miRNA target sequences inserted into one or more essential viral genes of the AAV genome (e.g., one or more of E1A, E1B, E2A, E2B, E3, or E4) (FIG. 12B). In some embodiments, the wild-type AAV genome is modified by insertion of a miRNA target sequence cassette comprising tetrameric miR-122, miR-124, miR-34a, and let7 target sites into the 3′ UTR of one or more essential genes (e.g., one or more of E1A, E1B, E2A, E2B, E3, or E4) (FIG. 13A). In some embodiments, this miRNA-attenuated adenovirus is suitable for use in the treatment of hepatocellular carcinoma (FIG. 13A). In some embodiments, the wild-type AAV genome is modified by insertion of a miRNA target sequence cassette comprising tetrameric miR-124, miR-143, miR-145, and let7 target sites into the 3′ UTR of one or more essential genes (e.g., one or more of E1A, E1B, E2A, E2B, E3, or E4) (FIG. 13B). In some embodiments, this miRNA-attenuated adenovirus is suitable for use in the treatment of prostate cancer (FIG. 13B). In some embodiments, the wild-type AAV genome is modified by insertion of a miRNA target sequence cassette comprising tetrameric miR-124, miR-145, miR-34a, and let7 target sites into the 3′ UTR of one or more essential genes (e.g., one or more of E1A, E1B, E2A, E2B, E3, or E4) (FIG. 13C). In some embodiments, this miRNA-attenuated adenovirus is suitable for use in the treatment of non-small cell lung cancer (FIG. 13C).

### EXAMPLES

The following examples are given for the purpose of illustrating various embodiments of the disclosure and are not meant to limit the present disclosure in any fashion. The present examples; along with the methods described herein are presently representative of preferred embodiments; are exemplary; and are not intended as limitations on the scope of the disclosure. Changes therein and other uses which are encompassed within the spirit of the disclosure as defined by the scope of the claims will occur to those skilled in the art.

### Example 1

### Engineering of Polynucleotide Constructs Encoding Replication-Competent Viral Genomes

The self-replicating polynucleotide constructs described herein are engineered and produced using standard molecular biology and genetics techniques. Exemplary constructs encoding particular viruses and the corresponding cancers for treatment with these constructs are described below in Tables 13, 14, and 15. However, the appropriate virus can be selected based on the desired characteristics of the virus and characteristics of the cancer to be treated. Similarly, miRNA target sequence cassettes (miR TS) can be inserted at one or more location in the viral genome to control replication of the encoded viral genome in normal, non-cancerous cells while permitting replication in cancerous cells. Exemplary constructs are described throughout the present disclosure. Constructs that have been made are summarized in Table 8 below.

TABLE 8
Polynucleotide constructs encoding
replication-competent viral genomes

| | | miR TS | | Payload | 3′ and 5′ |
|---|---|---|---|---|---|
| | miR | insertion | | insertion | genome |
| Virus | TS | location | Payload | location | modifications |
| SVV | NA | NA | NA | NA | NA |
| SVV | NA | NA | NA | NA | 5′ Hammerhead |
| | | | | | ribozyme; 3′ |
| | | | | | Hepatitis delta |
| | | | | | virus ribozyme |
| SVV | miR-1 | In-frame | NA | NA | 5′ Hammerhead |
| | and | between 2A | | | ribozyme; 3′ |
| | miR- | and 2B | | | Hepatitis delta |
| | 122 | | | | virus ribozyme |
| SVV | NA | NA | CXCL10 | In-frame | 5′ Hammerhead |
| | | | | between 2A | ribozyme; 3′ |
| | | | | and 2B | Hepatitis delta |
| | | | | | virus ribozyme |
| SVV | NA | NA | Nano- | In-frame | 5′ Hammerhead |
| | | | luc | between 2A | ribozyme; 3′ |
| | | | | and 2B | Hepatitis delta |
| | | | | | virus ribozyme |
| SVV | NA | NA | mCherry | In-frame | 5′ Hammerhead |
| | | | | between 2A | ribozyme; 3′ |
| | | | | and 2B | Hepatitis delta |
| | | | | | virus ribozyme |

After design of the self-replicating polynucleotides, the constructs are engineered for delivery by insertion into a plasmid backbone or by addition of terminal inverted repeats (ITRs) derived from an adeno-associated virus (AAV). Protocols and methods were developed for the design of these two particular types of delivery mechanisms, namely plasmid genome constructs and ITR-flanked Nano Virus (NanoV) constructs, and are described below.

### Example 2

### Design and Production of Plasmids Comprising Polynucleotide Constructs Encoding Replication-Competent Viral Genomes

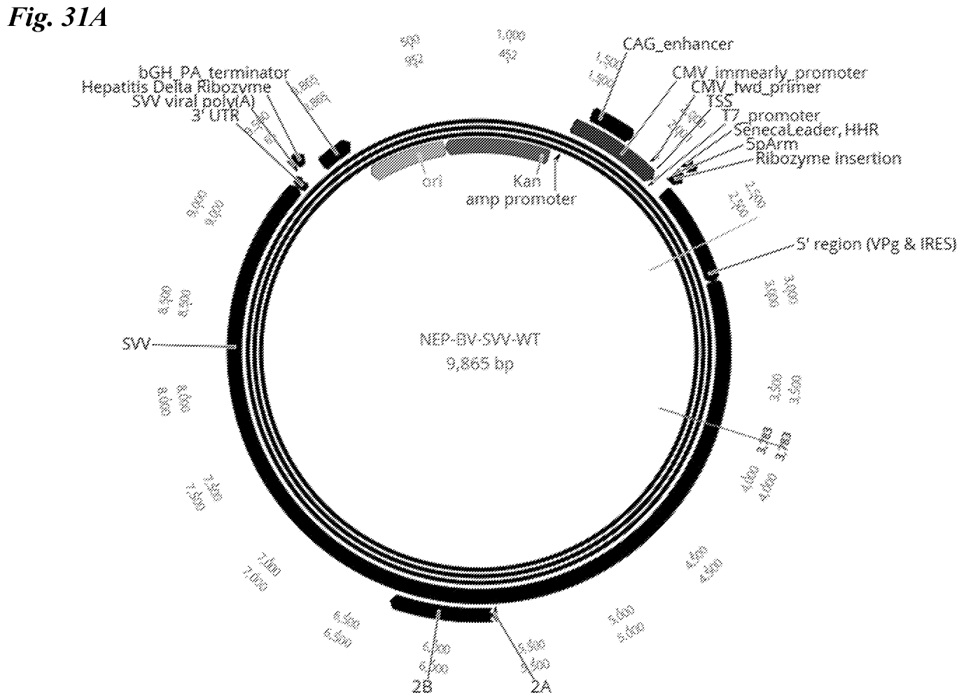

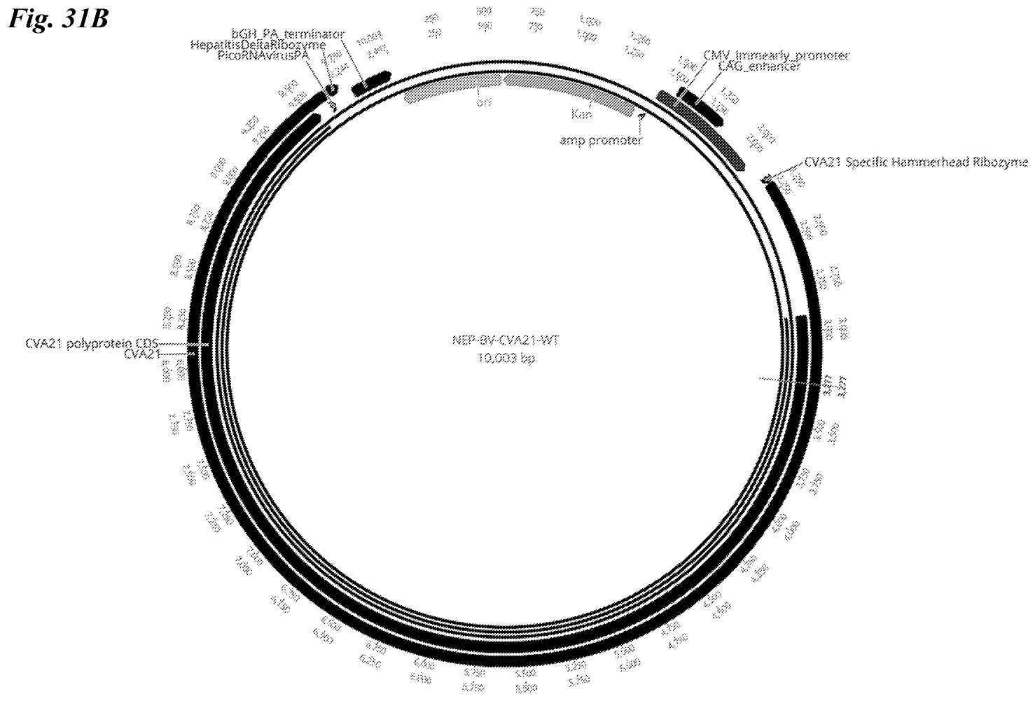

The SVV viral DNA was synthesized at Genscript, and the poly (A), the 5′ hammerhead ribozyme, and the 3′ hepatitis delta ribozyme were added with fusion PCR upon insertion with Gibson assembly into the base vector. This base vector is 2.4 kb in length and contains a minimal origin of replication and a kanamycin resistance cassette that has been optimized for use in mammalian cells (FIG. 31A). The expression cassette is disclosed as SEQ ID: 1. An analogous vector was constructed for Coxsackievirus (CVA21) and is shown in FIG. 31B. The CVA21 expression cassette is disclosed as SEQ ID NO: 2.

### Example 3

### Design and Production of ITR-Flanked NanoV Constructs

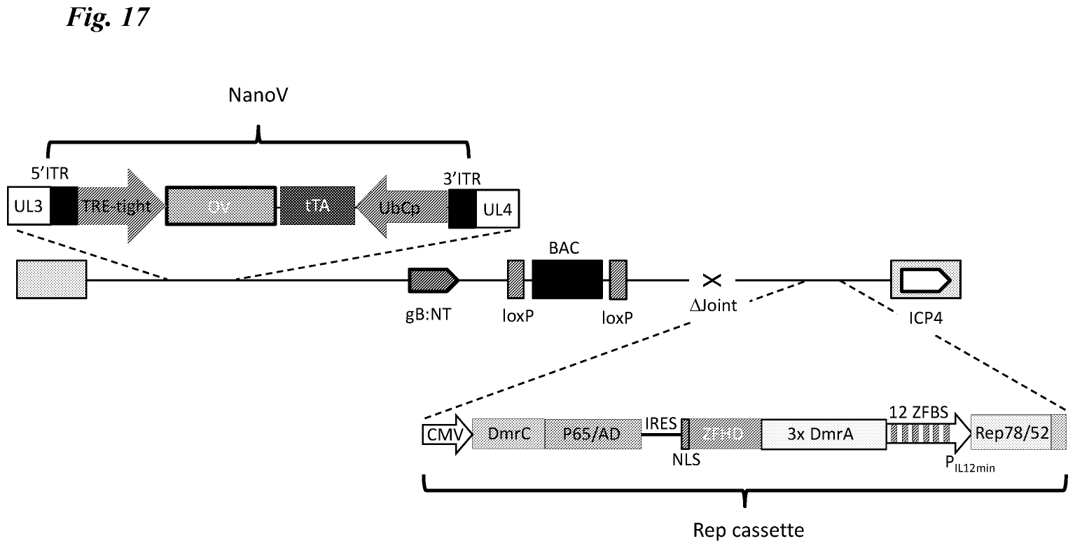

For production of ITR-flanked NanoV constructs, self-replicating polynucleotide constructs are inserted into an expression cassette flanked by AAV-derived ITRs under the control of a tetracycline (Tet) responsive promoter. FIG. 17 provides a schematic of a model NanoV construct. The tetracycline responsive promoter, TRE-tight, drives expression of mCherry, which is used as a placeholder and can be replaced with the appropriate viral genome construct (Shown as OV in FIG. 17). Expression of the tetracycline-controlled transactivator (tTA) is controlled by a constitutive promoter, shown in FIG. 17 as UbCP. This NanoV construct is inserted in the UL3/4 intergenic region of HSV-1 using the Gateway cloning system (Thermo Fisher), which allows for rapid insertion of different NanoV cassettes. Addition of tetracycline to the culture media results in Tet binding to tTA, preventing expression of the mCherry construct. Removal of Tet from the culture media therefore allows for inducible mCherry expression. Additionally, an iDimerize cassette (Takara) under the control of a second constitutive promoter (e.g., CMV) is inserted into the UL50/51 intergenic locus within the HSV-1 BAC. The iDimerize cassette comprises two heterologous dimerization domains (DmrA and DmrC) regulating heterodimerizer-inducible Rep78/52 expression. Addition of the A/C heterodimerizer AP21967 to the culture media activates the iDimerize cassette and results in Rep78/52 expression, which drives replication of ITR-flanked NanoV construct.

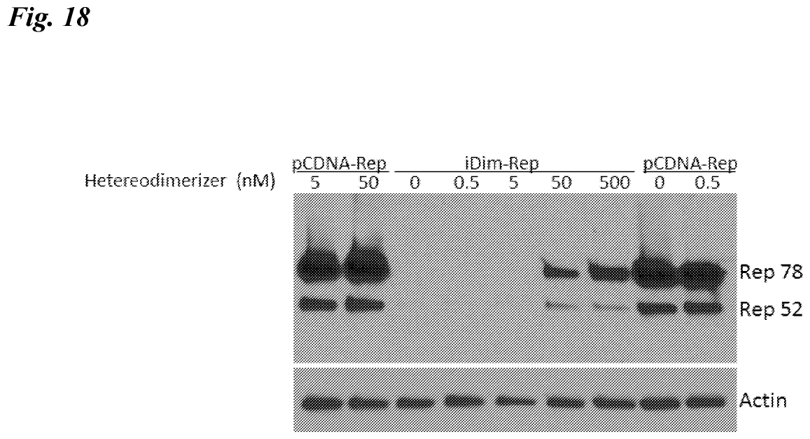

To demonstrate regulation of Rep 78/52 expression by the iDimerize cassette, Vero cells were transfected with an iDimerize-Rep cassette in the presence of AP21967 at 0.5 nm, 5 nm, 50 nm, or 500 nm. A plasmid encoding the Rep proteins (pCDNA-Rep) was used as a positive control. Protein was extracted from cells 24 hours post transfection and subjected to SDS-PAGE/Western blot analysis using α-Rep or α-Actin antibodies. As shown in FIG. 18, heterodimerizer concentrations of ≥50 nM induced Rep78/52 expression from the iDimerize cassette, while addition of the heterodimerizer had no impact on Rep expression levels in pCDNA-Rep transfected cells.

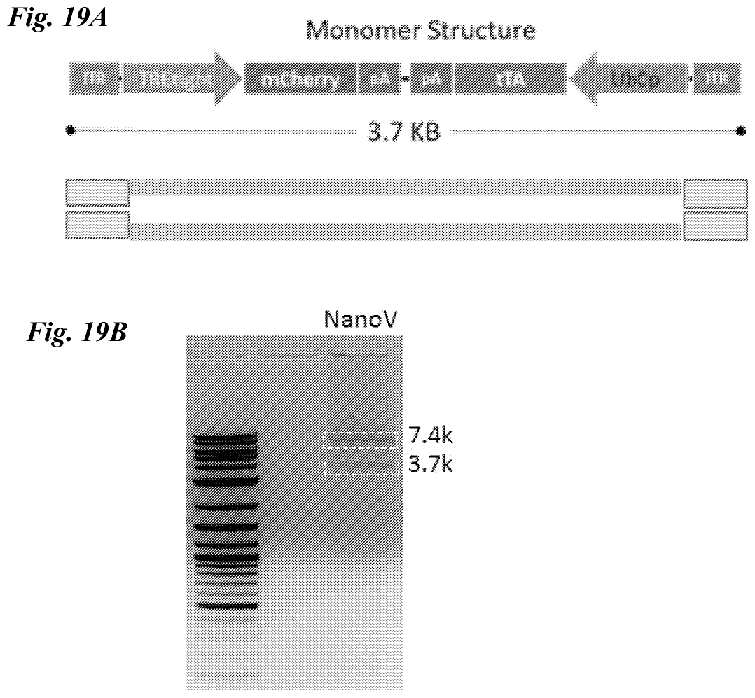

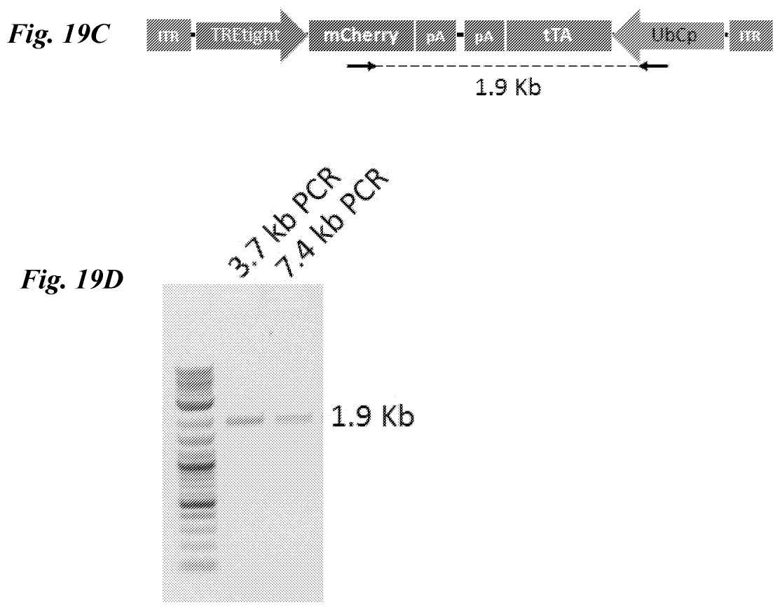

To demonstrate the production of NanoV constructs, U2OS cells were infected with the recombinant HSV-1 vectors shown in FIG. 17. After 3 days post-infection, infected cells were harvested and DNA was purified using a Miniprep DNA purification kit (Qiagen). The expected NanoV monomers and dimers produced by this system are shown in FIG. 19A. Extracted DNA was subjected to NheI and ExoIII digestion in order to expose free ends of HSV DNA, but not NanoV DNA, and degrade DNA which does not have closed ends. Digested DNA fragments were then analyzed on an agarose gel to determine the presence of the NanoV monomers and dimers. As shown in FIG. 19B, bands appear at the expected sizes for both the monomer and dimer fragments (3.7 kb and 7.4 kb, respectively). DNA was extracted from both the 3.7 kb and 7.4 kb bands and subsequent PCR analyses using internal specific for the internal mCherry cassette were performed (See schematic in FIG. 19C). As shown in FIG. 19D, these PCR reactions produced a 1.9 kb amplicon from DNA extracted from both the 3.7 and 7.4 kb bands, demonstrating that the polynucleotide sequences internal to the ITRs was replicated.

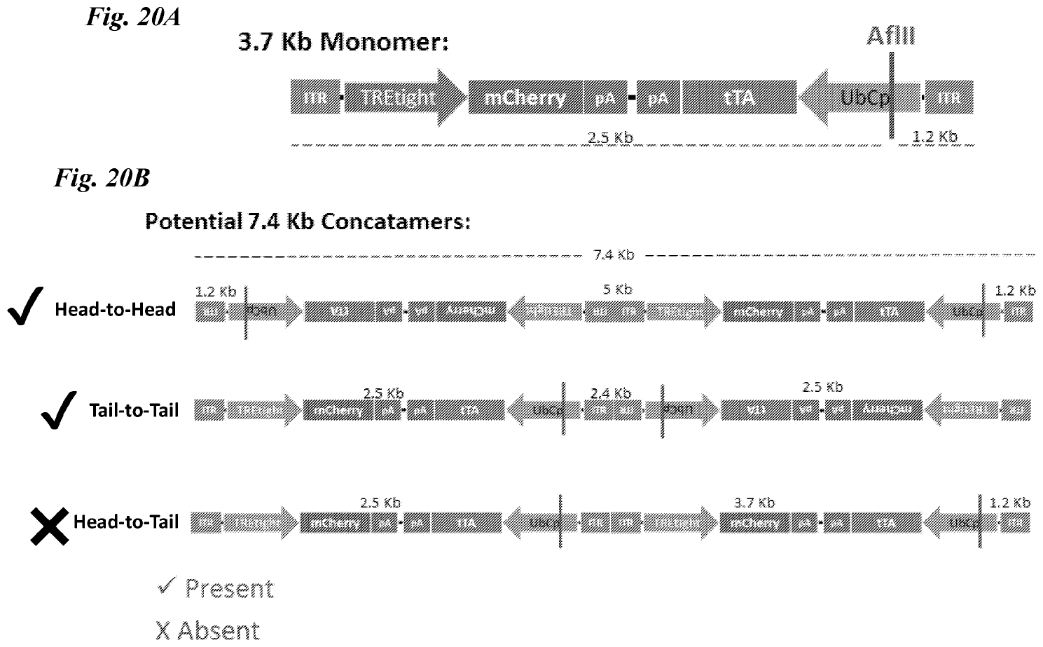

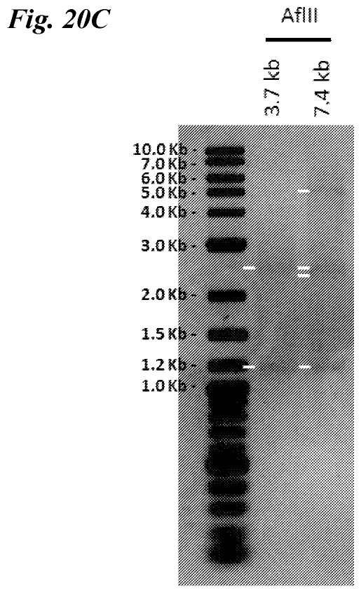

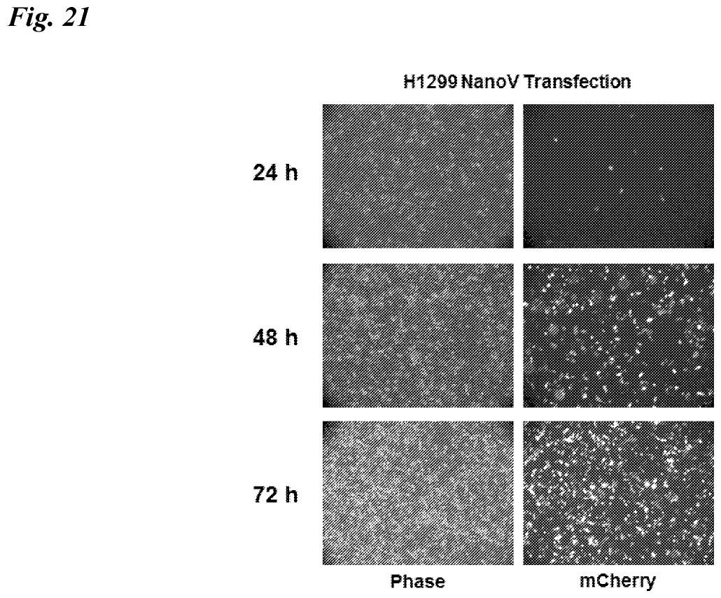

In order to determine the orientation of NanoV concatamers, DNA extracted from both 3.7 kb monomer and 7.4 kb dimers was digested with AflII and analyzed by non-reducing agarose gel electrophoresis. The expected cut site of AflII is in the UbC promoter, thereby generating cleavage products with expected sizes of 1.2 kb and 2.5 kb in the monomer, as shown in FIG. 20A. The expected product sizes from the concantamers will vary depending on the orientation of the dimers (e.g., head-to-head, tail-to-tail, or head-to-tail, as shown in FIG. 20B). AflII cleavage of DNA extracted from the 3.7 kb fragment from FIG. 18B generated the expected 1.2 kb and 2.5 kb fragments (FIG. 20C, presence of bands indicated by white bars). AflII cleavage of DNA extracted from the 7.4 kb fragment from FIG. 19B generated fragment sizes of 1.2 kb and 5 kb, indicative of tail-to-tail orientation of the concantamers, and 2.5 kb and 2.4 kb, indicative of head-to-head orientation of the concantamers.

### Example 4

### Production of Infectious Picornavirus Virus from Plasmid Genomes Requires 3′ and 5′ Ribozymes

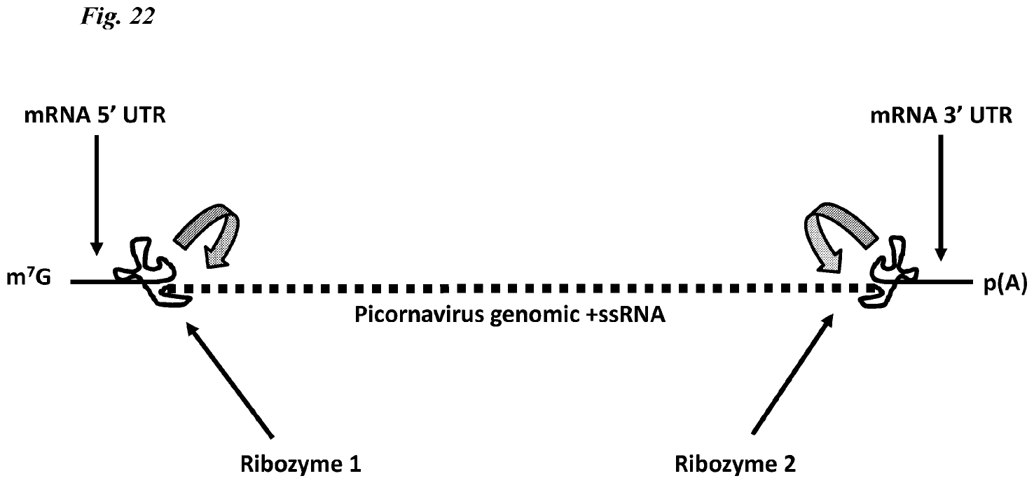

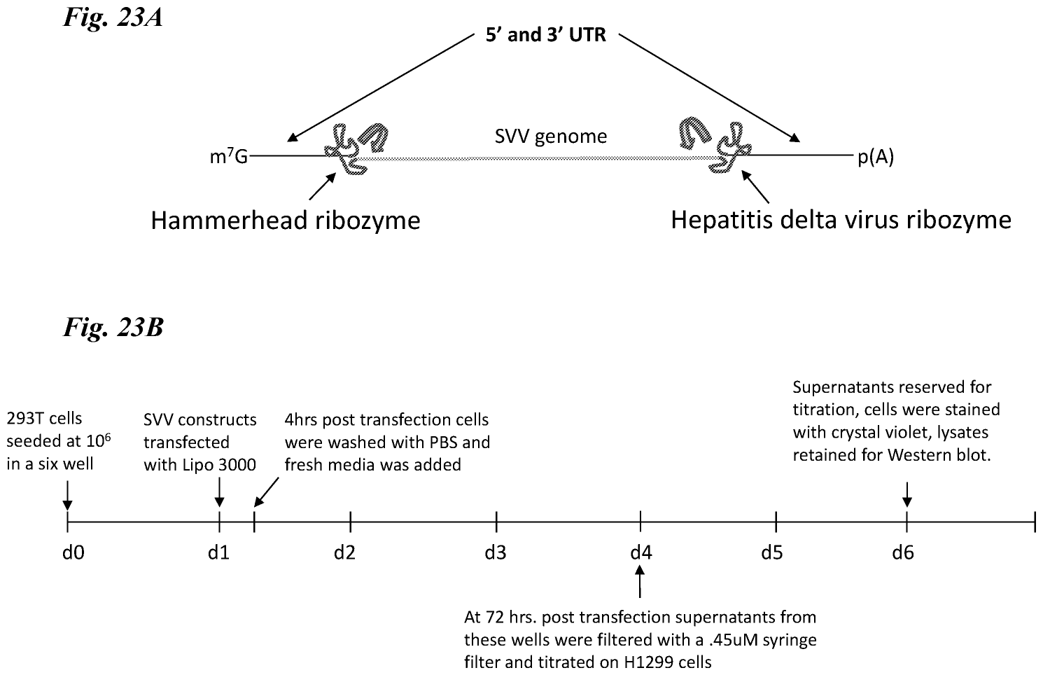

Experiments were performed to assess the ability to produce infectious SVV virus from the plasmids generated in Example 2, comprising the SVV-encoding polynucleotide under the control of a mammalian Pol II promoter. Positive-sense single stranded RNA viruses, such as SVV and Coxsackievirus, require the discrete 5′ and 3′ ends native to the virus in order to replicate properly, which are not produced by mammalian RNA Pol II transcript that contains mammalian 5′ and 3′ UTRs. Therefore, production of infectious +sense ssRNA viruses required inclusion of 5′ and 3′ ribozyme sequences which catalyzed the removal of non-viral RNA from the Pol II-encoded SVV transcript and enabled expression of replication-competent and infectious SVV (See general schematic in FIGS. 22 and 23A).

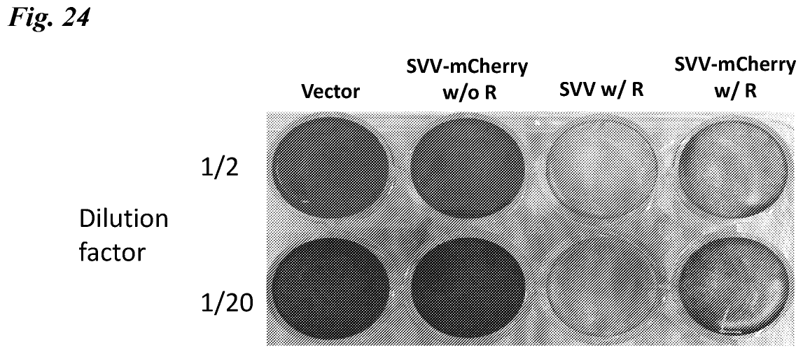

Briefly, DNA polynucleotides encoding SVV viral genomes were generated with (SVV w/ R) and without (SVV w/o R) the insertion of 5′ and 3′ ribozyme-encoding sequences (FIG. 23A). These constructs were inserted into DNA plasmids as described in Example 2. To test the ability of the SVV-encoding plasmids with and without terminal ribozyme sequences to produce infectious virus, 293T cells were seeded in 6-well plates at 1×10⁶ cells/well. 24 hours after seeding, the 293T cells were transfected with 1 ng of the SVV plasmids constructs described above in Lipofectamine 3000 for 4 hours, at which point complete media was added to each well. Supernatants from transfected 293T were collected after 72 hours, and syringe filtered with 0.45 μM filter and serially diluted onto H1299 cells (See protocol schematic in FIG. 23B). After 48 hours, supernatants were removed from the H1299 cultures and cells were stained with crystal violet to assess viral infectivity. As shown in FIG. 24, active lytic SVV was only produced from constructs comprising the terminal ribozymes, indicated by a reduced opacity in the crystal violet staining. Therefore, these data indicate that incorporation of the ribozyme-encoding sequences into the polynucleotides described herein is necessary for production of infectious SVV virus.

### Example 5

### DNA Plasmids Comprising SVV-Encoding Polynucleotide are Capable of Expressing Payload Proteins In Vitro

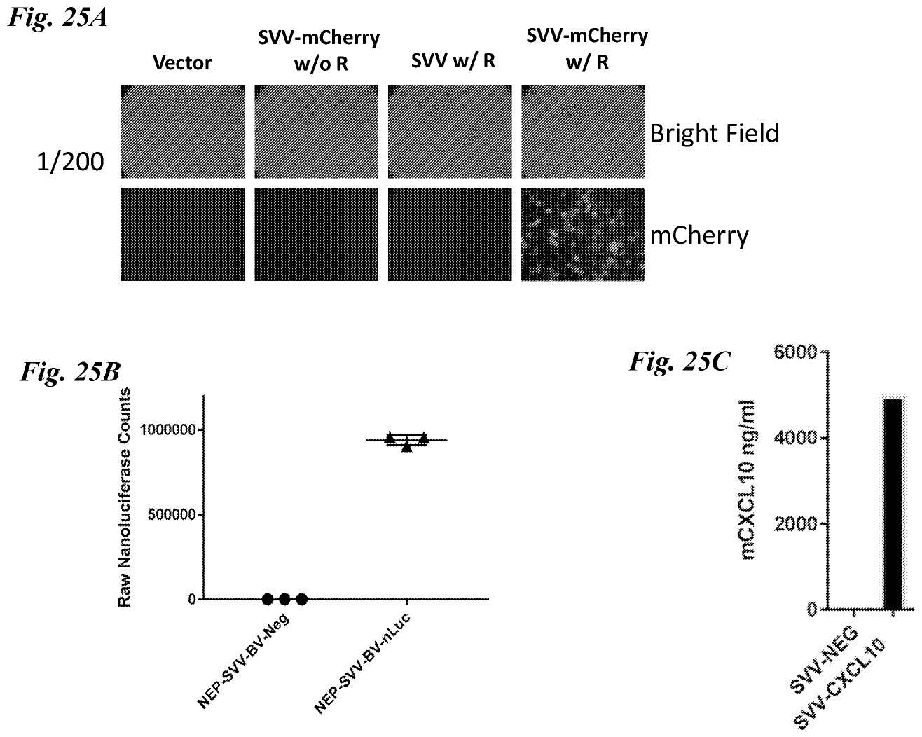

Experiments were performed to assess the ability of the SVV plasmids described in Example 2 to express payload proteins from payload-encoding sequences incorporated into the SVV-encoding polynucleotides. Three payloads were tested: an mCherry reporter, a Nanoluciferase protein, and CXCL10. SVV-encoding plasmids comprising terminal ribozyme sequences were able to express the mCherry protein, while SVV-encoding plasmids without the terminal ribozyme sequences were not (FIG. 25A). Further, the SVV-encoding plasmids were able to express Nanoluciferase (FIG. 25B). Further still, the SVV-encoding plasmids were able to express CXCL10 (FIG. 25C). These data demonstrate that, in addition to producing infectious SVV, these plasmid constructs were also able to express multiple different types of payload proteins including fluorescent proteins (exemplified by mCherry), enzymatic proteins (exemplified by Nanoluciferase), and recombinant chemokines (exemplified by CXCL10).

### Example 6

### MiRNA Attenuation of Self-Replicating Polynucleotides Encoding SVV

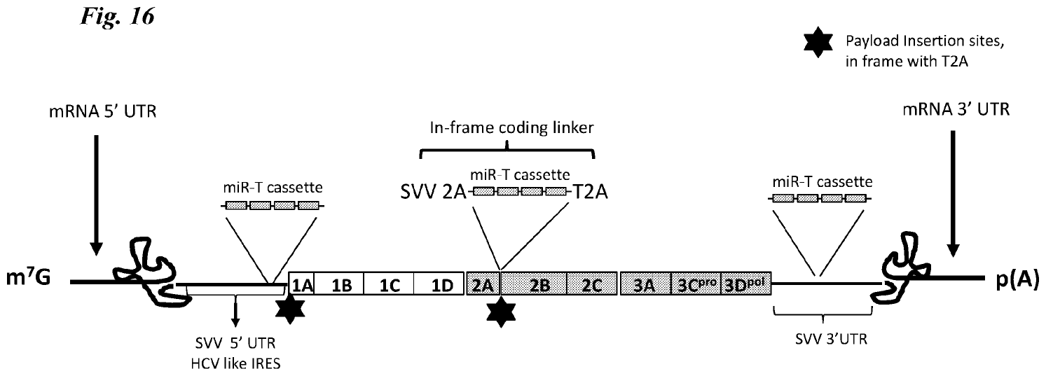

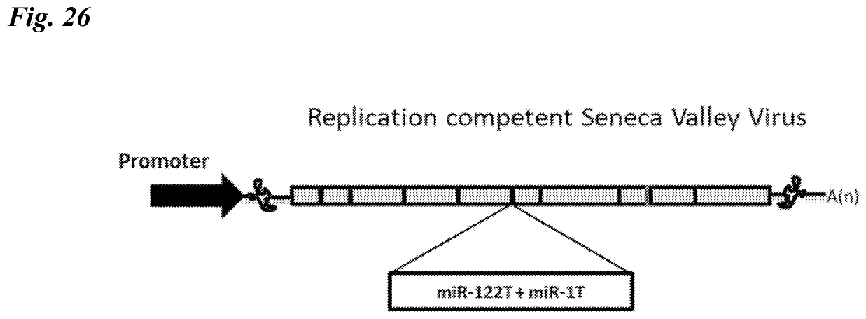

Experiments were performed to determine whether the SVV-encoding polynucleotides described in Example 2 could be miRNA attenuated. A miRNA target cassette (miR-T) with miR-1 and miR-122 target sequences were inserted in frame with the SVV viral polyprotein between the endogenous viral 2A and a synthetic T2A sequence as shown in FIG. 26 (See also FIG. 16). The miR-1 target sequence is expected to control viral replication in muscle cells and the miR-122 target sequence is expected to control viral replication in liver cells. miRNA-attenuated SVV and WT (control) SVV viruses were produced by isolation of virus from supernatants of 293T cells transfected with an SVV-encoding plasmid, as described in Example 4. This virus was used to infect permissive H1299 cells expressing miR-1 and miR-122 mimics. After 48 hours, miRNA attenuation of the SVV miR-T construct compared to WT SVV was determined by assessing viral titers in the H446 supernatants with a Cell Titer Glo assay. As shown in Table 9 in the left column below, the negative control mimic, miR-1, and miR-122 TCID₅₀/mL are equivalent, thus the cognate miRNAs had no effect on the viral replication in the case of the WT virus. However, the IC₅₀ of the SVV miR-T (right column) was greatly reduced relative the SVV WT virus (left column) when target cells were transfected with miR-1 or miR-122 mimics, as a multiple log reduction of infectious titers was observed when either miR-1 or miR-122 expressing cells were infected with the SVV miR-T construct. These data demonstrate that virus produced from the self-replicating polynucleotides described herein can be attenuated by insertion of multiple tissue specific miRNAs.

TABLE 9
TCID₅₀/mL values after miRNA mimic pre-treatment

| | SVV WT | SVV miR-T |
|---|---|---|
| Viral input | 7.94e03 | 3.16e03 |
| Negative control mimic | 5.01e07 | 2.00e07 |
| miR-1 mimic | 7.94e07 | 3.16e04 |
| miR-122 mimic | 5.01e07 | 1.26e04 |

### Example 7

### Plasmids Comprising SVV-Encoding Polynucleotides Produce Infectious Virus In Vivo

Experiments were performed to determine the ability of plasmids comprising SVV-encoding polynucleotides to produce infectious virus in vivo using an H1299 xenograft model. Briefly, 5×10⁶ H1299 cells were inoculated subcutaneously in the right flank of 8-week old female athymic nude mice (Charles River Laboratories). When tumor volume reached the volume of approx. 100 mm³, mice were randomly assigned into 2 experimental groups and treated as described hereinafter.

Plasmids comprising an SVV-encoding, ribozyme-enabled expression cassette (SVV w/R) and non-ribozyme enabled (SVV w/o R) cassette exemplified in FIG. 22 were formulated with Lipofectamine 3000. Briefly, 14 μg of each construct were mixed at a 1:1 ratio with Lipofectamine 3000 and vortexed, and then incubated for 10 minutes prior to injection. Two doses of plasmid DNA at 14 μg/dose were administered intratumorally on day 18 and day 20 post-innoculation. Tumor volume was measured 3 times per week using electronic calipers. On days 20, 22, and 23, tumors were harvested for assessment of infectious virus.

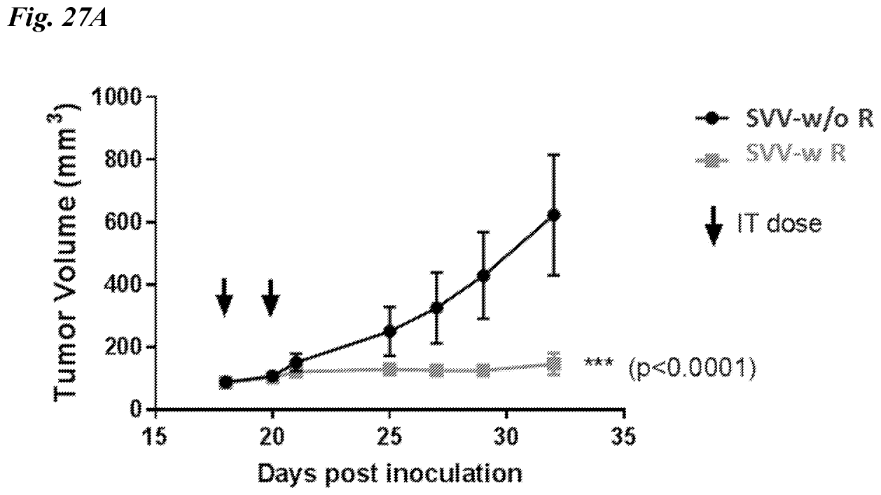

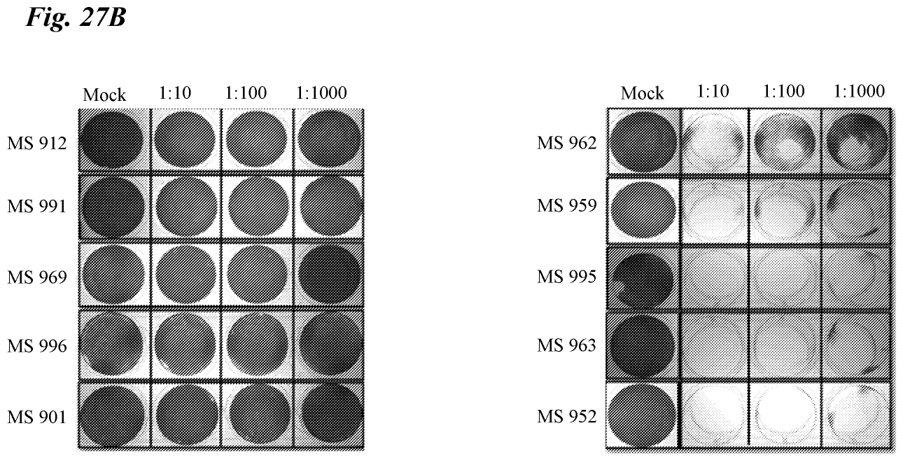

As shown in FIG. 27A, mice treated with ribozyme-enabled SVV-encoding plasmids demonstrated a significant inhibition of tumor growth compared to mice treated with non-ribozyme enabled SVV-encoding plasmids. Virus was isolated from tumors harvested from each group and titrated onto H1299 cells and viral lysis was assessed by crystal violet staining. As shown in FIG. 27B, isolates from the tumors derived from mice treated with the SVV w/ R plasmids contained active, lytic virus, demonstrated by reduced opacity in the crystal violet staining (right panel, FIG. 27B) compared to the virus isolated from the SVV w/o R group (left panel, FIG. 27B). These data demonstrate that plasmids comprising SVV-encoding, ribozyme-enabled polynucleotides produce infectious, lytic virus in vivo and inhibit tumor growth when delivered intratumorally.

### Example 8

### Plasmids Comprising SVV-Encoding Polynucleotides Express Payloads In Vivo

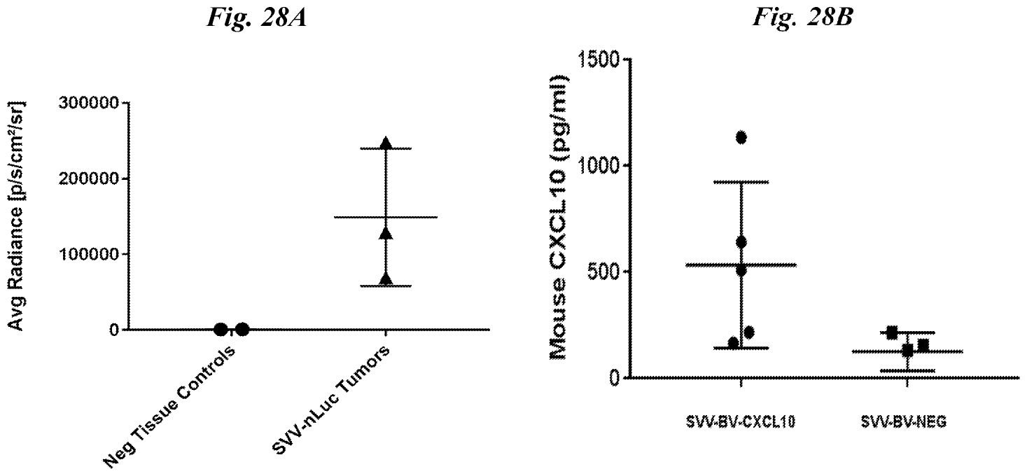

Additional experiments were performed to assess the ability of plasmids comprising SVV-encoding polynucleotides to express various payloads when administered in vivo. Ribozyme-enabled plasmid DNA constructs were formulated and injected intratumorally in an H1299 xenograft model as described in Example 7. In addition to the SVV-encoding polynucleotide sequence, sequences encoding Nanluciferase (FIG. 28A) or CXCL10 (FIG. 28B) were incorporated into the plasmid insert. On day 2 (Nanluciferase) or day 6 (CXCL10), tumors were harvested and assessed for expression of the respective payload proteins. As shown in FIG. 28A-FIG. 28B, intratumoral administration of SVV plasmids with luciferase-encoding polynucleotides, or SVV plasmids with CXCL10-encoding polynucleotides resulted in detection of each payload in isolated tumors (FIG. 28A shows enhanced luminescence and FIG. 28B shows elevated levels of CXCL10). These data demonstrate that, in addition to the production of infectious virus, SVV-encoding plasmids are capable of expression exogenous enzymatic and cytokine payloads in vivo.

### Example 9

### Formulation of Lipid Nanoparticles for Intravenous Delivery of SVV-Encoding Plasmids

SVV-encoding plasmids were formulated in lipid nanoparticles for intravenous delivery of the plasmids.

### Lipid Nanoparticle Production:

Lipids:

The following lipids were used in formulation of lipid nanoparticles:

(a) N-[1-(2,3-dioleoyloxy)propyl]-N,N,N-trimethylammonium (DOTAP);

(b) cholesterol;

(c) 1,2-Dilauroyl-sn-glycero-3-phosphoethanolamine (DLPE);

(d) 1,2-distearoyl-sn-glycero-3-phosphoethanolamine-N-[amino(polyethylene glycol) (PEG-DSPE amine)

(e) 1,2-distearoyl-sn-glycero-3-phosphoethanolamine-(polyethylene glycol) (PEG-DSPE).

Formulation:

Lipids were prepared in ethanol at a ratio of 50:35:15 (DOTAP:Cholesterol:DLPE). In some instances, the lipid nanoparticles were also formulated with 0.2% PEG-DSPE or PEG-DSPE amine. Particles were prepared using microfluidic micro mixture (Precision NanoSystems, Vancouver, BC) at a combined flow rate of 2 mL/min (0.5 mL/min for ethanol, lipid mix and 1.5 mL/min for aqueous buffer, plasmid DNA). The resulting particles were washed by tangential flow filtration (TFF) with PBS containing Ca and Mg.

HA Conjugation Procedure:

High molecular weight hyaluronan (HA) (700 KDa (Lifecore Biomedical)) was dissolved in 0.2 M MES buffer (pH 5.5) to a final concentration of 5 mg/mL. The HA mixture was activated with 1-ethyl-3-(3-dimethylaminopropyl)carbodiimide hydrochloride (EDC) and N-hydroxysulfosuccinimide (sulfo-NHS) at a molar ratio of 1:1:6 (HA:EDC:sulfo-NHS). After 30 min of activation, the lipid particles were added and the pH was adjusted to 7.4. The solution was incubated at room temperature for 2 h.

The resulting parameters for each encapsulation formulation are shown below in Table 10.

TABLE 10
Encapsulation Formulation Parameters

| | | 0.2% PEG- | | HA |
|---|---|---|---|---|
| Formulation | DOTAP:Chol:DLPE | DSPE | Lipid:Plasmid | conjugation |
| 52021-1.D | 50:35:15 | No | 5.33:1 | Yes |
| 52021-2.D | 50:35:15 | Yes | 5.33:1 | Yes |
| 52021-3.C | 50:35:15 | Yes, with NH2 | 5.33:1 | No |
| 52021-4.D | 50:35:15 | | | Yes |

### Analysis of Physical Characteristics of Particle Formulations:

For each of the resulting particle formulations described in Table 10, particle size distribution and zeta potential measurements were determined by light scattering using a Malvern Nano-ZS Zetasizer (Malvern Instruments Ltd, Worcestershire, UK). Size measurements were performed in HBS at pH 7.4 and zeta potential measurements were performed in 0.01 M HBS at pH 7.4. Characteristics of the formulations were evaluated prior to HA conjugation and before and after TFF. The results of these evaluations are shown below in Table 11.

TABLE 11
Zetasizer Data for Encapsulation Formulations

| | Before mixed with | | | | | | | | |
|---|---|---|---|---|---|---|---|---|---|
| | HA | | | Before TFF | | | After TFF | | |
| | Z-Avg | | ZP | Z-Avg | | ZP | Z-Avg | | ZP |
| Formulation | (d · nm) | PdI | (mV) | (d · nm) | PdI | (mV) | (d · nm) | PdI | (mV) |
| 52021-1.D | 184.5 | 0.29 | 43.6 | 395.8 | 0.22 | −37.0 | 498.5 | 0.31 | −36.7 |
| 52021-2.D | 174.5 | 0.36 | 35.4 | 341.2 | 0.26 | −35.3 | 489.1 | 0.34 | −34.1 |
| 52021-3.C | 164.0 | 0.34 | 31.8 | | | | | | |
| 52021-4.D | | | | 337.1 | 0.25 | −31.9 | 437.6 | 0.44 | −32.0 |

### Results:

In order to assess the ability of each of the formulations to successfully deliver the plasmid DNA to cells and to produce infectious virus, H1299 cells were transfected with each of the formulations. Plasmid DNA formulated with Lipofectamine was used as a positive control and Lipofectamine alone was used as a negative control. Three days after transfection, supernatants were harvested and the SVV TCID₅₀/mL was calculated by titration of the supernatants onto H466 cells and a Cell Titer Glo viability assay.

TABLE 12
In vitro Activity of Encapsulation Formulations

| Formulation | TCID₅₀/mL |
|---|---|
| 52021-1.D | 5.01e07 |
| 52021-2.D | 7.94e07 |
| 52021-3.C | 5.01e07 |

As shown in Table 12, lipid particle formulations of plasmid DNA were able to deliver the plasmid DNA to cells and resulted in the production of infectious virus, as the TCID₅₀/mL values for the different formulations demonstrate production of infectious virus.

### Example 10

### Intravenous Injection of Plasmid DNA Results in Delivery to Tumor Sites and Inhibition of Tumor Growth

### Intravenous Delivery in Lung Cancer Xenograft Model:

Experiments were performed to determine whether the lipid particle formulation of SVV-encoding plasmid DNA can deliver pDNA to the tumor when administer systemically. Formulation 52021-4D described in Example 9 and Tables 10 was selected and particles were formulated in PBS with a ˜95% active DNA recovery and lipid encapsidation efficiency.

When tumor volume reached the volume of approx. 150 mm3, 100 μL (approximately 27 μg of DNA) of LNP were administered intravenously. PBS was used as a vehicle control. Two additional doses of LNPs or vehicle controls were intravenously administered every other day for a total of 3 doses. Mice were sacrificed 48 Hrs. post last dosed and tumor tissue was collected.

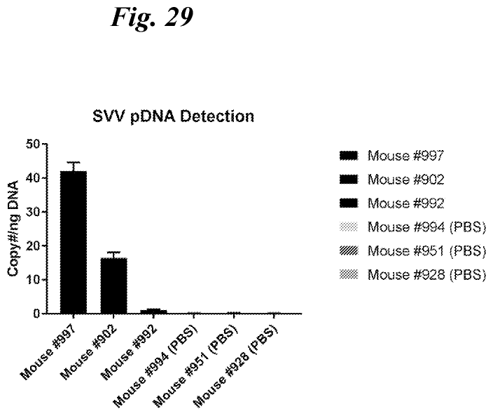

As shown in FIG. 29, SVV plasmid DNA was detected in tumors harvested from mice treated with LNPs. Therefore, the LNPs are able to delivery plasmid DNA to tumor sites.

Experiments were performed to determine whether the lipid particle formulation of SVV-encoding plasmid DNA could affect tumor growth when administered intravenously in the H1299 xenograft model described in Example 7. Due to the presence of the targeting moiety hyaluronic acid and function in vitro, the lipid nanoparticle (LNP) formulation 52021-2D described in Example 9 and Tables 10 was selected for further analysis and particles were formulated in PBS with a ˜95% active DNA recovery and lipid encapsidation efficiency.

When tumor volume reached the volume of approx. 150 mm³, 100 μL (approximately 27 μg of DNA) of LNP were administered intravenously. PBS was used as a vehicle control. Three additional doses of LNPs or vehicle controls were intravenously administered every other day for a total of 4 doses. Tumor volume was measured at least twice a week using electronic calipers.

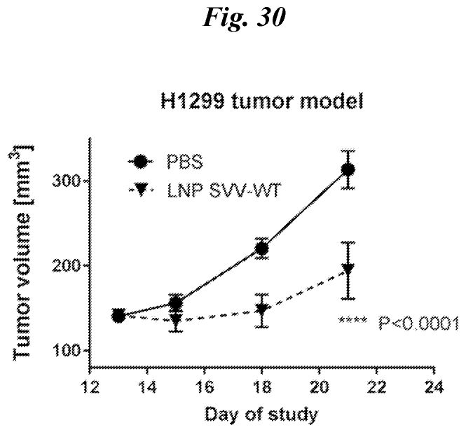

As shown in FIG. 30, intravenous delivery of plasmid DNA formulated in LNPs significantly inhibited tumor growth over time compared to growth observed in PBS controls (FIG. 30, **** p<0.0001, 2-way ANOVA with Bonferroni correction). These results demonstrate that plasmid DNA encoding an infectious virus can be intravenously delivered in a non-viral vehicle, and can significantly inhibit tumor growth in vivo.

### Intravenous Delivery in Hepatocellular Carcinoma Xenograft Model:

Similar experiments will be performed to assess the effect of intravenous LNP delivery in a murine xenograft model of hepatocellular carcinoma. Briefly, mice will be inoculated with a 3×10⁶ HepG2 cells and treated intravenously with LNPs formulated as described above. Tumor growth will be measured over time, and tumors will be harvested at the end of the experiment for further analysis. These experiments are expected to demonstrate the ability of intravenous LNP-encapsulated constructs encoding oncolytic viruses to inhibit tumor growth in a model of hepatocellular carcinoma.

### Example 11

### Treatment of Patients Suffering from Cancer with LNP-Encapsulated Self-Replicating Polynucleotides Encoding Viral Genomes

Experiments can be performed to assess the ability of the self-replicating viral genomes described herein to treat patients suffering from cancer. In such experiments, self-replicating polynucleotides encoding viral genomes are engineered as generally described in Example 1.

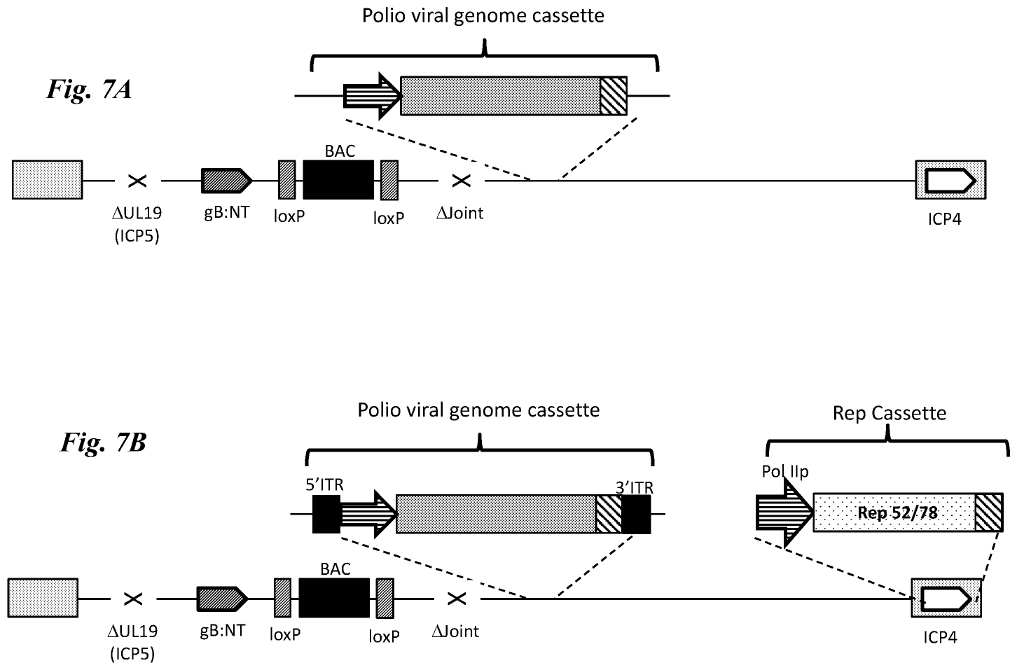

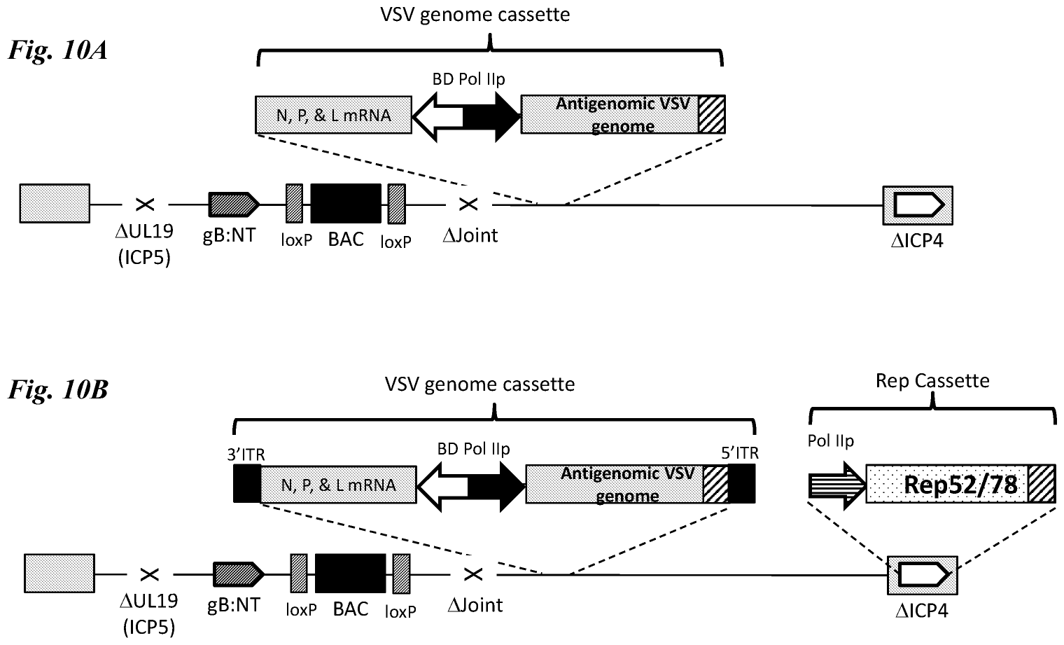

These self-replicating polynucleotides can be further engineered for incorporation into a plasmid backbone. Alternatively, for large scale in vitro propagation of the self-replicating polynucleotides, AAV-ITR sequences can be incorporated to flank the entire viral genome to generate a NanoV construct to aid in polynucleotide replication and nuclear entry. The entire ITR-flanked genome is inserted into an intergenic locus of a recombinant HSV genome backbone (FIG. 4B, FIG. 7B) or alternatively into the ICP4 locus (FIG. 5B, FIG. 10B, ICP4 provided in trans by ICP4 complementing cell line). The AAV rep gene is inserted into ICP0 to enable efficient replication of ITR-flanked viral genome DNA (See Example 3).

Plasmid genomes or NanoV genomes are purified from culture using standard molecular biology techniques (e.g. Maxi-prep) and then encapsulated into lyophilized hyaluronan (HA) surface-modified lipid nanoparticles (LNPs) (See Example 9). Un-encapsulated viral genome DNA is removed by ultracentrifugation and nanoparticle encapsulated viral genomes quantified by qPCR. For in vivo administration to a patient suffering from the cancer, LNPs are prepared in phosphate buffered solution (PBS) along with pharmaceutically acceptable stabilizing agents. The patient is treated on day one with 10¹⁰ vector genomes in a volume of 10 mL pharmaceutically acceptable carrier via intravenous infusion. The patient is monitored using standard of care procedures for presence of cancer. Potential outcomes of these experiments include partial or complete inhibition of tumor growth, inhibition of tumor metastasis, prolonged time in remission, and/or reduced rate of relapse compared to standard of care therapies.

### Example 12

### Treatment of Patients Suffering from Lung Cancer with LNP-Encapsulated Self-Replicating Polynucleotides Encoding Viral Genomes

Experiments can be performed according to Example 11 to assess the ability of the self-replicating viral genomes described herein to treat patients suffering from non-small cell lung cancer (NSCLC) or patients suffering from small cell lung cancer (SCLC).

Exemplary self-replicating polynucleotides that can be encapsulated in LNPs and used in the treatment of NSCLC and SCLC are outlined below in Table 13.

TABLE 13
Summary of self-replicating vectors for treatment of NSCLC and SCLC

| | | miR-T insert | | Pay load insert | |
|---|---|---|---|---|---|
| Virus | miR-T | location | Payload | location | Vector |
| Polio virus | miR-124 | 3′ UTR of | +/− | | ITR-flanked NanoV |
| | miR-145 | genome | | | construct |
| | miR-34a | | | | |
| | let7 | | | | |
| VSV | miR-124 | N, P M, | +/− | | ITR-flanked NanoV |
| | miR-143 | and/or L | | | construct |
| | miR-145 | | | | |
| | let7 | | | | |
| Adenovirus | miR-124 | E1, E2, E3, | +/− | | ITR-flanked NanoV |
| | miR-143 | and/or E4 | | | construct |
| | miR-145 | | | | |
| | let7 | | | | |
| Coxsackievirus | miR-124 | 3′ UTR of | +/− | In frame linker | Ribozyme-flanked |
| (CVB3) | miR-1 | genome | | between 2A | genome plasmid |
| | | | | and 2B | |
| SVV | | | +/− | In frame linker | Ribozyme-flanked |
| | | | | between 2A | genome plasmid |
| | | | | and 2B | |
| Polio virus | miR-124 | 3′ UTR of | +/− | | Genome plasmid |
| | miR-145 | genome | | | |
| | miR-34a | | | | |
| | let7 | | | | |
| VSV | miR-124 | N, P M, | +/− | | Genome plasmid |
| | miR-143 | and/or L | | | |
| | miR-145 | | | | |
| | let7 | | | | |
| Adenovirus | miR-124 | E1, E2, E3, | +/− | | Genome plasmid |
| | miR-143 | and/or E4 | | | |
| | miR-145 | | | | |
| | let7 | | | | |

### Example 13

### Treatment of a Patient Suffering from Hepatocellular Carcinoma

Experiments can be performed according to Example 11 to assess the ability of the self-replicating viral genomes described herein to treat patients suffering from hepatocellular carcinoma.

Exemplary self-replicating polynucleotides that can be encapsulated in LNPs and used in the treatment of hepatocellular carcinoma are outlined below in Table 14.

TABLE 14
Summary of self-replicating vectors for treatment of Hepatocellular Carcinoma

| | | miR-T insert | | Payload insert | |
|---|---|---|---|---|---|
| Virus | miR-T | location | Payload | location | Vector |
| Polio virus | miR-124 | 3′ UTR of | +/− | | ITR-flanked NanoV |
| | miR-145 | genome | | | construct |
| | miR-34a | | | | |
| | let7 | | | | |
| VSV | miR-122 | N, P M, | +/− | | ITR-flanked NanoV |
| | miR-124 | and/or L | | | construct |
| | miR-34a | | | | |
| | let7 | | | | |
| Adenovirus | miR-122 | E1, E2, E3, | +/− | | ITR-flanked NanoV |
| | miR-124 | and/or E4 | | | construct |
| | miR-34a | | | | |
| | let7 | | | | |
| Coxsackievirus | | 3′ UTR of | +/− | In frame linker | Ribozyme-flanked |
| (CVB3) | | genome | | between 2A | genome plasmid |
| | | | | and 2B | |
| SVV | | | +/− | In frame linker | Ribozyme-flanked |
| | | | | between 2A | genome plasmid |
| | | | | and 2B | |
| Polio virus | miR-124 | 3′ UTR of | +/− | | Genome plasmid |
| | miR-145 | genome | | | |
| | miR-34a | | | | |
| | let7 | | | | |
| VSV | miR-122 | N, P M, | +/− | | Genome plasmid |
| | miR-124 | and/or L | | | |
| | miR-34a | | | | |
| | let7 | | | | |
| Adenovirus | miR-122 | E1, E2, E3, | +/− | | Genome plasmid |
| | miR-124 | and/or E4 | | | |
| | miR-34a | | | | |
| | let7 | | | | |

### Example 14

### Treatment of a Patient Suffering from Prostate Cancer

Experiments can be performed according to Example 11 to assess the ability of the self-replicating viral genomes described herein to treat patients suffering from prostate cancer.

Exemplary self-replicating polynucleotides that can be encapsulated in LNPs and used in the treatment of prostate cancer are outlined below in Table 15.

TABLE 15
Summary of self-replicating vectors for treatment of Prostate Cancer

| | | miR-T insert | | Payload insert | |
|---|---|---|---|---|---|
| Virus | miR-T | location | Payload | location | Vector |
| Polio virus | miR-124 | 3′ UTR of | +/− | | ITR-flanked NanoV |
| | miR-143 | genome | | | construct |
| | miR-145 | | | | |
| | let7 | | | | |
| VSV | miR-124 | N, P M, | +/− | | ITR-flanked NanoV |
| | miR-143 | and/or L | | | construct |
| | miR-145 | | | | |
| | let7 | | | | |
| Adenovirus | miR-124 | E1, E2, E3, | +/− | | ITR-flanked NanoV |
| | miR-145 | and/or E4 | | | construct |
| | miR-34a | | | | |
| | let7 | | | | |
| Coxsackievirus | | 3′ UTR of | +/− | In frame linker | Ribozyme-flanked |
| (CVB3) | | genome | | between 2A | genome plasmid |
| | | | | and 2B | |
| SVV | | | +/− | In frame linker | Ribozyme-flanked |
| | | | | between 2A | genome plasmid |
| | | | | and 2B | |
| Polio virus | miR-124 | 3′ UTR of | +/− | | Genome plasmid |
| | miR-143 | genome | | | |
| | miR-145 | | | | |
| | let7 | | | | |
| VSV | miR-124 | N, P M, | +/− | | Genome plasmid |
| | miR-143 | and/or L | | | |
| | miR-145 | | | | |
| | let7 | | | | |
| Adenovirus | miR-124 | E1, E2, E3, | +/− | | Genome plasmid |
| | miR-145 | and/or E4 | | | |
| | miR-34a | | | | |
| | let7 | | | | |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

TABLE 1
Summary of relationships between 12 select oncomiRs (9 tumor
suppressors and 3 oncogenic miRNAs) and various cancers

| | Down-regulated | | | | | | | | | Up-regulated | | |
|---|---|---|---|---|---|---|---|---|---|---|---|---|
| | | miR- | miR- | miR- | miR- | miR- | miR- | miR- | miR- | miR- | miR- | miR- |
| Malignancy | let-7 | 15a | 16 | 29a | 34a | 98 | 101 | 124 | 202 | 17 | 21 | 155 |
| acute lymphoblastic leukemia | X | | | | | | | X | | | | |
| acute myeloid leukemia | X | | | X | | | | | | X | | X |
| acute promyelocytic leukemia | X | | | | | | | | | | | |
| adrenal cortical carcinoma | | | | | | | | | | | X | |
| anaplastic astrocytoma | | | | | | | | X | | | | |
| anaplastic large-cell lymphoma | | | | | | | | | | | | X |
| astrocytoma | | | | | | | | X | | | | |
| B cell lymphoma | | | | | X | | | | | X | | |
| bladder cancer | | | X | | X | | X | X | | X | | X |
| breast cancer | X | X | X | X | X | X | | X | | X | | X |
| breast carcinoma | | | | | | | | | | X | | |
| bronchioloalveolar carcinoma | X | | | | | X | | | | | | |
| cervical cancer | | | | | | | | X | | X | | X |
| cervical carcinoma | | X | X | | X | | X | | | | | |
| cervical squamous cell | | | | X | | | | X | | | | |
| carcinoma | | | | | | | | | | | | |
| cholangiocarcinoma | | | | | X | | X | | | X | | |
| chondrosarcoma | X | | | | | | | | | | | |
| chordoma | | | | | X | | | | | | | |
| choriocarcinoma | | | | | X | | | | | | | |
| chronic lymphocytic leukemia | | X | X | | | | | | | | | X |
| chronic myelogenous leukemia | | | X | | | | | | | | | X |
| clear cell renal cell cancer | | | | | X | | | | | | | X |
| colon cancer | X | | | | X | X | X | | | | | X |
| colorectal cancer | X | X | X | X | X | | X | X | X | X | | X |
| colorectal carcinoma | | | | | | | | | | X | | X |
| cutaneous T cell lymphoma | | | | | | | | | | | | X |
| diffuse large B cell lymphoma | | | | | | | | | | | | X |
| endometrial cancer | | | | | X | | X | | | | | X |
| epithelial ovarian cancer | | | | | | | | X | | | | |
| esophageal cancer | | X | | | | | X | X | | | | |
| esophageal squamous cell | X | | X | | X | X | X | | | X | | |
| carcinoma | | | | | | | | | | | | |
| extrahepatic | | | | | X | | | | | | | |
| cholangiocarcinoma | | | | | | | | | | | | |
| follicular lymphoma | | | | | | | | | X | | | |
| gallbladder carcinoma | | | | | | | | | | | | X |
| gastric cancer | X | | | X | X | X | X | X | X | X | | X |
| glioblastoma | X | | | | X | | X | X | | | | |
| glioma | X | | X | | X | X | | X | | X | | X |
| head and neck cancer | | | | | | | | | | | | |
| head and neck squamous cell | X | | X | X | X | | | | | | X | |
| carcinoma | | | | | | | | | | | | |
| hepatocellular carcinoma | X | | X | X | X | X | X | X | X | X | X | X |
| hypopharyngeal squamous | | | | | | | | | | | X | |
| cell carcinoma | | | | | | | | | | | | |
| kidney cancer | | | | | | | | | | | X | |
| laryngeal carcinoma | | | X | | | | | | | | X | |
| laryngeal squamous cell | | | | | | | X | | | | X | |
| carcinoma | | | | | | | | | | | | |
| liver cancer | | | | | | | X | | | | X | X |
| lung adenocarcinoma | | | X | | | | | | | | | X |
| lung cancer | X | X | X | | X | X | X | | | X | X | X |
| malignant melanoma | X | | | | X | X | X | | | X | X | X |
| malt lymphoma | | | | | | | | | | | | X |
| mantle cell lymphoma | | | | X | | | | X | | X | | X |
| medulloblastoma | | | | | | | | X | | X | | |
| mesenchymal cancer | | | | X | | | | | | | | |
| monocytic leukemia | | | | X | | | | | | | | |
| multiple myeloma | | | | | | | | | | | X | |
| nasopharyngeal cancer | | | | | | | | | | X | | |
| nasopharyngeal carcinoma | X | | | | | X | X | X | | | X | X |
| neuroblastoma | X | X | X | X | X | X | | X | | | | |
| non-small cell lung cancer | X | X | X | X | X | | X | X | | X | X | X |
| oral cancer | X | | | | X | | | | | | X | |
| oral squamous cell carcinoma | | | | X | | | | X | | | X | X |
| osteosarcoma | X | X | X | | X | | X | X | X | X | X | |
| ovarian cancer | X | | | | X | X | | X | | | X | X |
| ovarian carcinoma | | | | | | | X | | | | | |
| pancreatic adenocarcinoma | | | | | X | | | | | | X | |
| pancreatic cancer | | X | | | | | X | X | | X | X | |
| pancreatic ductal | X | X | X | | X | X | | | | | X | |
| adenocarcinoma | | | | | | | | | | | | |
| papillary thyroid carcinoma | X | | X | | X | | X | | | | X | X |
| pituitary carcinoma | | | | | | | | | | X | | |
| prostate cancer | X | X | X | | X | | X | X | | | X | |
| rectal cancer | | | | | X | | | | | | X | X |
| renal cell carcinoma | X | | X | | X | | | | | | X | |
| renal clear cell carcinoma | X | | | | | | | | | | | X |
| retinoblastoma | | | | | X | | X | | | | X | |
| squamous carcinoma | | X | X | | X | | | | | | X | X |
| T cell lymphoblastic lymphoma | | | | | | | | | | X | | |
| uveal melanoma | | | | | X | | | | | | | |

TABLE 2
Summary of oncomiRs and cancers

| Malignancy | Down-regulated miRs | Up-regulated miRs |
|---|---|---|
| breast cancer | let-7a, let-7a-1, let-7a-2, let-7a-3, let- | mir-10b, mir-125a, mir-135a, |
| | 7b, let-7c, let-7d, let-7e, let-7f-1, let- | mir-140, mir-141, mir-142, |
| | 7f-2, let-7g, let-7i, mir-100, mir-107, | mir-150, mir-155, mir-181a, |
| | mir-10a, mir-10b, mir-122, mir-124, | mir-181b, mir-182, mir-18a, |
| | mir-1258, mir-125a-5p, mir-125b. | mir-18b, mir-191, mir-196a, |
| | mir-126, mir-127, mir-129, mir-130a, | mir-197, mir-19a, mir-19b, |
| | mir-132, mir-133a, mir-143, mir-145, | mir-200a, mir-200b, mir-200c, |
| | mir-146a, mir-146b, mir-147, mir- | mir-203, mir-205, mir-20a, |
| | 148a, mir-149, mir-152, mir-153, | mir-20b, mir-21, mir-217, mir- |
| | mir-15a, mir-16, mir-17-5p, mir- | 221, mir-224, mir-23a, mir-24, |
| | 181a, mir-1826, mir-183, mir-185, | mir-24-2-5p, mir-24-3p, mir- |
| | mir-191, mir-193a-3p, mir-193b, mir- | 27a, mir-29a, mir-29b-1, mir- |
| | 195, mir-199b-5p, mir-19a-3p, mir- | 29b-2, mir-29c, mir-373, mir- |
| | 200a, mir-200b, mir-200c, mir-205, | 378, mir-423, mir-429, mir- |
| | mir-206, mir-211, mir-216b, mir-218, | 495, mir-503, mir-510, mir- |
| | mir-22, mir-26a, mir-26b, mir-300, | 520c, mir-526b, mir-96 |
| | mir-30a, mir-31, mir-335, mir-339- | |
| | 5p, mir-33b, mir-34a, mir-34b, mir- | |
| | 34c, mir-374a, mir-379, mir-381, mir- | |
| | 383, mir-425, mir-429, mir-450b-3p, | |
| | mir-494, mir-495, mir-497, mir-502- | |
| | 5p, mir-517a, mir-574-3p, mir-638, | |
| | mir-7, mir-720, mir-7515, mir-92a, | |
| | mir-98, mir-99a, mmu-mir-290-3p, | |
| | mmu-mir-290-5p | |
| chondrosarcoma | let-7a, mir-100, mir-136, mir-145, | |
| | mir-199a, mir-222, mir-30a, mir-335, | |
| | mir-376a | |
| colorectal cancer | let-7a, mir-1, mir-100, mir-101, mir- | let-7a, mir-103, mir-106a, mir- |
| | 124, mir-125a, mir-126, mir-129, | 10b, mir-1179, mir-1229, mir- |
| | mir-1295b-3p, mir-1307, mir-130b, | 1246, mir-125b-2*, mir-1269a, |
| | mir-132, mir-133a, mir-133b, mir- | mir-130b, mir-133b, mir-135a, |
| | 137, mir-138, mir-139, mir-139-5p, | mir-135a-1, mir-135a-2, mir- |
| | mir-140-5p, mir-143, mir-145, mir- | 135b, mir-139-3p, mir-145, |
| | 148a, mir-148b, mir-149, mir-150-5p, | mir-150, mir-150*, mir-155, |
| | mir-154, mir-15a, mir-15b, mir-16, | mir-17, mir-181a, mir-182, |
| | mir-18a, mir-191, mir-192, mir-193a- | mir-183, mir-18a, mir-191, |
| | 5p, mir-194, mir-195, mir-196a, mir- | mir-196a, mir-196b, mir-19a, |
| | 198, mir-199a-5p, mir-200c, mir-203, | mir-19b, mir-200b, mir-200c, |
| | mir-204-5p, mir-206, mir-212, mir- | mir-203, mir-204-5p, mir-20a, |
| | 215, mir-218, mir-22, mir-224, mir- | mir-20a-5p, mir-21, mir-210, |
| | 24-3p, mir-26b, mir-27a, mir-28-3p, | mir-211, mir-221, mir-223, |
| | mir-28-5p, mir-29b, mir-30a-3p, mir- | mir-224, mir-23a, mir-25, mir- |
| | 30b, mir-320a, mir-328, mir-338-3p, | 27a, mir-29a, mir-301a, mir- |
| | mir-342, mir-345, mir-34a, mir-34a- | 31, mir-32, mir-320b, mir-326, |
| | 5p, mir-361-5p, mir-375, mir-378, | mir-424, mir-429, mir-494, |
| | mir-378a-3p, mir-378a-5p, mir-409- | mir-497, mir-499-5p, mir-592, |
| | 3p, mir-422a, mir-4487, mir-483, | mir-630, mir-7-5p, mir-892a, |
| | mir-497, mir-498, mir-518a-3p, mir- | mir-92, mir-92a, mir-93, mir- |
| | 551a, mir-574-5p, mir-625, mir-638, | 95, mir-96 |
| | mir-7, mir-96-5p | |
| esophageal | let-7a, let-7a-1, let-7a-2, let-7a-3, let- | mir-100, mir-1179, mir-1290, |
| squamous cell | 7b, let-7c, let-7d, let-7e, let-7f-1, let- | mir-130b, mir-145, mir-16, |
| carcinoma | 7f-2, let-7g, let-7i, mir-1, mir-100, | mir-17, mir-183, mir-18a, mir- |
| | mir-101, mir-126, mir-1294, mir- | 19a, mir-19b, mir-208, mir- |
| | 133a, mir-133b, mir-138, mir-143, | 20a, mir-21, mir-218, mir-223, |
| | mir-145, mir-150, mir-185, mir-195, | mir-25, mir-30a-5p, mir-31, |
| | mir-200b, mir-203, mir-21, mir-210, | mir-330-3p, mir-373, mir-9, |
| | mir-214, mir-218, mir-22, mir-27a, | mir-92a, mir-942 |
| | mir-29b, mir-29c, mir-302b, mir-34a, | |
| | mir-375, mir-494, mir-518b, mir-655, | |
| | mir-98, mir-99a | |
| gastric cancer | let-7a, let-7b, let-7g, mir-1, mir-101, | mir-100, mir-103, mir-106a, |
| | mir-103a, mir-10a, mir-10b, mir- | mir-106b, mir-107, mir-10a, |
| | 1207-5p, mir-122, mir-1228*, mir- | mir-10b, mir-1259, mir-125b, |
| | 124, mir-124-3p, mir-125a-3p, mir- | mir-126, mir-1274a, mir-1303, |
| | 126, mir-1266, mir-1271, mir-129-1- | mir-130b*, mir-135a-5p, mir- |
| | 3p, mir-129-2-3p, mir-129-3p, mir- | 135b, mir-138, mir-143, mir- |
| | 129-5p, mir-133a, mir-133b, mir-137, | 146a, mir-147, mir-148a, mir- |
| | mir-141, mir-143, mir-144, mir-145, | 150, mir-17, mir-17-5p, mir- |
| | mir-146a, mir-146a-5p, mir-148a, | 181a, mir-181a-2*, mir-181a- |
| | mir-148b, mir-149, mir-152, mir-155, | 5p, mir-181c, mir-183, mir- |
| | mir-155-5p, mir-181a, mir-181b, mir- | 185, mir-18a, mir-191, mir- |
| | 182, mir-183, mir-185, mir-194, mir- | 192, mir-196a, mir-196a*, |
| | 195, mir-197, mir-199a-3p, mir-200b, | mir-196a-5p, mir-196b, mir- |
| | mir-200c, mir-202-3p, mir-204, mir- | 199a, mir-199a-3p, mir-199a- |
| | 204-5p, mir-205, mir-206, mir-210, | 5p, mir-19a, mir-19b, mir- |
| | mir-212, mir-217, mir-218, mir-22, | 200b, mir-20a, mir-21, mir- |
| | mir-23b, mir-24, mir-26a, mir-29a, | 214, mir-215, mir-221, mir- |
| | mir-29a-3p, mir-29b, mir-29b-1, mir- | 221*, mir-222, mir-223, mir- |
| | 29b-2, mir-29c, mir-30a-5p, mir-30b, | 224, mir-23a, mir-23b, mir- |
| | mir-31, mir-328, mir-329, mir-331- | 27a, mir-27b, mir-296-5p, mir- |
| | 3p, mir-335-5p, mir-338, mir-338-3p, | 301a, mir-302f, mir-337-3p, |
| | mir-34a, mir-34b, mir-34c, mir-361- | mir-340*, mir-34a, mir-362- |
| | 5p, mir-367, mir-375, mir-378, mir- | 3p, mir-370, mir-374a, mir- |
| | 409-3p, mir-410, mir-429, mir-433, | 377, mir-421, mir-425, mir- |
| | mir-449, mir-449a, mir-490-3p, mir- | 500, mir-520c-3p, mir-544, |
| | 494, mir-497, mir-503, mir-506, mir- | mir-575, mir-601, mir-616*, |
| | 513b, mir-520d-3p, mir-542-3p, mir- | mir-650, mir-92, mir-98, mir- |
| | 622, mir-625, mir-638, mir-663, mir- | 99a |
| | 7, mir-765, mir-9 | |
| glioma | let-7a, let-7f, mir-106a, mir-107, mir- | mir-106b, mir-106b-5p, mir- |
| | 122, mir-124, mir-124-5p, mir-124a, | 10b, mir-125b, mir-132, mir- |
| | mir-125b, mir-128, mir-136, mir-137, | 155, mir-17, mir-181a, mir- |
| | mir-139, mir-143, mir-145, mir-146a, | 182, mir-183, mir-193b, mir- |
| | mir-146b, mir-146b-5p, mir-152, mir- | 19a, mir-19b, mir-20a, mir- |
| | 15b, mir-16, mir-181a, mir-181a-1, | 210, mir-214, mir-221, mir- |
| | mir-181a-2, mir-181b, mir-181b-1, | 222, mir-224, mir-23a, mir-24, |
| | mir-181b-2, mir-181c, mir-181d, mir- | mir-24-3p, mir-25, mir-26a, |
| | 184, mir-185, mir-195, mir-199a-3p, | mir-27a-3p, mir-27b, mir-30a- |
| | mir-200a, mir-200b, mir-203, mir- | 5p, mir-30e, mir-30e*, mir- |
| | 204, mir-205, mir-218, mir-219-5p, | 328, mir-335, mir-33a, mir- |
| | mir-23b, mir-26b, mir-27a, mir-29c, | 372, mir-486, mir-494, mir- |
| | mir-320, mir-326, mir-328, mir-34a, | 497, mir-566, mir-603, mir- |
| | mir-34c-3p, mir-34c-5p, mir-375, | 650, mir-675, mir-9, mir-92b, |
| | mir-383, mir-451, mir-452, mir-483- | mir-93, mir-96 |
| | 5p, mir-495, mir-584, mir-622, mir- | |
| | 656, mir-7, mir-98 | |
| nasopharyngeal | let-7a, let-7a-1, let-7a-2, let-7a-3, let- | mir-10b, mir-144, mir-149, |
| carcinoma | 7b, let-7c, let-7d, let-7e, let-7f-1, let- | mir-155, mir-18a, mir-21, mir- |
| | 7f-2, let-7g, let-7i, mir-1, mir-101, | 214, mir-24, mir-421, mir-663, |
| | mir-124, mir-138, mir-143, mir-145, | mir-7-5p, mir-93 |
| | mir-148a, mir-200b, mir-204, mir- | |
| | 216b, mir-29c, mir-320a, mir-324-3p, | |
| | mir-34c, mir-375, mir-378, mir-451, | |
| | mir-506, mir-9, mir-98 | |
| non-small cell lung | let-7a, let-7c, mir-1, mir-100, mir- | mir-10b, mir-125a-5p, mir- |
| cancer | 101, mir-106a, mir-107, mir-124, | 1280, mir-136, mir-140, mir- |
| | mir-125a-3p, mir-125a-5p, mir-126*, | 141, mir-142-3p, mir-145, |
| | mir-129, mir-133a, mir-137, mir-138, | mir-146a, mir-150, mir-18a, |
| | mir-140, mir-143, mir-145, mir-146a, | mir-196a, mir-19a, mir-200a, |
| | mir-146b, mir-148a, mir-148b, mir- | mir-200c, mir-205, mir-205- |
| | 149, mir-152, mir-153, mir-154, mir- | 5p, mir-21, mir-212, mir-22, |
| | 155, mir-15a, mir-16, mir-17-5p, mir- | mir-221, mir-222, mir-24, mir- |
| | 181a-1, mir-181a-2, mir-181b, mir- | 25, mir-29c, mir-31, mir-328, |
| | 181b-1, mir-181b-2, mir-181c, mir- | mir-330-3p, mir-339, mir-34a, |
| | 181d, mir-184, mir-186, mir-193b, | mir-375, mir-494, mir-675-5p, |
| | mir-195, mir-199a, mir-204, mir-212, | mir-9, mir-92b, mir-93, mir-95 |
| | mir-221, mir-224, mir-26b, mir-27a, | |
| | mir-27b, mir-29a, mir-29b, mir-29c, | |
| | mir-30a, mir-30b, mir-30c, mir-30d, | |
| | mir-30d-5p, mir-30e-5p, mir-32, mir- | |
| | 335, mir-338-3p, mir-340, mir-342- | |
| | 3p, mir-34a, mir-34b, mir-361-3p, | |
| | mir-365, mir-373, mir-375, mir-429, | |
| | mir-449a, mir-4500, mir-451, mir- | |
| | 4782-3p, mir-497, mir-503, mir-512- | |
| | 3p, mir-520a-3p, mir-526b, mir-625*, | |
| | mir-96, mir-99a | |
| osteosarcoma | let-7a, mir-1, mir-100, mir-101, mir- | mir-128, mir-151-3p, mir-17, |
| | 122, mir-124, mir-125b, mir-126, | mir-181a, mir-181b, mir-181c, |
| | mir-127-3p, mir-132, mir-133a, mir- | mir-18a, mir-191, mir-195-5p, |
| | 141, mir-142-3p, mir-142-5p, mir- | mir-199a-3p, mir-19a, mir- |
| | 143, mir-144, mir-145, mir-153, mir- | 19b, mir-20a, mir-21, mir-210, |
| | 16, mir-183, mir-194, mir-195, mir- | mir-214, mir-221, mir-27a, |
| | 199a-3p, mir-204, mir-212, mir-217, | mir-300, mir-320a, mir-374a- |
| | mir-218, mir-22, mir-23a, mir-24, | 5p, mir-720, mir-9, mir-92a |
| | mir-26a, mir-26b, mir-29b, mir-32, | |
| | mir-320, mir-335, mir-33b, mir-340, | |
| | mir-34a, mir-34b, mir-34c, mir-375, | |
| | mir-376c, mir-382, mir-3928, mir- | |
| | 424, mir-429, mir-449a, mir-451, | |
| | mir-454, mir-503, mir-519d, mir-646 | |
| pancreatic ductal | let-7a, let-7a-1, let-7a-2, let-7a-3, let- | mir-10b, mir-186, mir-18a, |
| adenocarcinoma | 7b, let-7c, let-7d, let-7e, let-7f-1, let- | mir-192, mir-194, mir-196a, |
| | 7f-2, let-7g, let-7i, mir-126, mir-135a, | mir-198, mir-203, mir-21, mir- |
| | mir-143, mir-144, mir-145, mir-148a, | 212, mir-30b-5p, mir-31, mir- |
| | mir-150, mir-15a, mir-16, mir-200a, | 34a, mir-369-5p, mir-376a, |
| | mir-200b, mir-200c, mir-217, mir- | mir-541 |
| | 218, mir-337, mir-375, mir-494, mir- | |
| | 615-5p, mir-98 | |
| renal cell carcinoma | let-7a, let-7d, mir-1, mir-106a*, mir- | mir-100, mir-1233, mir-1260b, |
| | 126, mir-1285, mir-129-3p, mir-1291, | mir-146a, mir-146b, mir-16, |
| | mir-133a, mir-133b, mir-135a, mir- | mir-193a-3p, mir-203a, mir- |
| | 138, mir-141, mir-143, mir-145, mir- | 21, mir-210, mir-27a, mir-362, |
| | 182-5p, mir-199a-3p, mir-200a, mir- | mir-572, mir-7 |
| | 205, mir-218, mir-28-5p, mir-30a, | |
| | mir-30c, mir-30d, mir-34a, mir-378, | |
| | mir-429, mir-509-3p, mir-509-5p, | |
| | mir-646 | |
| bronchioloalveolar | let-7a-1, let-7a-2, let-7a-3, let-7b, let- | |
| carcinoma | 7c, let-7d, let-7e, let-7f-1, let-7f-2, | |
| | let-7g, let-7i, mir-98 | |
| colon cancer | let-7a-1, let-7a-2, let-7a-3, let-7b, let- | mir-1290, mir-145, mir-155, |
| | 7c, let-7d, let-7e, let-7f-1, let-7f-2, | mir-181a, mir-18a, mir-200c, |
| | let-7g, let-7i, mir-100, mir-101, mir- | mir-31, mir-675 |
| | 126, mir-142-3p, mir-143, mir-145, | |
| | mir-192, mir-200c, mir-21, mir-214, | |
| | mir-215, mir-25, mir-302a, mir-320, | |
| | mir-320a, mir-34a, mir-34c, mir-365, | |
| | mir-373, mir-424, mir-429, mir-455, | |
| | mir-484, mir-502, mir-503, mir-93, | |
| | mir-98 | |
| hepatocellular | let-7a-1, let-7a-2, let-7a-3, let-7b, let- | mir-106b, mir-10b, mir-122, |
| carcinoma | 7c, let-7d, let-7e, let-7f, let-7f-1, let- | mir-1228, mir-1269, mir-128a, |
| | 7f-2, let-7g, let-7i, mir-1, mir-100, | mir-130a, mir-130b, mir-146a, |
| | mir-101, mir-105, mir-122, mir-122a, | mir-153, mir-155, mir-17-5p, |
| | mir-1236, mir-124, mir-125b, mir- | mir-181a, mir-181a-1, mir- |
| | 126, mir-127, mir-1271, mir-128-3p, | 181a-2, mir-181b, mir-181b-1, |
| | mir-129-5p, mir-130a, mir-130b, mir- | mir-181b-2, mir-181c, mir- |
| | 133a, mir-134, mir-137, mir-138, | 181d, mir-182, mir-183, mir- |
| | mir-139, mir-139-5p, mir-140-5p, | 184, mir-190b, mir-191, mir- |
| | mir-141, mir-142-3p, mir-143, mir- | 20a, mir-20b, mir-21, mir-210, |
| | 144, mir-145, mir-146a, mir-148a, | mir-214, mir-215, mir-216a, |
| | mir-148b, mir-150-5p, mir-15b, mir- | mir-217, mir-221, mir-222, |
| | 16, mir-181a-5p, mir-185, mir-188- | mir-223, mir-224, mir-23a, |
| | 5p, mir-193b, mir-195, mir-195-5p, | mir-24, mir-25, mir-27a, mir- |
| | mir-197, mir-198, mir-199a, mir- | 301a, mir-30d, mir-31, mir- |
| | 199a-5p, mir-199b, mir-199b-5p, mir- | 3127, mir-32, mir-331-3p, |
| | 200a, mir-200b, mir-200c, mir-202, | mir-362-3p, mir-371-5p, mir- |
| | mir-203, mir-204-3p, mir-205, mir- | 372, mir-373, mir-423, mir- |
| | 206, mir-20a, mir-21, mir-21-3p, mir- | 429, mir-452, mir-483-3p, |
| | 211, mir-212, mir-214, mir-217, mir- | mir-483-5p, mir-485-3p, mir- |
| | 218, mir-219-5p, mir-22, mir-26a, | 490-3p, mir-494, mir-495, |
| | mir-26b, mir-29a, mir-29b-1, mir- | mir-500, mir-501-5p, mir- |
| | 29b-2, mir-29c, mir-302b, mir-302c, | 519d, mir-520g, mir-574-3p, |
| | mir-30a, mir-30a-3p, mir-335, mir- | mir-590-5p, mir-630, mir-650, |
| | 338-3p, mir-33a, mir-34a, mir-34b, | mir-657, mir-664, mir-885-5p, |
| | mir-365, mir-370, mir-372, mir-375, | mir-9, mir-92a, mir-96 |
| | mir-376a, mir-377, mir-422a, mir- | |
| | 424, mir-424-5p, mir-433, mir-4458, | |
| | mir-448, mir-450a, mir-451, mir-485- | |
| | 5p, mir-486-5p, mir-497, mir-503, | |
| | mir-506, mir-519d, mir-520a, mir- | |
| | 520b, mir-520c-3p, mir-582-5p, mir- | |
| | 590-5p, mir-610, mir-612, mir-625, | |
| | mir-637, mir-675, mir-7, mir-877, | |
| | mir-940, mir-941, mir-98, mir-99a | |
| lung cancer | let-7a-1, let-7a-2, let-7a-3, let-7b, let- | mir-10b, mir-135b, mir-150, |
| | 7c, let-7d, let-7e, let-7f-1, let-7f-2, | mir-155, mir-17, mir-182, mir- |
| | let-7g, let-7i, mir-1, mir-101, mir- | 183-3p, mir-18a, mir-197, mir- |
| | 133b, mir-138, mir-142-5p, mir-144, | 19a, mir-19b, mir-205, mir- |
| | mir-145, mir-1469, mir-146a, mir- | 20a, mir-21, mir-210, mir-24, |
| | 153, mir-15a, mir-15b, mir-16-1, mir- | mir-30d, mir-4423, mir-5100, |
| | 16-2, mir-182, mir-192, mir-193a-3p, | mir-570, mir-663, mir-7, mir- |
| | mir-194, mir-195, mir-198, mir-203, | 92a |
| | mir-217, mir-218, mir-22, mir-223, | |
| | mir-26a, mir-26b, mir-29c, mir-33a, | |
| | mir-34a, mir-34b, mir-34c, mir-365, | |
| | mir-449a, mir-449b, mir-486-5p, mir- | |
| | 545, mir-610, mir-614, mir-630, mir- | |
| | 660, mir-7-5p, mir-9500, mir-98, mir- | |
| | 99b | |
| neuroblastoma | let-7a-1, let-7a-2, let-7a-3, let-7b, let- | mir-125b, mir-15a, mir-15b, |
| | 7c, let-7d, let-7e, let-7f-1, let-7f-2, | mir-16-1, mir-16-2, mir-18a, |
| | let-7g, let-7i, mir-124, mir-137, mir- | mir-195, mir-19a, mir-23a, |
| | 145, mir-181c, mir-184, mir-200a, | mir-421, mir-92 |
| | mir-29a, mir-335, mir-338-3p, mir- | |
| | 34a, mir-449a, mir-885-5p, mir-98 | |
| prostate cancer | let-7a-3p, let-7c, mir-100, mir-101, | mir-125b, mir-141, mir-153, |
| | mir-105, mir-124, mir-128, mir-1296, | mir-155, mir-181a-1, mir- |
| | mir-130b, mir-133a-1, mir-133a-2, | 181a-2, mir-181b, mir-181b-1, |
| | mir-133b, mir-135a, mir-143, mir- | mir-181b-2, mir-181c, mir- |
| | 145, mir-146a, mir-154, mir-15a, mir- | 181d, mir-182, mir-182-5p, |
| | 187, mir-188-5p, mir-199b, mir-200b, | mir-183, mir-18a, mir-204, |
| | mir-203, mir-205, mir-212, mir-218, | mir-20a, mir-21, mir-221, mir- |
| | mir-221, mir-224, mir-23a, mir-23b, | 223-3p, mir-31, mir-429, mir- |
| | mir-25, mir-26a, mir-26b, mir-29b, | 96 |
| | mir-302a, mir-30a, mir-30b, mir-30c- | |
| | 1, mir-30c-2, mir-30d, mir-30e, mir- | |
| | 31, mir-330, mir-331-3p, mir-34a, | |
| | mir-34b, mir-34c, mir-374b, mir- | |
| | 449a, mir-4723-5p, mir-497, mir-628- | |
| | 5p, mir-642a-5p, mir-720, mir-940 | |
| acute lymphoblastic | let-7b, mir-124a, mir-142-3p | mir-128 |
| leukemia | | |
| malignant melanoma | let-7b, mir-101, mir-125b, mir-1280, | mir-126, mir-141, mir-15b, |
| | mir-143, mir-146a, mir-146b, mir- | mir-17, mir-17-5p, mir-182, |
| | 155, mir-17, mir-184, mir-185, mir- | mir-18a, mir-193b, mir-200a, |
| | 18b, mir-193b, mir-200c, mir-203, | mir-200b, mir-200c, mir-20a, |
| | mir-204, mir-205, mir-206, mir-20a, | mir-21, mir-210, mir-214, mir- |
| | mir-211, mir-218, mir-26a, mir-31, | 221, mir-222, mir-429, mir- |
| | mir-33a, mir-34a, mir-34c, mir-376a, | 455-5p, mir-532-5p, mir-638, |
| | mir-376c, mir-573, mir-7, mir-9, mir- | mir-92a |
| | 98 | |
| renal clear cell | let-7b, let-7c, mir-138, mir-141, mir- | mir-122, mir-155, mir-630 |
| carcinoma | 200c, mir-204, mir-218, mir-335, | |
| | mir-377, mir-506 | |
| acute myeloid | let-7c, mir-17, mir-181a, mir-20a, | mir-125b, mir-126-5p, mir- |
| leukemia | mir-223, mir-26a, mir-29a, mir-30c, | 128, mir-155, mir-29a, mir-32, |
| | mir-7 | mir-331, mir-370, mir-378 |
| acute promyelocytic | let-7c, mir-107, mir-342 | mir-181a, mir-181b, mir-92a |
| leukemia | | |
| head and neck | let-7d, mir-1, mir-107, mir-128, mir- | mir-106b, mir-134, mir-16, |
| squamous cell | 133a, mir-138, mir-149, mir-200c, | mir-184, mir-196a, mir-21, |
| carcinoma | mir-205, mir-218, mir-27a*, mir-29a, | mir-25, mir-30a-5p, mir-31, |
| | mir-29b-1, mir-29b-2, mir-29c, mir- | mir-372, mir-93 |
| | 300, mir-34a, mir-363, mir-375, mir- | |
| | 874 | |
| oral cancer | let-7d, mir-218, mir-34a, mir-375, | mir-10b, mir-196a-1, mir- |
| | mir-494 | 196a-2, mir-196b, mir-21 |
| papillary thyroid | mir-101, mir-130b, mir-138, mir- | let-7e, mir-146b, mir-146b-5p, |
| carcinoma | 146a, mir-16, mir-195, mir-199a-3p, | mir-151-5p, mir-155, mir- |
| | mir-204-5p, mir-219-5p, mir-26a, | 181a-1, mir-181a-2, mir-181b- |
| | mir-34b, mir-613 | 1, mir-181b-2, mir-181c, mir- |
| | | 181d, mir-182, mir-183, mir- |
| | | 199b-5p, mir-21, mir-221, |
| | | mir-222, mir-339-5p, mir-34a |
| glioblastoma | let-7g-5p, mir-100, mir-101, mir- | mir-10b, mir-125b, mir-127- |
| | 106a, mir-124, mir-124a, mir-125a, | 3p, mir-148a, mir-18a, mir- |
| | mir-125a-5p, mir-125b, mir-127-3p, | 196a, mir-196a-1, mir-196a-2, |
| | mir-128, mir-129, mir-136, mir-137, | mir-196b, mir-21, mir-210, |
| | mir-139-5p, mir-142-3p, mir-143, | mir-210-3p, mir-223, mir-340, |
| | mir-145, mir-146b-5p, mir-149, mir- | mir-576-5p, mir-626, mir-92b |
| | 152, mir-153, mir-195, mir-21, mir- | |
| | 212-3p, mir-219-5p, mir-222, mir- | |
| | 29b, mir-31, mir-3189-3p, mir-320, | |
| | mir-320a, mir-326, mir-330, mir-331- | |
| | 3p, mir-340, mir-342, mir-34a, mir- | |
| | 376a, mir-449a, mir-483-5p, mir-503, | |
| | mir-577, mir-663, mir-7, mir-744 | |
| ovarian cancer | let-7i, mir-100, mir-124, mir-125b, | mir-106a, mir-141, mir-148b, |
| | mir-129-5p, mir-130b, mir-133a, mir- | mir-181b, mir-182, mir-200a, |
| | 137, mir-138, mir-141, mir-145, mir- | mir-200c, mir-205, mir-20a, |
| | 148a, mir-152, mir-153, mir-155, | mir-21, mir-210, mir-214, mir- |
| | mir-199a, mir-200a, mir-200b, mir- | 221, mir-224-5p, mir-23b, |
| | 200c, mir-212, mir-335, mir-34a, mir- | mir-25, mir-26a, mir-27a, mir- |
| | 34b, mir-34c, mir-409-3p, mir-411, | 27b, mir-346, mir-378, mir- |
| | mir-429, mir-432, mir-449a, mir-494, | 424, mir-503, mir-572, mir-9, |
| | mir-497, mir-498, mir-519d, mir-655, | mir-96 |
| | mir-9, mir-98 | |
| bladder cancer | mir-1, mir-101, mir-1180, mir-1236, | mir-103a-3p, mir-10b, mir- |
| | mir-124-3p, mir-125b, mir-126, mir- | 135a, mir-137, mir-141, mir- |
| | 1280, mir-133a, mir-133b, mir-141, | 155, mir-17-5p, mir-182, mir- |
| | mir-143, mir-144, mir-145, mir-155, | 182-5p, mir-183, mir-185, |
| | mir-16, mir-18a, mir-192, mir-195, | mir-19a, mir-203, mir-205, |
| | mir-200a, mir-200b, mir-200c, mir- | mir-210, mir-221, mir-222, |
| | 203, mir-205, mir-214, mir-218, mir- | mir-223, mir-23a, mir-23b, |
| | 23b, mir-26a, mir-29c, mir-320c, mir- | mir-26b, mir-639, mir-96 |
| | 34a, mir-370, mir-409-3p, mir-429, | |
| | mir-451, mir-490-5p, mir-493, mir- | |
| | 576-3p, mir-99a | |
| chordoma | mir-1, mir-222, mir-31, mir-34a, mir- | mir-140-3p, mir-148a |
| | 608 | |
| kidney cancer | mir-1, mir-145, mir-1826, mir-199a, | mir-183, mir-21, mir-210, mir- |
| | mir-199a-3p, mir-203, mir-205, mir- | 223 |
| | 497, mir-508-3p, mir-509-3p | |
| cervical carcinoma | mir-100, mir-101, mir-15a, mir-16, | mir-133b, mir-21, mir-25, mir- |
| | mir-34a, mir-886-5p, mir-99a, mir- | 373 |
| | 99b | |
| mesenchymal cancer | mir-100, mir-141, mir-199b-5p, mir- | mir-125b-1-3p, mir-182 |
| | 200a, mir-200b, mir-200c, mir-29a, | |
| | mir-29b-1, mir-29b-1-5p, mir-29b-2, | |
| | mir-29c, mir-335, mir-429, mir-99a | |
| oral squamous cell | mir-100, mir-124, mir-1250, mir- | mir-125b, mir-126, mir-146a, |
| carcinoma | 125b, mir-126, mir-1271, mir-136, | mir-146b, mir-155, mir-181b, |
| | mir-138, mir-145, mir-147, mir-148a, | mir-196a-1, mir-196a-2, mir- |
| | mir-181a, mir-206, mir-220a, mir- | 196b, mir-21, mir-221, mir- |
| | 26a, mir-26b, mir-29a, mir-32, mir- | 222, mir-24, mir-27b, mir-31, |
| | 323-5p, mir-329, mir-338, mir-370, | mir-345 |
| | mir-410, mir-429, mir-433, mir-499a- | |
| | 5p, mir-503, mir-506, mir-632, mir- | |
| | 646, mir-668, mir-877, mir-9 | |
| ovarian carcinoma | mir-100, mir-101, mir-34b, mir-34c, | mir-148b, mir-182 |
| | mir-532-5p | |
| cholangiocarcinoma | mir-101, mir-144, mir-200b, mir- | mir-17, mir-18a, mir-19a, mir- |
| | 200c | 19b, mir-20a, mir-21, mir-26a, |
| | | mir-92a |
| endometrial cancer | mir-101, mir-130a, mir-130b, mir- | mir-106a, mir-145, mir-155, |
| | 134, mir-143, mir-145, mir-152, mir- | mir-182, mir-200b, mir-200c, |
| | 205, mir-223, mir-301a, mir-301b, | mir-205, mir-21, mir-222-3p, |
| | mir-30c, mir-34a, mir-34c, mir-424, | mir-25, mir-93 |
| | mir-449a, mir-543 | |
| esophageal cancer | mir-124, mir-126, mir-140, mir-197, | mir-101, mir-10b, mir-130a, |
| | mir-203, mir-218, mir-223, mir-30b, | mir-141, mir-143, mir-146b, |
| | mir-375, mir-454, mir-486, mir-574- | mir-15a, mir-183, mir-196b, |
| | 3p | mir-200a, mir-203, mir-205, |
| | | mir-21, mir-210, mir-221, mir- |
| | | 27a, mir-28-3p, mir-31, mir- |
| | | 452, mir-96, mir-99b |
| liver cancer | mir-101, mir-122, mir-132, mir-140- | mir-1301, mir-155, mir-21, |
| | 5p, mir-145, mir-148b, mir-31, mir- | mir-221, mir-27a, mir-525-3p |
| | 338-3p, mir-433 | |
| pancreatic cancer | mir-101, mir-1181, mir-124, mir- | mir-10a, mir-10b, mir-132, |
| | 1247, mir-133a, mir-141, mir-145, | mir-15a, mir-17-5p, mir-181a, |
| | mir-146a, mir-148a, mir-148b, mir- | mir-18a, mir-191, mir-196a, |
| | 150*, mir-150-5p, mir-152, mir-15a, | mir-21, mir-212, mir-214, mir- |
| | mir-198, mir-203, mir-214, mir-216a, | 222, mir-27a, mir-301a, mir- |
| | mir-29c, mir-335, mir-34a, mir-34b, | 301a-3p, mir-367, mir-424-5p, |
| | mir-34c, mir-373, mir-375, mir-410, | mir-7, mir-92, mir-99a |
| | mir-497, mir-615-5p, mir-630, mir-96 | |
| retinoblastoma | mir-101, mir-183, mir-204, mir-34a, | mir-181b, mir-21 |
| | mir-365b-3p, mir-486-3p, mir-532-5p | |
| cervical squamous | mir-106a, mir-124, mir-148a, mir- | mir-205 |
| cell carcinoma | 214, mir-218, mir-29a, mir-375 | |
| clear cell renal cell | mir-106a-5p, mir-135a-5p, mir-206 | mir-142-5p, mir-155, mir-21- |
| cancer | | 5p |
| laryngeal carcinoma | | mir-106b, mir-16, mir-21, mir- |
| | | 27a, mir-423-3p |
| medulloblastoma | mir-124, mir-128a, mir-199b-5p, mir- | mir-106b, mir-17, mir-18a, |
| | 206, mir-22, mir-31, mir-383 | mir-19a, mir-19b, mir-20a, |
| | | mir-30b, mir-30d, mir-92 |
| pituitary carcinoma | | mir-106b, mir-122, mir-20a, |
| | | mir-493 |
| prostate carcinoma | mir-107 | |
| cervical cancer | mir-143, mir-145, mir-17-5p, mir- | mir-10a, mir-155, mir-181a, |
| | 203, mir-214, mir-218, mir-335, mir- | mir-181b, mir-196a, mir-19a, |
| | 342-3p, mir-372, mir-424, mir-491- | mir-19b, mir-205, mir-20a, |
| | 5p, mir-497, mir-7, mir-99a, mir-99b | mir-21, mir-215, mir-224, mir- |
| | | 31, mir-494, mir-590-5p, mir- |
| | | 92a, mir-944 |
| chronic | mir-10a, mir-146a, mir-150, mir-151, | mir-424, mir-96 |
| myelogenous | mir-155, mir-2278, mir-26a, mir-30e, | |
| leukemia | mir-31, mir-326, mir-564 | |
| gastrointestinal | mir-122a, mir-148a, mir-152 | |
| cancer | | |
| anaplastic | mir-124, mir-137 | |
| astrocytoma | | |
| astrocytoma | mir-124-3p, mir-181b-5p, mir-200b, | mir-335 |
| | mir-3189-3p | |
| epithelial ovarian | mir-124a, mir-192, mir-193a, mir-7 | mir-372, mir-373 |
| cancer | | |
| mantle cell | mir-142-3p, mir-142-5p, mir-150, | mir-124a, mir-155, mir-17, |
| lymphoma | mir-223, mir-29a, mir-29b, mir-29c | mir-18a, mir-19a, mir-19b, |
| | | mir-20a, mir-92a |
| chronic lymphocytic | mir-125b, mir-138, mir-15a, mir-15b, | mir-150, mir-155 |
| leukemia | mir-16, mir-16-1, mir-16-1-3 p, mir- | |
| | 16-2, mir-181a, mir-181b, mir-195, | |
| | mir-223, mir-29b, mir-34b, mir-34c, | |
| | mir-424 | |
| follicular cancer | NA | mir-125b |
| malignant | mir-126 | |
| mesothelioma | | |
| small cell lung | mir-126, mir-138, mir-27a | mir-25 |
| cancer | | |
| meningioma | mir-128, mir-200a | mir-224, mir-335 |
| laryngeal squamous | mir-129-5p, mir-203, mir-205, mir- | mir-21, mir-9, mir-93 |
| cell carcinoma | 206, mir-24, mir-370, mir-375 | |
| medullary thyroid | mir-129-5p | mir-183 |
| carcinoma | | |
| lung | mir-1297, mir-141, mir-145, mir-16, | mir-150, mir-155, mir-31 |
| adenocarcinoma | mir-200a, mir-200b, mir-200c, mir- | |
| | 29b, mir-381, mir-409-3p, mir-429, | |
| | mir-451, mir-511, mir-99a | |
| pancreatic | mir-132, mir-375 | mir-301b |
| carcinoma | | |
| lung squamous cell | mir-133a, mir-218 | |
| carcinoma | | |
| multiple myeloma | mir-137, mir-197, mir-214 | mir-21 |
| squamous carcinoma | mir-15a, mir-16, mir-203, mir-205, | mir-137, mir-155, mir-184, |
| | mir-375 | mir-196a, mir-203, mir-21, |
| | | mir-221, mir-27a, mir-34a |
| uveal melanoma | mir-137, mir-144, mir-145, mir-182, | NA |
| | mir-34a, mir-34b, mir-34c, mir-9 | |
| anaplastic thyroid | mir-138 | mir-146b, mir-221, mir-222 |
| carcinoma | | |
| colorectal carcinoma | mir-139, mir-143, mir-145, mir-202- | mir-17, mir-182, mir-191, mir- |
| | 3p, mir-30a, mir-338-3p, mir-429, | 21, mir-95 |
| | mir-451, mir-93 | |
| malt lymphoma | | mir-142-5p, mir-155 |
| thyroid cancer | mir-144, mir-886-3p | |
| primary cns | mir-145, mir-193b, mir-199a, mir- | |
| lymphomas | 214 | |
| follicular thyroid | mir-199b | mir-146b, mir-183, mir-197, |
| carcinoma | | mir-221, mir-346 |
| gallbladder | mir-146b-5p | mir-155, mir-182 |
| carcinoma | | |
| adult t-cell leukemia | | mir-150 |
| anaplastic large-cell | | mir-155 |
| lymphoma | | |
| cutaneous t-cell | | mir-155 |
| lymphoma | | |
| diffuse large B-cell | | mir-155, mir-21 |
| lymphoma | | |
| rectal cancer | | mir-155, mir-200c, mir-21-5p, |
| | | mir-34a |
| tongue cancer | mir-15b, mir-200b | |
| b-cell lymphoma | mir-34a | mir-17, mir-18a, mir-19a, mir- |
| | | 19b, mir-20a, mir-92a |
| breast carcinoma | | mir-17, mir-18a, mir-19a, mir- |
| | | 19b, mir-20a, mir-24, mir-92a |
| nasopharyngeal | mir-218, mir-223, mir-29c | mir-17, mir-20a |
| cancer | | |
| gastric | mir-181b, mir-182, mir-200a, mir- | mir-23a, mir-27a, mir-373 |
| adenocarcinoma | 302b, mir-449a, mir-9 | |
| colorectal | | mir-182 |
| adenocarcinoma | | |
| colon carcinoma | mir-186, mir-30a-5p | mir-221, mir-23a |
| adrenal cortical | mir-195, mir-1974, mir-335, mir-497 | mir-21, mir-210, mir-483-3p, |
| carcinoma | | mir-483-5p |
| esophageal | mir-203 | mir-196a, mir-199a-3p, mir- |
| adenocarcinoma | | 199a-5p, mir-199b-3p, mir- |
| | | 200a, mir-223 |
| gastrointestinal | mir-218, mir-221, mir-222 | mir-196a |
| stromal tumor | | |
| uterine leiomyoma | mir-197 | |
| choriocarcinoma | mir-199b, mir-218, mir-34a | |
| follicular lymphoma | mir-202 | |
| basal cell carcinoma | mir-203 | |
| hypopharyngeal | | mir-203 |
| cancer | | |
| pancreatic | | mir-203, mir-301a |
| adenocarcinoma | | |
| rhabdomyosarcoma | mir-203 | |
| head and neck | NA | mir-21 |
| cancer | | |
| hypopharyngeal | mir-451a, mir-504 | mir-21 |
| squamous cell | | |
| carcinoma | | |
| t-cell lymphoma | mir-22 | |
| thyroid carcinoma | | mir-221, mir-222 |
| splenic marginal | mir-223 | |
| zone lymphoma | | |
| laryngeal cancer | | mir-23a |
| primary thyroid | mir-26a | |
| lymphoma | | |
| acute leukemia | mir-27a | |
| monocytic leukemia | mir-29a, mir-29b | |
| oral carcinoma | mir-375 | mir-31 |
| primary gallbladder | mir-335 | |
| carcinoma | | |
| endometrial serous | mir-34b | |
| adenocarcinoma | | |
| esophageal | mir-451 | |
| carcinoma | | |
| hepatoblastoma | | mir-492 |
| colonic | mir-627 | |
| adenocarcinoma | | |

TABLE 3
Exemplary tumor suppressive miRs

| Cancer | Down regulated tumor suppressive miR |
|---|---|
| acute leukemia | mir-27a |
| acute lymphoblastic leukemia | let-7b, mir-124a, mir-142-3p |
| acute myeloid leukemia | let-7c, mir-17, mir-181a, mir-20a, mir-223, mir-26a, mir- |
| | 29a, mir-30c, mir-720 |
| acute promyelocytic leukemia | let-7c, mir-107, mir-342 |
| adrenal cortical carcinoma | mir-195, mir-1974, mir-335, mir-497 |
| anaplastic astrocytoma | mir-124, mir-137 |
| anaplastic thyroid carcinoma | mir-138 |
| astrocytoma | mir-124-3p, mir-181b-5p, mir-200b, mir-3189-3p |
| basal cell carcinoma | mir-203 |
| b-cell lymphoma | mir-34a |
| bladder cancer | mir-1, mir-101, mir-1180, mir-1236, mir-124-3p, mir- |
| | 125b, mir-126, mir-1280, mir-133a, mir-133b, mir-141, |
| | mir-143, mir-144, mir-145, mir-155, mir-16, mir-18a, mir- |
| | 192, mir-195, mir-200a, mir-200b, mir-200c, mir-203, mir- |
| | 205, mir-214, mir-218, mir-23b, mir-26a, mir-29c, mir- |
| | 320c, mir-34a, mir-370, mir-409-3p, mir-429, mir-451, |
| | mir-490-5p, mir-493, mir-576-3p, mir-99a |
| breast cancer | let-7a, let-7a-1, let-7a-2, let-7a-3, let-7b, let-7c, let-7d, let- |
| | 7e, let-7f-1, let-7f-2, let-7g, let-7i, mir-100, mir-107, mir- |
| | 10a, mir-10b, mir-122, mir-124, mir-1258, mir-125a-5p, |
| | mir-125b, mir-126, mir-127, mir-129, mir-130a, mir-132, |
| | mir-133a, mir-143, mir-145, mir-146a, mir-146b, mir-147, |
| | mir-148a, mir-149, mir-152, mir-153, mir-15a, mir-16, |
| | mir-17-5p, mir-181a, mir-1826, mir-183, mir-185, mir- |
| | 191, mir-193a-3p, mir-193b, mir-195, mir-199b-5p, mir- |
| | 19a-3p, mir-200a, mir-200b, mir-200c, mir-205, mir-206, |
| | mir-211, mir-216b, mir-218, mir-22, mir-26a, mir-26b, |
| | mir-300, mir-30a, mir-31, mir-335, mir-339-5p, mir-33b, |
| | mir-34a, mir-34b, mir-34c, mir-374a, mir-379, mir-381, |
| | mir-383, mir-425, mir-429, mir-450b-3p, mir-494, mir- |
| | 495, mir-497, mir-502-5p, mir-517a, mir-574-3p, mir-638, |
| | mir-7, mir-720, mir-873, mir-874, mir-92a, mir-98, mir- |
| | 99a, mmu-mir-290-3p, mmu-mir-290-5p |
| bronchioloalveolar carcinoma | let-7a-1, let-7a-2, let-7a-3, let-7b, let-7c, let-7d, let-7e, let- |
| | 7f-1, let-7f-2, let-7g, let-7i, mir-98 |
| cervical cancer | mir-143, mir-145, mir-17-5p, mir-203, mir-214, mir-218, |
| | mir-335, mir-342-3p, mir-372, mir-424, mir-491-5p, mir- |
| | 497, mir-7, mir-99a, mir-99b |
| cervical carcinoma | mir-100, mir-101, mir-15a, mir-16, mir-34a, mir-886-5p, |
| | mir-99a, mir-99b |
| cervical squamous cell | mir-106a, mir-124, mir-148a, mir-214, mir-218, mir-29a, |
| carcinoma | mir-375 |
| cholangiocarcinoma | mir-101, mir-144, mir-200b, mir-200c |
| chondrosarcoma | let-7a, mir-100, mir-136, mir-145, mir-199a, mir-222, mir- |
| | 30a, mir-335, mir-376a |
| chordoma | mir-1, mir-222, mir-31, mir-34a, mir-608 |
| choriocarcinoma | mir-199b, mir-218, mir-34a |
| chronic lymphocytic leukemia | mir-125b, mir-138, mir-15a, mir-15b, mir-16, mir-16-1, |
| | mir-16-1-3p, mir-16-2, mir-181a, mir-181b, mir-195, mir- |
| | 223, mir-29b, mir-34b, mir-34c, mir-424 |
| chronic myelogenous leukemia | mir-10a, mir-138, mir-146a, mir-150, mir-151, mir-155, |
| | mir-16, mir-2278, mir-26a, mir-30e, mir-31, mir-326, mir- |
| | 564 |
| clear cell renal cell cancer | mir-106a-5p, mir-135a-5p, mir-206 |
| colon cancer | let-7a-1, let-7a-2, let-7a-3, let-7b, let-7c, let-7d, let-7e, let- |
| | 7f-1, let-7f-2, let-7g, let-7i, mir-100, mir-101, mir-126, |
| | mir-142-3p, mir-143, mir-145, mir-192, mir-200c, mir-21, |
| | mir-214, mir-215, mir-22, mir-25, mir-302a, mir-320, mir- |
| | 320a, mir-34a, mir-34c, mir-365, mir-373, mir-424, mir- |
| | 429, mir-455, mir-484, mir-502, mir-503, mir-93, mir-98 |
| colon carcinoma | mir-186, mir-30a-5p |
| colonic adenocarcinoma | mir-627 |
| colorectal cancer | let-7a, mir-1, mir-100, mir-101, mir-124, mir-125a, mir- |
| | 126, mir-129, mir-1295b-3p, mir-1307, mir-130b, mir-132, |
| | mir-133a, mir-133b, mir-137, mir-138, mir-139, mir-139- |
| | 5p, mir-140-5p, mir-143, mir-145, mir-148a, mir-148b, |
| | mir-149, mir-150-5p, mir-154, mir-15a, mir-15b, mir-16, |
| | mir-18a, mir-191, mir-192, mir-193a-5p, mir-194, mir-195, |
| | mir-196a, mir-198, mir-199a-5p, mir-200c, mir-203, mir- |
| | 204-5p, mir-206, mir-212, mir-215, mir-218, mir-22, mir- |
| | 224, mir-24-3p, mir-26b, mir-27a, mir-28-3p, mir-28-5p, |
| | mir-29b, mir-30a-3p, mir-30b, mir-320a, mir-328, mir- |
| | 338-3p, mir-342, mir-345, mir-34a, mir-34a-5p, mir-361- |
| | 5p, mir-375, mir-378, mir-378a-3p, mir-378a-5p, mir-409- |
| | 3p, mir-422a, mir-4487, mir-483, mir-497, mir-498, mir- |
| | 518a-3p, mir-551a, mir-574-5p, mir-625, mir-638, mir-7, |
| | mir-96-5p |
| colorectal carcinoma | mir-139, mir-143, mir-145, mir-202-3p, mir-30a, mir-338- |
| | 3p, mir-429, mir-451, mir-93 |
| endometrial cancer | mir-101, mir-130a, mir-130b, mir-134, mir-143, mir-145, |
| | mir-152, mir-205, mir-223, mir-301a, mir-301b, mir-30c, |
| | mir-34a, mir-34c, mir-424, mir-449a, mir-543 |
| endometrial serous | mir-34b |
| adenocarcinoma | |
| epithelial ovarian cancer | mir-124a, mir-192, mir-193a, mir-7 |
| esophageal adenocarcinoma | mir-203 |
| esophageal cancer | mir-124, mir-126, mir-140, mir-197, mir-203, mir-218, |
| | mir-223, mir-30b, mir-375, mir-454, mir-486, mir-574-3p |
| esophageal carcinoma | mir-451 |
| esophageal squamous cell | let-7a, let-7a-1, let-7a-2, let-7a-3, let-7b, let-7c, let-7d, let- |
| carcinoma | 7e, let-7f-1, let-7f-2, let-7g, let-7i, mir-1, mir-100, mir-101, |
| | mir-126, mir-1294, mir-133a, mir-133b, mir-138, mir-143, |
| | mir-145, mir-150, mir-185, mir-195, mir-200b, mir-203, |
| | mir-21, mir-210, mir-214, mir-218, mir-22, mir-27a, mir- |
| | 29b, mir-29c, mir-302b, mir-34a, mir-375, mir-494, mir- |
| | 518b, mir-655, mir-98, mir-99a |
| follicular lymphoma | mir-202 |
| follicular thyroid carcinoma | mir-199b |
| gallbladder carcinoma | mir-146b-5p |
| gastric adenocarcinoma | mir-181b, mir-182, mir-200a, mir-302b, mir-449a, mir-9 |
| gastric cancer | let-7a, let-7b, let-7g, mir-1, mir-101, mir-103a, mir-10a, |
| | mir-10b, mir-1207-5p, mir-122, mir-1228*, mir-124, mir- |
| | 124-3p, mir-125a-3p, mir-126, mir-1266, mir-127, mir- |
| | 1271, mir-129-1-3p, mir-129-2-3p, mir-129-3p, mir-129- |
| | 5p, mir-133a, mir-133b, mir-137, mir-141, mir-143, mir- |
| | 144, mir-145, mir-146a, mir-146a-5p, mir-148a, mir-148b, |
| | mir-149, mir-152, mir-155, mir-155-5p, mir-181a, mir- |
| | 181b, mir-182, mir-183, mir-185, mir-194, mir-195, mir- |
| | 197, mir-199a-3p, mir-200b, mir-200c, mir-202-3p, mir- |
| | 204, mir-204-5p, mir-205, mir-206, mir-210, mir-212, mir- |
| | 217, mir-218, mir-22, mir-23b, mir-24, mir-26a, mir-29a, |
| | mir-29a-3p, mir-29b, mir-29b-1, mir-29b-2, mir-29c, mir- |
| | 30a-5p, mir-30b, mir-31, mir-328, mir-329, mir-331-3p, |
| | mir-335-5p, mir-338, mir-338-3p, mir-34a, mir-34b, mir- |
| | 34c, mir-361-5p, mir-367, mir-375, mir-378, mir-409-3p, |
| | mir-410, mir-429, mir-433, mir-449, mir-449a, mir-490- |
| | 3p, mir-494, mir-497, mir-503, mir-506, mir-513b, mir- |
| | 520d-3p, mir-542-3p, mir-622, mir-625, mir-638, mir-663, |
| | mir-7, mir-874, mir-9 |
| gastrointestinal cancer | mir-122a, mir-148a, mir-152 |
| gastrointestinal stromal tumor | mir-218, mir-221, mir-222 |
| glioblastoma | let-7g-5p, mir-100, mir-101, mir-106a, mir-124, mir-124a, |
| | mir-125a, mir-125a-5p, mir-125b, mir-127-3p, mir-128, |
| | mir-129, mir-136, mir-137, mir-139-5p, mir-142-3p, mir- |
| | 143, mir-145, mir-146b-5p, mir-149, mir-152, mir-153, |
| | mir-195, mir-21, mir-212-3p, mir-219-5p, mir-222, mir- |
| | 29b, mir-31, mir-3189-3p, mir-320, mir-320a, mir-326, |
| | mir-330, mir-331-3p, mir-340, mir-342, mir-34a, mir-376a, |
| | mir-449a, mir-483-5p, mir-503, mir-577, mir-663, mir-7, |
| | mir-7-5p, mir-873 |
| glioma | let-7a, let-7f, mir-106a, mir-107, mir-122, mir-124, mir- |
| | 124-5p, mir-124a, mir-125b, mir-128, mir-136, mir-137, |
| | mir-139, mir-143, mir-145, mir-146a, mir-146b, mir-146b- |
| | 5p, mir-152, mir-15b, mir-16, mir-181a, mir-181a-1, mir- |
| | 181a-2, mir-181b, mir-181b-1, mir-181b-2, mir-181c, mir- |
| | 181d, mir-184, mir-185, mir-195, mir-199a-3p, mir-200a, |
| | mir-200b, mir-203, mir-204, mir-205, mir-218, mir-219- |
| | 5p, mir-23b, mir-26b, mir-27a, mir-29c, mir-320, mir-326, |
| | mir-328, mir-34a, mir-34c-3p, mir-34c-5p, mir-375, mir- |
| | 383, mir-451, mir-452, mir-483-5p, mir-495, mir-584, mir- |
| | 622, mir-656, mir-7, mir-98 |
| head and neck squamous cell | let-7d, mir-1, mir-107, mir-128, mir-13 3a, mir-138, mir- |
| carcinoma | 149, mir-200c, mir-205, mir-218, mir-27a*, mir-29a, mir- |
| | 29b-1, mir-29b-2, mir-29c, mir-300, mir-34a, mir-363, |
| | mir-375, mir-874 |
| hepatocellular carcinoma | let-7a-1, let-7a-2, let-7a-3, let-7b, let-7c, let-7d, let-7e, let- |
| | 7f, let-7f-1, let-7f-2, let-7g, let-7i, mir-1, mir-100, mir-101, |
| | mir-105, mir-122, mir-122a, mir-1236, mir-124, mir-125b, |
| | mir-126, mir-127, mir-1271, mir-128-3p, mir-129-5p, mir- |
| | 130a, mir-130b, mir-133a, mir-134, mir-137, mir-138, mir- |
| | 139, mir-139-5p, mir-140-5p, mir-141, mir-142-3p, mir- |
| | 143, mir-144, mir-145, mir-146a, mir-148a, mir-148b, mir- |
| | 150-5p, mir-15b, mir-16, mir-181a-5p, mir-185, mir-188- |
| | 5p, mir-193b, mir-195, mir-195-5p, mir-197, mir-198, mir- |
| | 199a, mir-199a-5p, mir-199b, mir-199b-5p, mir-200a, mir- |
| | 200b, mir-200c, mir-202, mir-203, mir-204-3p, mir-205, |
| | mir-206, mir-20a, mir-21, mir-21-3p, mir-211, mir-212, |
| | mir-214, mir-217, mir-218, mir-219-5p, mir-22, mir-223, |
| | mir-26a, mir-26b, mir-29a, mir-29b-1, mir-29b-2, mir-29c, |
| | mir-302b, mir-302c, mir-30a, mir-30a-3p, mir-335, mir- |
| | 338-3p, mir-33a, mir-34a, mir-34b, mir-365, mir-370, mir- |
| | 372, mir-375, mir-376a, mir-377, mir-422a, mir-424, mir- |
| | 424-5p, mir-433, mir-4458, mir-448, mir-450a, mir-451, |
| | mir-485-5p, mir-486-5p, mir-497, mir-503, mir-506, mir- |
| | 519d, mir-520a, mir-520b, mir-520c-3p, mir-582-5p, mir- |
| | 590-5p, mir-610, mir-612, mir-625, mir-637, mir-675, mir- |
| | 7, mir-877, mir-940, mir-941, mir-98, mir-99a |
| hypopharyngeal squamous cell | mir-451a, mir-504 |
| carcinoma | |
| kidney cancer | mir-1, mir-145, mir-1826, mir-199a, mir-199a-3p, mir-203, |
| | mir-205, mir-497, mir-508-3p, mir-509-3p |
| laryngeal squamous cell | mir-129-5p, mir-203, mir-205, mir-206, mir-24, mir-370, |
| carcinoma | mir-375 |
| liver cancer | mir-101, mir-122, mir-132, mir-140-5p, mir-145, mir- |
| | 148b, mir-31, mir-338-3p, mir-433 |
| lung adenocarcinoma | mir-1297, mir-141, mir-145, mir-16, mir-200a, mir-200b, |
| | mir-200c, mir-29b, mir-381, mir-409-3p, mir-429, mir- |
| | 451, mir-511, mir-99a |
| lung cancer | let-7a-1, let-7a-2, let-7a-3, let-7b, let-7c, let-7d, let-7e, let- |
| | 7f-1, let-7f-2, let-7g, let-7i, mir-1, mir-101, mir-133b, mir- |
| | 138, mir-142-5p, mir-144, mir-145, mir-1469, mir-146a, |
| | mir-153, mir-15a, mir-15b, mir-16-1, mir-16-2, mir-182, |
| | mir-192, mir-193a-3p, mir-194, mir-195, mir-198, mir- |
| | 203, mir-217, mir-218, mir-22, mir-223, mir-26a, mir-26b, |
| | mir-29c, mir-33a, mir-34a, mir-34b, mir-34c, mir-365, mir- |
| | 449a, mir-449b, mir-486-5p, mir-545, mir-610, mir-614, |
| | mir-630, mir-660, mir-7515, mir-9500, mir-98, mir-99b |
| lung squamous cell carcinoma | mir-133a, mir-218 |
| malignant melanoma | let-7b, mir-101, mir-125b, mir-1280, mir-143, mir-146a, |
| | mir-146b, mir-155, mir-17, mir-184, mir-185, mir-18b, |
| | mir-193b, mir-200c, mir-203, mir-204, mir-205, mir-206, |
| | mir-20a, mir-211, mir-218, mir-26a, mir-31, mir-33a, mir- |
| | 34a, mir-34c, mir-376a, mir-376c, mir-573, mir-7-5p, mir- |
| | 9, mir-98 |
| malignant mesothelioma | mir-126 |
| mantle cell lymphoma | mir-142-3p, mir-142-5p, mir-150, mir-223, mir-29a, mir- |
| | 29b, mir-29c |
| medullary thyroid carcinoma | mir-129-5p |
| medulloblastoma | mir-124, mir-128a, mir-199b-5p, mir-206, mir-22, mir-31, |
| | mir-383 |
| meningioma | mir-128, mir-200a |
| mesenchymal cancer | mir-100, mir-141, mir-199b-5p, mir-200a, mir-200b, mir- |
| | 200c, mir-29a, mir-29b-1, mir-29b-1-5p, mir-29b-2, mir- |
| | 29c, mir-335, mir-429, mir-99a |
| monocytic leukemia | mir-29a, mir-29b |
| multiple myeloma | mir-137, mir-197, mir-214 |
| nasopharyngeal cancer | mir-218, mir-223, mir-29c |
| nasopharyngeal carcinoma | let-7a, let-7a-1, let-7a-2, let-7a-3, let-7b, let-7c, let-7d, let- |
| | 7e, let-7f-1, let-7f-2, let-7g, let-7i, mir-1, mir-101, mir-124, |
| | mir-138, mir-143, mir-145, mir-148a, mir-200b, mir-204, |
| | mir-216b, mir-223, mir-29c, mir-320a, mir-324-3p, mir- |
| | 34c, mir-375, mir-378, mir-451, mir-506, mir-9, mir-98 |
| neuroblastoma | let-7a-1, let-7a-2, let-7a-3, let-7b, let-7c, let-7d, let-7e, let- |
| | 7f-1, let-7f-2, let-7g, let-7i, mir-124, mir-137, mir-145, |
| | mir-181c, mir-184, mir-200a, mir-29a, mir-335, mir-338- |
| | 3p, mir-34a, mir-449a, mir-885-5p, mir-98 |
| non-small cell lung cancer | let-7a, let-7c, mir-1, mir-100, mir-101, mir-106a, mir-107, |
| | mir-124, mir-125a-3p, mir-125a-5p, mir-126, mir-126*, |
| | mir-129, mir-133a, mir-137, mir-138, mir-140, mir-143, |
| | mir-145, mir-146a, mir-146b, mir-148a, mir-148b, mir- |
| | 149, mir-152, mir-153, mir-154, mir-155, mir-15a, mir-16, |
| | mir-17-5p, mir-181a-1, mir-181a-2, mir-181b, mir-181b-1, |
| | mir-181b-2, mir-181c, mir-181d, mir-184, mir-186, mir- |
| | 193b, mir-195, mir-199a, mir-204, mir-212, mir-221, mir- |
| | 224, mir-26b, mir-27a, mir-27b, mir-29a, mir-29b, mir- |
| | 29c, mir-30a, mir-30b, mir-30c, mir-30d, mir-30d-5p, mir- |
| | 30e-5p, mir-32, mir-335, mir-338-3p, mir-340, mir-342-3p, |
| | mir-34a, mir-34b, mir-361-3p, mir-365, mir-373, mir-375, |
| | mir-429, mir-449a, mir-4500, mir-451, mir-4782-3p, mir- |
| | 497, mir-503, mir-512-3p, mir-520a-3p, mir-526b, mir- |
| | 625*, mir-96, mir-99a |
| oral cancer | let-7d, mir-218, mir-34a, mir-375, mir-494 |
| oral carcinoma | mir-375 |
| oral squamous cell carcinoma | mir-100, mir-124, mir-1250, mir-125b, mir-126, mir-1271, |
| | mir-136, mir-138, mir-145, mir-147, mir-148a, mir-181a, |
| | mir-206, mir-220a, mir-26a, mir-26b, mir-29a, mir-32, |
| | mir-323-5p, mir-329, mir-338, mir-370, mir-410, mir-429, |
| | mir-433, mir-499a-5p, mir-503, mir-506, mir-632, mir- |
| | 646, mir-668, mir-877, mir-9 |
| osteosarcoma | let-7a, mir-1, mir-100, mir-101, mir-122, mir-124, mir- |
| | 125b, mir-126, mir-127-3p, mir-132, mir-133a, mir-141, |
| | mir-142-3p, mir-142-5p, mir-143, mir-144, mir-145, mir- |
| | 153, mir-16, mir-183, mir-194, mir-195, mir-199a-3p, mir- |
| | 204, mir-212, mir-217, mir-218, mir-22, mir-23a, mir-24, |
| | mir-26a, mir-26b, mir-29b, mir-32, mir-320, mir-335, mir- |
| | 33b, mir-340, mir-34a, mir-34b, mir-34c, mir-375, mir- |
| | 376c, mir-382, mir-3928, mir-424, mir-429, mir-449a, mir- |
| | 451, mir-454, mir-503, mir-519d, mir-646 |
| ovarian cancer | let-7i, mir-100, mir-124, mir-125b, mir-129-5p, mir-130b, |
| | mir-133a, mir-137, mir-138, mir-141, mir-145, mir-148a, |
| | mir-152, mir-153, mir-155, mir-199a, mir-200a, mir-200b, |
| | mir-200c, mir-212, mir-335, mir-34a, mir-34b, mir-34c, |
| | mir-409-3p, mir-411, mir-429, mir-432, mir-449a, mir- |
| | 494, mir-497, mir-498, mir-519d, mir-655, mir-9, mir-98 |
| ovarian carcinoma | mir-100, mir-101, mir-34b, mir-34c, mir-532-5p |
| pancreatic cancer | mir-101, mir-1181, mir-124, mir-1247, mir-133a, mir-141, |
| | mir-145, mir-146a, mir-148a, mir-148b, mir-150*, mir- |
| | 150-5p, mir-152, mir-15a, mir-198, mir-203, mir-214, mir- |
| | 216a, mir-29c, mir-335, mir-34a, mir-34b, mir-34c, mir- |
| | 373, mir-375, mir-410, mir-497, mir-615-5p, mir-630, mir- |
| | 96 |
| pancreatic carcinoma | mir-132, mir-375 |
| pancreatic ductal | let-7a, let-7a-1, let-7a-2, let-7a-3, let-7b, let-7c, let-7d, let- |
| adenocarcinoma | 7e, let-7f-1, let-7f-2, let-7g, let-7i, mir-126, mir-135a, mir- |
| | 143, mir-144, mir-145, mir-148a, mir-150, mir-15a, mir- |
| | 16, mir-200a, mir-200b, mir-200c, mir-217, mir-218, mir- |
| | 337, mir-375, mir-494, mir-615-5p, mir-98 |
| papillary thyroid carcinoma | mir-101, mir-130b, mir-138, mir-146a, mir-16, mir-195, |
| | mir-199a-3p, mir-204-5p, mir-219-5p, mir-26a, mir-34b, |
| | mir-613 |
| primary cns lymphomas | mir-145, mir-193b, mir-199a, mir-214 |
| primary gallbladder carcinoma | mir-335 |
| primary thyroid lymphoma | mir-26a |
| prostate cancer | let-7a-3p, let-7c, mir-100, mir-101, mir-105, mir-124, mir- |
| | 128, mir-1296, mir-130b, mir-133a-1, mir-133a-2, mir- |
| | 133b, mir-135a, mir-143, mir-145, mir-146a, mir-154, mir- |
| | 15a, mir-187, mir-188-5p, mir-199b, mir-200b, mir-203, |
| | mir-205, mir-212, mir-218, mir-221, mir-224, mir-23a, |
| | mir-23b, mir-25, mir-26a, mir-26b, mir-29b, mir-302a, |
| | mir-30a, mir-30b, mir-30c-1, mir-30c-2, mir-30d, mir-30e, |
| | mir-31, mir-330, mir-331-3p, mir-34a, mir-34b, mir-34c, |
| | mir-374b, mir-449a, mir-4723-5p, mir-497, mir-628-5p, |
| | mir-642a-5p, mir-765, mir-940 |
| prostate carcinoma | mir-107 |
| renal cell carcinoma | let-7a, let-7d, mir-1, mir-106a*, mir-126, mir-1285, mir- |
| | 129-3p, mir-1291, mir-133a, mir-135a, mir-138, mir-141, |
| | mir-143, mir-145, mir-182-5p, mir-199a-3p, mir-200a, |
| | mir-205, mir-218, mir-28-5p, mir-30a, mir-30c, mir-30d, |
| | mir-34a, mir-378, mir-429, mir-509-3p, mir-509-5p, mir- |
| | 646 |
| renal clear cell carcinoma | let-7b, let-7c, mir-138, mir-141, mir-200c, mir-204, mir- |
| | 218, mir-335, mir-377, mir-506 |
| retinoblastoma | mir-101, mir-183, mir-204, mir-34a, mir-365b-3p, mir- |
| | 486-3p, mir-532-5p |
| rhabdomyosarcoma | mir-203 |
| small cell lung cancer | mir-126, mir-138, mir-27a |
| splenic marginal zone lymphoma | mir-223 |
| squamous carcinoma | mir-15a, mir-16, mir-203, mir-205, mir-375 |
| t-cell lymphoma | mir-22 |
| thyroid cancer | mir-144, mir-886-3p |
| tongue cancer | mir-15b, mir-200b |
| uterine leiomyoma | mir-197 |
| uveal melanoma | mir-137, mir-144, mir-145, mir-182, mir-34a, mir-34b, |
| | mir-34c, mir-9 |

### REFERENCES

1. Loffler A et al., Blood. 2000; 95:2098-2103
2. Bargou R et al., Science. 2008; 321:974-977
3. Klinger M et al., Blood. 2012; 119:6226-6233
4. Topp M S et al., J Clin Oncol. 2011; 29:2493-2498.
5. Loffler A et al., Leukemia. 2003; 17:900-909
6. Topp M S et al., Blood. 2012; 120:5185-5187
7. Brischwein K et al., Mol Immunol. 2006; 43:1129-1143
8. Herrmann I etal., PLoS ONE. 2010; 5:e13474.
9. Cioffi M et al., Clin Cancer Res. 2012; 18:465-474
10. Witthauer J et al., 2009; 117:471-481
11. Osada T et al., Br J Cancer. 2010; 102:124-133
12. Lutterbuese R et al., J Immunother. 2009; 32:341-352.
13. Friedrich M et al., Molecular Cancer Therapeutics. 2012; 11:2664-2673
14. Laszlo G S et al., Blood. 2014; 123:554-561
15. Friedrich M et al., Mol Cancer Ther. 2014; 13:1549-1557
16. Lutterbuese R et al., PNAS, 2010; 107:12605-12610
17. Wuellner U et al., Antibodies 2015, 4, 426-440
18. Lopez-Albaitero A, Xu H, Guo H, et al. Oncoimmunology, 2017; 6(3):e1267891
19. Hammond S A et al., Cancer Research. 2007; 67:3927-3935
20. Torisu-Itakura H et al., J Immunother. 2011; 34:597-605
21. Yamamoto K et al., Biochem J. 2012; 445:135-144
22. Feldmann A et al., The Journal of Immunology. 2012; 189:3249-3259
23. Mack M et al., PNAS. 1995; 92:7021-7025
24. Zhang T et al., Cancer Research. 2011; 71:2066-2076
25. Godbersen C et al., Mol Cancer Ther. 2017 July; 16(7):1335-1346
26. Ross S L et al., PLoS One, 2017; e0183390

What is claimed is:

1. A lipid nanoparticle (LNP) comprising a recombinant DNA molecule comprising a polynucleotide sequence encoding a replication-competent viral genome, wherein the polynucleotide sequence is operably linked to a promoter sequence capable of binding a mammalian RNA polymerase II (Pol II) and is flanked by a 3′ ribozyme-encoding sequence and a 5′ ribozyme-encoding sequence,
wherein the polynucleotide encoding the replication-competent viral genome is non-viral in origin.

2. The LNP of claim 1, wherein the replication-competent viral genome is a single-stranded RNA (ssRNA) virus.

3. The LNP of claim 1, wherein the replication-competent viral genome is a single-stranded RNA (ssRNA) virus is a positive sense ((+)-sense) or a negative-sense ((−)-sense) ssRNA virus.

4. The LNP of claim 3, wherein the replication-competent viral genome is a (+)-sense ssRNA virus and the (+)-sense ssRNA virus is a Picornavirus.

5. The LNP of claim 4, wherein the Picornavirus is a Seneca Valley Virus (SVV) or a Coxsackievirus.

6. The LNP of any one of claims 1-5, wherein contacting the LNP with a cell results in production of viral particles by the cell, and wherein the viral particles are infectious and lytic.

7. The LNP of any one of claims 1-6, wherein the recombinant DNA molecule further comprises a polynucleotide sequence encoding an exogenous payload protein.

8. The LNP of claim 7, wherein the exogenous payload protein is a fluorescent protein, an enzymatic protein, a cytokine, a chemokine, or an antigen-binding molecule capable of binding to a cell surface receptor.

9. The LNP of claim 8, wherein the cytokine is selected from Flt3 ligand and IL-18.

10. The LNP of claim 8, wherein the chemokine is selected from CXCL10 and CCL4.

11. The LNP of claim 8, wherein the antigen-binding molecule is capable of binding to and inhibiting an immune checkpoint receptor.

12. The LNP of claim 11, wherein the immune checkpoint receptor is PD1.

13. The LNP of any one of claims 1-12, wherein a micro RNA (miRNA) target sequence (miR-TS) cassette is inserted into the nucleic acid sequence encoding the replication-competent viral genome, wherein the miR-TS cassette comprises one or more miRNA target sequences, and wherein expression of one or more of the corresponding miRNAs in a cell inhibits replication of the replication-competent viral genome in the cell.

14. The LNP of claim 13, wherein the one or more miRNAs are selected from miR-124, miR-1, miR-143, miR-128, miR-219, miR-219a, miR-122, miR-204, miR-217, miR-137, and miR-126.

15. The LNP of claim 14, wherein the miR-TS cassette comprises one or more copies of a miR-124 target sequence, one or more copies of a miR-1 target sequence, and one or more copies of a miR-143 target sequence.

16. The LNP of claim 14, wherein the miR-TS cassette comprises one or more copies of a miR-128 target sequence, one or more copies of a miR-219a target sequence, and one or more copies of a miR-122 target sequence.

17. The LNP of claim 14, wherein the miR-TS cassette comprises one or more copies of a miR-128 target sequence, one or more copies of a miR-204 target sequence, and one or more copies of a miR-219 target sequence.

18. The LNP of claim 14, wherein the miR-TS cassette comprises one or more copies of a miR-217 target sequence, one or more copies of a miR-137 target sequence, and one or more copies of a miR-126 target sequence.

19. The LNP of any one of claims 1-18, wherein the recombinant DNA molecule is a plasmid comprising the polynucleotide sequence encoding a replication-competent viral genome.

20. The LNP of any one of claims 1-19, wherein the LNP comprises a cationic lipid, a cholesterol, and a neutral lipid.

21. The LNP of claim 20, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

22. The LNP of claim 20 or 21, further comprising a phospholipid-polymer conjugate, wherein the phospholipid-polymer conjugate is 1, 2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

23. The LNP of any one of claims 1-22, wherein hyaluronan is conjugated to the surface of the LNP.

24. A therapeutic composition comprising a plurality of lipid nanoparticles according to any one of claims 1-23, wherein the plurality of LNPs have an average size of about 150 nm to about 500 nm.

25. The therapeutic composition of claim 24, wherein the plurality of LNPs have an average size of about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

26. The therapeutic composition of claim 24 or 25, wherein the plurality of LNPs have an average zeta-potential of less than about −20 mV, less than about −30 mV, less than about 35 mV, or less than about −40 mV.

27. The therapeutic composition of claim 26, wherein the plurality of LNPs have an average zeta-potential of between about −50 mV to about −20 mV, about −40 mV to about −20 mV, or about −30 mV to about −20 mV.

28. The therapeutic composition of claim 26 or 27, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

29. The therapeutic composition of any one of claims 24-28, wherein administering the therapeutic composition to a subject delivers the recombinant DNA polynucleotide to a target cell of the subject, and wherein the recombinant DNA polynucleotide produces an infectious virus capable of lysing the target cell of the subject.

30. The therapeutic composition of claim 29, wherein the composition is delivered intravenously or intratumorally.

31. The therapeutic composition of claim 29, wherein the target cell is a cancerous cell.

32. A method of inhibiting the growth of a cancerous tumor in a subject in need thereof comprising administering a therapeutic composition according to any one of claims 24-31 to the subject in need thereof, wherein administration of the composition inhibits the growth of the tumor.

33. The method of claim 32, wherein the administration is intratumoral or intravenous.

34. The method of claim 32 or 33, wherein the cancer is a lung cancer or a liver cancer.

35. A recombinant DNA molecule comprising a polynucleotide sequence encoding a replication-competent viral genome, wherein the polynucleotide sequence is operably linked to promoter sequence capable of binding a mammalian RNA polymerase II (Pol II) and is flanked by a 3′ ribozyme-encoding sequence and a 5′ ribozyme-encoding sequence,
wherein the polynucleotide encoding the replication-competent viral genome is non-viral in origin.

36. The recombinant DNA molecule of claim 35, wherein the encoded virus is a single-stranded RNA (ssRNA) virus

37. The recombinant DNA molecule of claim 36, wherein the ssRNA virus is a positive sense ((+)-sense) or a negative-sense ((−)-sense) ssRNA virus.

38. The recombinant DNA molecule of claim 37, wherein the (+)-sense ssRNA virus is a Picornavirus.

39. The recombinant DNA molecule of claim 38, wherein the Picornavirus is a Seneca Valley Virus (SVV) or a Coxsackievirus.

40. The recombinant DNA molecule of any one of claims 35-39, wherein the recombinant DNA molecule is capable of producing an infectious, lytic virus when introduced into a cell by a non-viral delivery vehicle.

41. The recombinant DNA molecule of any one of claims 35-39, wherein the recombinant DNA molecule further comprises a polynucleotide sequence encoding an exogenous payload protein.

42. The recombinant DNA molecule of claim 41, wherein the exogenous payload protein is a fluorescent protein, an enzymatic protein, a cytokine, a chemokine, a ligand for a cell-surface receptor, or an antigen-binding molecule capable of binding to a cell surface receptor.

43. The recombinant DNA molecule of claim 42, wherein the cytokine is IL-18.

44. The recombinant DNA molecule of claim 42, wherein the ligand for a cell-surface receptor is Flt3 ligand

45. The recombinant DNA molecule of claim 42, wherein the chemokine is selected from CXCL10 and CCL4.

46. The recombinant DNA molecule of claim 42, wherein the antigen-binding molecule is capable of binding to and inhibiting an immune checkpoint receptor.

47. The recombinant DNA molecule of claim 46, wherein the immune checkpoint receptor is PD1.

48. The recombinant DNA molecule of any one of claims 35-47, wherein a micro RNA (miRNA) target sequence (miR-TS) cassette is inserted into the nucleic acid sequence encoding the replication-competent viral genome, wherein the miR-TS cassette comprises one or more miRNA target sequences, and wherein expression of one or more of the corresponding miRNAs in a cell inhibits replication of the encoded virus in the cell.

49. The recombinant DNA molecule of claim 48, wherein the one or more miRNAs are selected from miR-124, miR-1, miR-143, miR-128, miR-219, miR-219a, miR-122, miR-204, miR-217, miR-137, and miR-126.

50. The recombinant DNA molecule of claim 49, wherein the miR-TS cassette comprises one or more copies of a miR-124 target sequence, one or more copies of a miR-1 target sequence, and one or more copies of a miR-143 target sequence.

51. The recombinant DNA molecule of claim 49, wherein the miR-TS cassette comprises one or more copies of a miR-128 target sequence, one or more copies of a miR-219a target sequence, and one or more copies of a miR-122 target sequence.

52. The recombinant DNA molecule of claim 49, wherein the miR-TS cassette comprises one or more copies of a miR-128 target sequence, one or more copies of a miR-204 target sequence, and one or more copies of a miR-219 target sequence.

53. The recombinant DNA molecule of claim 49, wherein the miR-TS cassette comprises one or more copies of a miR-217 target sequence, one or more copies of a miR-137 target sequence, and one or more copies of a miR-126 target sequence.

54. The recombinant DNA molecule of any one of claims 35-53, wherein the recombinant DNA molecule is a plasmid or a NanoV comprising the polynucleotide sequence encoding a replication-competent viral genome.

55. A recombinant DNA molecule comprising a polynucleotide sequence encoding a replication-competent viral genome,
wherein the polynucleotide sequence encoding the replication-competent virus is non-viral in origin, and
wherein the recombinant DNA molecule is capable of producing a replication-competent virus when introduced into a cell by a non-viral delivery vehicle.

56. The recombinant DNA molecule of claim 55, wherein the replication-competent viral genome is a genome of a DNA virus or a genome of an RNA virus.

57. The recombinant DNA molecule of claim 56, wherein the DNA genome or RNA genome is a double-stranded or a single-stranded virus.

58. The recombinant DNA molecule of claim 57, wherein the single stranded genome is a positive sense ((+)-sense) or negative sense ((−)-sense) genome.

59. The recombinant DNA molecule of claim 55, wherein the cell is a mammalian cell.

60. The recombinant DNA molecule of claim 59, wherein the cell is a mammalian cell present in a mammalian subject.

61. The recombinant DNA molecule of claim 55, wherein the replication-competent virus is selected from the group consisting of adenovirus, coxsackie virus, polio virus, Seneca valley virus, equine herpes virus, herpes simplex virus type 1 (HSV-1), lassa virus, murine leukemia virus, influenza A virus, influenza B virus, Newcastle disease virus, measles virus, parvovirus, reovirus, sindbis virus, vaccinia virus, myxoma virus, vesicular stomatitis virus (VSV), a mamba virus.

62. The recombinant DNA molecule of any of claims 55-61, further comprising one or more micro RNA (miRNA) target sequence (miR-TS) cassettes inserted into the polynucleotide encoding the replication-competent viral genome, wherein the miR-TS cassette comprises one or more miRNA target sequences, and wherein expression of one or more of the corresponding miRNAs in a cell inhibits replication of the encoded virus in the cell.

63. The recombinant DNA molecule of claim 62, wherein the one or more miR-TS cassettes is incorporated into the 5′ untranslated region (UTR) or 3′ UTR of one or more essential viral genes.

64. The recombinant DNA molecule of claim 63, wherein the one or more essential viral genes is selected from the group consisting of UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL50, UL52, UL53, UL54, US1, US3, US4, USS, US6, US7, US8, US12, ICP0, ICP4, ICP22, ICP27, ICP47, PB, F, B5R, SERO-1, Cap, Rev, VP1-4, nucleoprotein (N), phosphoprotein (P), matrix protein (M), glycoprotein (G), polymerase (L), E1, E2, E3, E3, VP1, VP2, VP3, VP4, 2A, 2B, 2C, 3A, 3B, 3C, and 3D.

65. The recombinant DNA molecule of claim 62, wherein the one or more miR-TS cassettes is incorporated into the 5′ untranslated region (UTR) or 3′ UTR of one or more non-essential genes.

66. The recombinant DNA molecule of any of claims 55-65, wherein the polynucleotide is inserted into a nucleic acid vector selected from a replicon, a plasmid, a cosmid, a phagemid, a transposon, a bacterial artificial chromosome, a yeast artificial chromosome, or an end-closed linear duplexed oncolytic virus (Ov) DNA molecule.

67. The recombinant DNA molecule of claim 55, wherein the polynucleotide is a DNA polynucleotide and further comprises a first AAV-derived inverted terminal repeat (ITR) on the 5′ end of the nucleic acid sequence encoding the replication-competent viral genome and a second AAV-derived ITR on the 3′ end of the nucleic acid sequence encoding the replication-competent viral genome.

68. The recombinant DNA molecule of claim 55, wherein the polynucleotide is a DNA polynucleotide and further comprises a first ribozyme encoding sequence immediately 3′ to the nucleic acid sequence encoding the replication-competent viral genome and a second ribozyme encoding sequence immediately 5′ to the nucleic acid sequence encoding the replication-competent viral genome.

69. The recombinant DNA molecule of claim 68, wherein the first and second ribozyme encoding sequences encode a Hammerhead ribozyme or a hepatitis delta virus ribozyme.

70. The recombinant DNA molecule of claim 55, wherein the promoter sequence is capable of binding a eukaryotic RNA polymerase.

71. The recombinant DNA molecule of claim 55, wherein the promoter sequence is capable of binding a mammalian RNA polymerase.

72. The recombinant DNA molecule of claim 55, wherein the polynucleotide is a DNA polynucleotide and the mammalian polymerase drives the transcription of an infectious, replication-competent RNA virus.

73. The recombinant DNA molecule of claim 55, wherein the polynucleotide is a DNA polynucleotide and the mammalian polymerase drives the transcription of an infectious, replication-competent DNA virus.

74. The recombinant DNA molecule of claim 55, wherein the promoter sequence selectively drives transcription of the polynucleotide in a cancer cell.

75. The recombinant DNA molecule of any one of claims 55-76, wherein the promoter sequence is derived a gene selected from the group consisting of hTERT, HE4, CEA, OC, ARF, CgA, GRP78, CXCR4, HMGB2, INSM1, Mesothelin, OPN, RAD51, TETP, H19, uPAR, ERBB2, MUC1, Frz1, or IGF2-P4.

76. The recombinant DNA molecule of any of claims 55-75, further comprising a nucleic acid sequence encoding a payload molecule selected from the group consisting of a cytotoxic polypeptide, a cytokine, a chemokine, an antigen binding molecule, a ligand for a cell surface receptor, a soluble receptor, an enzyme, a scorpion polypeptide, a snake polypeptide, a spider polypeptide, a bee polypeptide, a frog polypeptide, and a therapeutic nucleic acid.

77. The recombinant DNA molecule of claim 76, wherein one or more miR-TS cassettes is incorporated into the 5′ untranslated region (UTR) or the 3′ UTR sequence of the nucleic acid sequence encoding the payload molecule.

78. The recombinant DNA molecule of claim 76, wherein the cytotoxic polypeptide is selected from p53, diphtheria toxin (DT), Pseudomonas Exotoxin A (PEA), Type I ribosome inactivating proteins (RIPs), Type II RIPs, or Shiga-like toxin 1 (Slt1).

79. The recombinant DNA molecule of claim 76, wherein the enzyme is selected from a metalloproteinase, a collagenase, an elastase, a hyaluronidase, a caspase, a gelatinase, or an enzyme that is part of a gene directed enzyme prodrug therapy (GDEPT) system selected from herpes simplex virus thymidine kinase, cytosine deaminase, nitroreductase, carboxypeptidase G2, purine nucleoside phosphorylase, or cytochrome P450.

80. The recombinant DNA molecule of claim 79, wherein the gelatinase is fibroblast activation protein (FAP).

81. The recombinant DNA molecule of claim 79, wherein the metalloproteinase is a matrix metalloproteinase (e.g., MMP9) or ADAM17.

82. The recombinant DNA molecule of claim 76, wherein the cytokine is selected from the group consisting of osteopontin, IL-13, TGFβ, IL-35, IL-18, IL-15, IL-2, IL-12, IFNα, IFNβ, IFNγ.

83. The recombinant DNA molecule of claim 76, wherein the chemokine is selected from CXCL10, CCL4, CCL5, CXCL9, and CCL21.

84. The recombinant DNA molecule of claim 76, wherein the ligand for a cell-surface receptor is an NKG2D ligand, a neuropilin ligand, Flt3 ligand, a CD47 ligand.

85. The recombinant DNA molecule of claim 76, wherein the antigen-binding molecule binds to a cell-surface antigen selected from the group consisting of PD-1, PDL-1, CTLA4, CCR4, OX40, CD200R, CD47, CSF1R, EphA2, CD19, EpCAM, CEA, PSMA, CD33, EGFR, CCR4, CD200, CD40, CD47, HER2, DLL3, 4-1BB, 17-1A, GD2 and any one or more of the tumor antigens listed in Table 7.

86. The recombinant DNA molecule of claim 76, wherein the scorpion polypeptide is selected from the group consisting of chlorotoxin, BmKn-2, neopladine 1, neopladine 2, and mauriporin.

87. The recombinant DNA molecule of claim 76, wherein the snake polypeptide is selected from the group consisting of contortrostatin, apoxin-I, bothropstoxin-I, BJcuL, OHAP-1, rhodostomin, drCT-I, CTX-III, B1L, and ACTX-6.

88. The recombinant DNA molecule of claim 76, wherein the spider polypeptide is selected from the group consisting of latarcin and hyaluronidase.

89. The recombinant DNA molecule of claim 76, wherein the bee polypeptide is selected from the group consisting of melittin and apamin.

90. The recombinant DNA molecule of claim 76, wherein the frog polypeptide is selected from the group consisting of PsT-1, PdT-1, and PdT-2.

91. The recombinant DNA molecule of any one of claims 76-84, wherein the payload protein acts on an immune cell.

92. The recombinant DNA molecule of claim 91, wherein the immune cell is selected from a group consisting of a T cell, a B cell, a natural killer (NK) cell, an NKT cell, a macrophage, and/or a dendritic cell.

93. The recombinant DNA molecule of claim 76, wherein the payload polypeptide is a bipartite polypeptide comprising a first domain capable of binding a human cell surface antigen and a second domain capable of binding a human tumor cell antigen.

94. The recombinant DNA molecule of claim 93, wherein one or both domains of the bipartite polypeptide are antigen-binding molecules selected from the group consisting of an antibody, a single chain variable fragment (scFv), an F(ab), an immunoglobulin heavy chain variable domain, a diabody, a flexibody, a DOCK-AND-LOCK™ antibody, and a monoclonal anti-idiotypic antibody (mAb2).

95. The recombinant DNA molecule of claim 94, wherein the bipartite polypeptide is a dual-variable domain antibody (DVD-Ig™), a bi-specific T cell engager (BiTE™), a DuoBody®, a dual affinity retargeting (DART) polypeptide, or a Tandab®.

96. The recombinant DNA molecule of claim 94, wherein the antibody is an IgG antibody with an engineered Fc domain.

97. The recombinant DNA molecule of claim 76, wherein the therapeutic nucleic acid is an antagomir, a short-hair pin RNA (snRNA), a ribozyme, or an aptamer.

98. The recombinant DNA molecule of any of claims 62-97, wherein the polynucleotide does not replicate in or minimally replicates in a cell expressing a miRNA that binds to the miRNA target sequences comprised in the miR-TS cassette.

99. The recombinant DNA molecule of claim 98, wherein the miRNA is selected from Table 3.

100. The recombinant DNA molecule of claim 98, wherein the one or more miRNAs are selected from miR-124, miR-1, miR-143, miR-128, miR-219, miR-219a, miR-122, miR-204, miR-217, miR-137, and miR-126.

101. The recombinant DNA molecule of claim 100, wherein the miR-TS cassette comprises one or more copies of a miR-124 target sequence, one or more copies of a miR-1 target sequence, and one or more copies of a miR-143 target sequence.

102. The recombinant DNA molecule of claim 100, wherein the miR-TS cassette comprises one or more copies of a miR-128 target sequence, one or more copies of a miR-219a target sequence, and one or more copies of a miR-122 target sequence.

103. The recombinant DNA molecule of claim 100, wherein the miR-TS cassette comprises one or more copies of a miR-128 target sequence, one or more copies of a miR-204 target sequence, and one or more copies of a miR-219 target sequence.

104. The recombinant DNA molecule of claim 100, wherein the miR-TS cassette comprises one or more copies of a miR-217 target sequence, one or more copies of a miR-137 target sequence, and one or more copies of a miR-126 target sequence.

105. The recombinant DNA molecule of any one of claims 55-104, wherein the recombinant DNA molecule is a plasmid comprising the self-replicating polynucleotide.

106. A recombinant DNA molecule comprising:
(i) a first single-stranded DNA (ssDNA) molecule comprising a sense sequence of a viral genome; and
(ii) a second ssDNA molecule comprising an anti-sense sequence of the viral genome,
wherein each of the first and second ssDNA molecules comprise a 3′ inverted terminal repeat and a 5′ inverted terminal repeat and wherein the 3′ end of the sense ssDNA molecule is covalently linked to the 5′ end of the anti-sense ssDNA molecule, and the 5′ end of the sense ssDNA molecule is covalently linked to the 3′ end of the anti-sense ssDNA molecule to form an end-closed linear duplexed oncolytic virus (Ov) DNA molecule.

107. The recombinant DNA molecule of claim 106, wherein the encoded virus is a negative-sense or a positive-sense single stranded (ss) RNA virus.

108. The recombinant DNA molecule of claim 107, wherein the positive-sense ssRNA virus is a polio virus (PV).

109. The recombinant DNA molecule of claim 107, wherein the negative-sense ssRNA virus is a vesicular stomatitis virus (VSV) genome.

110. The recombinant DNA molecule of claim 106, wherein each of the first and second ssDNA molecules further comprises a ribozyme-encoding sequence immediately 5′ to the viral genome sequence and a ribozyme-encoding sequence immediately 3′ to the viral genome sequence.

111. The recombinant DNA molecule of any one of claims 106-110, wherein the viral genome comprises one or more micro-RNA (miRNA) target sequences inserted into one or more essential viral genes.

112. The recombinant DNA molecule of claim 111, wherein the one or more miRNA target sequences are inserted into the 3′ untranslated region (UTR) and/or the 5′ UTR of the one or more essential viral genes.

113. The recombinant DNA molecule of claim 111 or 112, wherein the one or more miRNA target sequences are inserted into at least 2, at least 3, at least 4, or more essential viral genes.

114. The recombinant DNA molecule of any one of claims 111-113, wherein at least 2, at least 3, or at least 4 miRNA target sequences are inserted into one or more essential viral genes.

115. The recombinant DNA molecule of claim 114, wherein the at least 2, at least 3, or at least 4 miRNA target sequences comprise target sequences for one miRNA.

116. The recombinant DNA molecule of claim 114, wherein the at least 2, at least 3, or at least 4 miRNA target sequences comprise target sequences for at least 2, at least 3, or at least 4 different miRNAs.

117. The recombinant DNA molecule of claim 106, wherein the viral genome is a VSV genome, and wherein the one or more miRNA target sequences are inserted into one or more of the genes encoding nucleoprotein (N), phosphoprotein (P), matrix protein (M), glycoprotein (G), and/or polymerase (L) proteins.

118. The recombinant DNA molecule of claim 106, wherein the viral genome is a PV genome, and wherein the one or more miRNA target sequences are inserted in one or more of the genes encoding the VP1, VP2, VP3, VP4, 2A, 2B, 2C, 3A, 3B (VPg), 3C, or 3D proteins.

119. The recombinant DNA molecule of any one of claims 106-118, wherein 3′ and 5′ ITRs are derived from AAV.

120. The recombinant DNA molecule of claim 119, wherein the AAV is AAV2.

121. A composition comprising an effective amount of the recombinant DNA molecule of any one of claims 1-120, and a carrier suitable for administration to a mammalian subject.

122. A particle comprising the recombinant DNA molecule of any one of claims 55-120.

123. The particle of claim 122, wherein the particle is biodegradable.

124. The particle of claim 123, wherein the particle is selected from the group consisting of a nanoparticle, an exosome, a liposome, and a lipoplex.

125. The particle of claim 124, wherein the exosome is a modified exosome derived from an intact exosome or an empty exosome.

126. The particle of claim 124, wherein the nanoparticle is a lipid nanoparticle (LNP) comprising a cationic lipid, a cholesterol, and a neutral lipid.

127. The LNP of claim 126, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

128. The LNP of claim 126 or 127, further comprising a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

129. The LNP of any one of claims 126-128, wherein hyaluronan is conjugated to the surface of the LNP.

130. A therapeutic composition comprising a plurality of lipid nanoparticles according to any one of claims 126-129, wherein the plurality of LNPs have an average size of about 150 nm to about 500 nm.

131. The therapeutic composition of claim 130, wherein the plurality of LNPs have an average size of about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

132. The therapeutic composition of claim 130 or 131, wherein the plurality of LNPs have an average zeta-potential of less than about −20 mV, less than about −30 mV, less than about 35 mV, or less than about −40 mV.

133. The therapeutic composition of claim 132, wherein the plurality of LNPs have an average zeta-potential of between about −50 mV to about −20 mV, about −40 mV to about −20 mV, or about −30 mV to about −20 mV.

134. The therapeutic composition of claim 131 or 132, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

135. The therapeutic composition of any one of claims 130-134, wherein delivery of the composition to a subject delivers the encapsulated DNA expression cassette to a target cell, and wherein the encapsulated DNA expression cassette produces an infectious virus capable of lysing the target cell.

136. The therapeutic composition of claim 135, wherein the composition is delivered intravenously or intratumorally.

137. The therapeutic composition of claim 136, wherein the target cell is a cancerous cell.

138. An inorganic particle comprising the polynucleotide of any one of claims 1-120.

139. The particle of claim 138, wherein the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

140. A composition comprising the particle of claim 138 or 139, wherein the average diameter of the particles is less than about 500 nm, is between about 250 nm and about 500 nm, or is about 350 nm.

141. A method of killing a cancerous cell comprising exposing the cancerous cell to the particle or composition of any one of claims 122-140, or a composition thereof, under conditions sufficient for the intracellular delivery of the particle to said cancerous cell, wherein the replication-competent virus produced by the encapsulated polynucleotide results in killing of the cancerous cell.

142. The method of claim 141, wherein the replication-competent virus is not produced in non-cancerous cells.

143. The method of claim 141 or 142, wherein the method is performed in vivo, in vitro, or ex vivo.

144. A method of treating a cancer in a subject comprising administering to a subject suffering from the cancer an effective amount of the particle or composition of any one of claims 122-140, or a composition thereof.

145. The method of claim 144, wherein the particle or composition thereof is administered intravenously, intranasally, as an inhalant, or is injected directly into a tumor.

146. The method of claim 144 or 145, wherein the particle or composition thereof is administered to the subject repeatedly.

147. The method of any of claims 144-146, wherein the subject is a mouse, a rat, a rabbit, a cat, a dog, a horse, a non-human primate, or a human.

148. The method of any of claims 144-147, wherein the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), and marginal zone lymphoma (MZL).

149. The method of claim 148, wherein the lung cancer is small cell lung cancer or non-small cell lung cancer.

150. The method of claim 148, wherein the liver cancer is hepatocellular carcinoma (HCC).

151. A method of producing a recombinant DNA molecule of any of the preceding claims comprising:
a. inserting the recombinant DNA molecule into a first viral expression vector, wherein the recombinant DNA molecule comprises a 5′ adeno-associated virus (AAV)-derived inverted terminal repeat (ITR) and a 3′ AAV-derived ITR end of the polynucleotide;
b. inserting polynucleotides encoding AAV proteins required for ITR-mediated replication into a second viral expression vector; and
c. intracellularly delivering the first and the second viral expression vectors to a cell,
wherein the recombinant DNA molecule is stably integrated into the genome, wherein the cell produces the ITR-flanked polynucleotides in amounts greater than would be produced in the absence of ITRs.

152. The method of claim 144, wherein the viral expression vector is a herpes virus or a baculovirus.


SEQUENCE LISTING (2 sequences)

SEQ ID NO: 1 - DNA, 8385 bp, Artificial Sequence
  Made in Lab - SVV Expression cassette
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 2 - DNA, 8523 bp, Artificial Sequence
  Made in Lab - CVA21 Expression cassette
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgtttaaacttaagcttgtttaactgatgagtccgtgaggacgaaacgagtaagctcgtcttaaaacagcctgtgggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggagaaaaaaaaaaaaaaaaaaaaaaaaaaaaaaggccggcatggtcccagcctcctcgctggcgccggctgggcaacatgcttcggcatggcgaatgggacgctcgagtctagagggcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg